(19) United States
(12) Patent Application Publication
(10) Pub. No.: US 2007/0185100 A1
(43) Pub. Date: Aug. 9, 2007

(54) Poly-heterocyclic compounds and their use as metabotropic glutamate receptor antagonists

(75) Inventors: Jalaj Arora, Cambridge (CA); Louise Edwards, Toronto (CA); Methvin Isaac, Toronto (CA); Annika Kers, Sodertalje (SE); Karin Staaf, Sodertalje (SE); Abdelmalik Slassi, Toronto (CA); Tomislav Stefanac, Toronto (CA); David Wensbo, Sodertalje (SE); Tao Xin, Woodbridge (CA); Bjorn Holm, Molndal (SE)

Correspondence Address: BIRCH, STEWART, KOLASCH & BIRCH, LLP, P.O. BOX 747, 8110 GATEHOUSE ROAD, SUITE 500 EAST, FALLS CHURCH, VA, 22040-0747, US

(73) Assignee: AstraZeneca AB, Sodertalje (SE)

(21) Appl. No.: 10/588,754

(22) PCT Filed: Feb. 17, 2005
(86) PCT No.: PCT/US05/05216

§ 371 Date: Feb. 2, 2007

Related U.S. Application Data
(60) Provisional application No. 60/545,292, filed on Feb. 18, 2004.

(51) Int. Cl. A61K 31/535

(52) U.S. Cl. 514/228.8; 514/252.1; 514/326; 544/139; 544/336; 546/192

(57) ABSTRACT

The present invention relates to new compounds of formula (I), wherein P, Q, X¹, X², X³, X⁴, X⁵, X⁶, R¹, R², R³, m, n, and p are as defined as in formula (I), or salts, or hydrates thereof, processes for their preparation and new intermediates used in the preparation thereof, pharmaceutical compositions containing said compounds and to the use of said compounds in therapy.

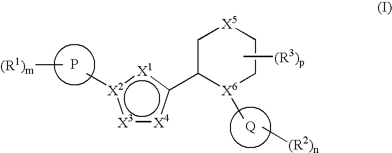

### FIELD OF THE INVENTION

The present invention relates to a new class of compounds, to pharmaceutical compositions containing said compounds and to the use of said compounds in therapy. The present invention further relates to processes for the preparation of said compounds and to new intermediates used in the preparation thereof.

### BACKGROUND OF THE INVENTION

Glutamate is the major excitatory neurotransmitter in the mammalian central nervous system (CNS). Glutamate produces its effects on central neurons by binding to and thereby activating cell surface receptors. These receptors have been divided into two major classes, the ionotropic and metabotropic glutamate receptors, based on the structural features of the receptor proteins, the means by which the receptors transduce signals into the cell, and pharmacological profiles.

The metabotropic glutamate receptors (mGluRs) are G protein-coupled receptors that activate a variety of intracellular second messenger systems following the binding of glutamate. Activation of mGluRs in intact mammalian neurons elicits one or more of the following responses: activation of phospholipase C; increases in phosphoinositide (PI) hydrolysis; intracellular calcium release; activation of phospholipase D; activation or inhibition of adenyl cyclase; increases or decreases in the formation of cyclic adenosine monophosphate (cAMP); activation of guanylyl cyclase; increases in the formation of cyclic guanosine monophosphate (cGMP); activation of phospholipase A₂; increases in arachidonic acid release; and increases or decreases in the activity of voltage- and ligand-gated ion channels. Schoepp et al., Trends Pharmacol. Sci. 14:13 (1993), Schoepp, Neurochem. Int. 24:439 (1994), Pin et al., Neuropharmacology 34:1 (1995), Bordi and Ugolini, Prog. Neurobiol. 59:55 (1999).

Eight distinct mGluR subtypes, termed mGluR1 through mGluR8, have been identified by molecular cloning. Nakanishi, Neuron 13:1031 (1994), Pin et al., Neuropharmacology 34:1 (1995), Knopfel et al., J. Med. Chem. 38:1417 (1995). Further receptor diversity occurs via expression of alternatively spliced forms of certain mGluR subtypes. Pin et al., PNAS 89:10331 (1992), Minakami et al., BBRC 199:1136 (1994), Joly et al., J. Neurosci. 15:3970 (1995).

Metabotropic glutamate receptor subtypes may be subdivided into three groups, Group I, Group II, and Group III mGluRs, based on amino acid sequence homology, the second messenger systems utilized by the receptors, and by their pharmacological characteristics. Group I mGluR comprises mGluR1, mGluR5 and their alternatively spliced variants. The binding of agonists to these receptors results in the activation of phospholipase C and the subsequent mobilization of intracellular calcium.

Neurological, Psychiatric and Pain Disorders.

Attempts at elucidating the physiological roles of Group I mGluRs suggest that activation of these receptors elicits neuronal excitation. Various studies have demonstrated that Group I mGluRs agonists can produce postsynaptic excitation upon application to neurons in the hippocampus, cerebral cortex, cerebellum, and thalamus, as well as other CNS regions. Evidence indicates that this excitation is due to direct activation of postsynaptic mGluRs, but it also has been suggested that activation of presynaptic mGluRs occurs, resulting in increased neurotransmitter release. Baskys, Trends Pharmacol. Sci. 15:92 (1992), Schoepp, Neurochem. Int. 24:439 (1994), Pin et al., Neuropharmacology 34:1 (1995), Watkins et al., Trends Pharmacol. Sci. 15:33 (1994).

Metabotropic glutamate receptors have been implicated in a number of normal processes in the mammalian CNS. Activation of mGluRs has been shown to be required for induction of hippocampal long-term potentiation and cerebellar long-term depression. Bashir et al., Nature 363:347 (1993), Bortolotto et al., Nature 368:740 (1994), Aiba et al., Cell 79:365 (1994), Aiba et al., Cell 79:377 (1994). A role for mGluR activation in nociception and analgesia also has been demonstrated. Meller et al., Neuroreport 4: 879 (1993), Bordi and Ugolini, Brain Res. 871:223 (1999). In addition, mGluR activation has been suggested to play a modulatory role in a variety of other normal processes including synaptic transmission, neuronal development, apoptotic neuronal death, synaptic plasticity, spatial learning, olfactory memory, central control of cardiac activity, waking, motor control and control of the vestibulo-ocular reflex. Nakanishi, Neuron 13: 1031 (1994), Pin et al., Neuropharmacology 34:1, Knopfel et al., J. Med. Chem. 38:1417 (1995). Further, Group I metabotropic glutamate receptors have been suggested to play roles in a variety of acute and chronic pathophysiological processes and disorders affecting the CNS. These include stroke, head trauma, anoxic and ischemic injuries, hypoglycemia, epilepsy, neurodegenerative disorders such as Alzheimer's disease, psychiatric disorders and pain. Schoepp et al., Trends Pharmacol. Sci. 14:13 (1993), Cunningham et al., Life Sci. 54:135 (1994), Hollman et al., Ann. Rev. Neurosci. 17:31 (1994), Pin et al., Neuropharmacology 34:1 (1995), Knopfel et al., J. Med. Chem. 38:1417 (1995), Spooren et al., Trends Pharmacol. Sci. 22:331 (2001), Gasparini et al. Curr. Opin. Pharmacol. 2:43 (2002), Neugebauer Pain 98:1 (2002). Much of the pathology in these conditions is thought to be due to excessive glutamate-induced excitation of CNS neurons. Because Group I mGluRs appear to increase glutamate-mediated neuronal excitation via postsynaptic mechanisms and enhanced presynaptic glutamate release, their activation probably contributes to the pathology. Accordingly, selective antagonists of Group I mGluR receptors could be therapeutically beneficial in all conditions underlain by excessive glutamate-induced excitation of CNS neurons, specifically as neuroprotective agents, analgesics or anticonvulsants.

Recent advances in the elucidation of the neurophysiological roles of metabotropic glutamate receptors generally and Group I in particular, have established these receptors as promising drug targets in the therapy of acute and chronic neurological and psychiatric disorders and chronic and acute pain disorders.

Gastro Intestinal Disorders

The lower esophageal sphincter (LES) is prone to relaxing intermittently. As a consequence, fluid from the stomach can pass into the esophagus since the mechanical barrier is temporarily lost at such times, an event hereinafter referred to as “G.I. reflux”. Gastro-esophageal reflux disease (GERD) is the most prevalent upper gastrointestinal tract disease. Current pharmacotherapy aims at reducing gastric acid secretion, or at neutralizing acid in the esophagus. The major mechanism behind G.I. reflux has been considered to depend on a hypotonic lower esophageal sphincter. However, e.g. Holloway & Dent (1990) Gastroenterol. Clin. N. Amer. 19, pp. 517-535, has shown that most reflux episodes occur during transient lower esophageal sphincter relaxations (TLESRs), i.e. relaxations not triggered by swallows. It has also been shown that gastric acid secretion usually is normal in patients with GERD.

The novel compounds according to the present invention are assumed to be useful for the inhibition of transient lower esophageal sphincter relaxations (TLESRs) and thus for treatment of gastro-esophageal reflux disorder (GERD).

The wording “TLESR”, transient lower esophageal sphincter relaxations, is herein defined in accordance with Mittal, R. K, Holloway, R. H., Penagini, R., Blackshaw, L. A., Dent, J., 1995; Transient lower esophageal sphincter relaxation. Gastroenterology 109, pp. 601-610.

The wording “G.I. reflux” is herein defined as fluid from the stomach being able to pass into the esophagus, since the mechanical barrier is temporarily lost at such times.

The wording “GERD”, gastro-esophageal reflux disease, is herein defined in accordance with van Heerwarden, M. A., Smout A. J. P. M., 2000; Diagnosis of reflux disease. Baillière's Clin. Gastroenterol. 14, pp. 759-774.

Because of their physiological and pathophysiological significance, there is a need for new potent mGluR agonists and antagonists that display a high selectivity for mGluR subtypes, particularly the Group I receptor subtype.

### SUMMARY OF THE INVENTION

In one aspect of the invention there is provided a compound according to formula I

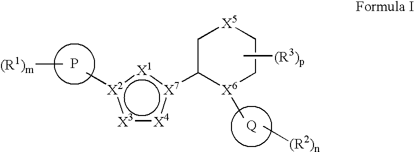

wherein

P is selected from aryl and heteroaryl;

R¹ is attached to P via a carbon atom on ring P and is selected from the group consisting of: hydroxy, halo, nitro, C₁₋₆alkylhalo, OC₁₋₆alkylhalo, C₁₋₆alkyl, OC₁₋₆alkyl, C₂₋₆alkenyl, OC₂₋₆alkenyl, C₂₋₆alkynyl, OC₂₋₆alkynyl, C₀₋₆alkylC₃₋₆cycloalkyl, OC₀₋₆alkylC₃₋₆cycloalkyl, C₀₋₆alkylaryl, OC₀₋₆alkylaryl, CHO, (CO)R⁵, O(CO)R⁵, O(CO)OR⁵, O(CNR⁵)OR⁵, C₁₋₆alkylOR⁵, OC₂₋₆alkylOR⁵, C₁₋₆alkyl(CO)R⁵, OC₁₋₆alkyl(CO)R⁵, C₀₋₆alkylCO₂R⁵, OC₁₋₆alkylCO₂R⁵, C₀₋₆alkylcyano, OC₂₋₆alkylcyano, C₀₋₆alkylNR⁵R⁶, OC₂₋₆alkylNR⁵R⁶, C₁₋₆alkyl(CO)NR⁵R⁶, OC₁₋₆alkyl(CO)NR⁵R⁶, C₀₋₆alkylNR⁵(CO)R⁶, OC₂₋₆alkylNR⁵(CO)R⁶, C₀₋₆alkylNR⁵(CO)NR⁵R⁶, C₀₋₆alkylSR⁵, OC₂₋₆alkylSR⁵, C₀₋₆alkyl(SO)R⁵, OC₂₋₆alkyl(SO)R⁵, C₀₋₆alkylSO₂R⁵, OC₂₋₆alkylSO₂R⁵, C₀₋₆alkyl(SO₂)NR⁵R⁶, OC₂₋₆alkyl(SO₂)NR⁵R⁶, C₀₋₆alkylNR⁵(SO₂)R⁶, OC₂₋₆alkylNR⁵(SO₂)R⁶, C₀₋₆alkylNR⁵(SO₂)NR⁵R⁶, OC₂₋₆alkylNR⁵(SO₂)NR⁵R⁶, (CO)NR⁵R⁶, O(CO)NR⁵R⁶, NR⁵OR⁶, C₀₋₆alkylNR⁵(CO)OR⁶, OC₂₋₆alkylNR⁵(CO)OR⁶, SO₃R⁵ and a 5- or 6-membered ring containing atoms independently selected from the group consisting of C, N, O and S;

X¹ is selected from the group consisting of: N, NR⁴ and CR⁴;

X² is selected from the group consisting of: C and N;

X³ is selected from the group consisting of: CR⁴, N and O;

X⁴ is selected from the group consisting of: CR⁴, N, NR⁴ and O;

X⁵ is selected from the group consisting of: a bond, CR⁴R^{4′}, NR⁴, O, S, SO and SO₂;

X⁶ is selected from the group consisting of: CR⁴ and N;

X⁷ is selected from the group consisting of: C and N;

R⁴ is independently selected from a group consisting of hydrogen, hydroxy, C₁₋₆alkyl, C₀₋₆alkylcyano, oxo, ═NR⁵, ═NOR⁵, C₁₋₄alkylhalo, halo, C₃₋₇cycloalkyl, O(CO)C₁₋₄alkyl, C₁₋₄alkyl(SO)C₀₋₄alkyl, C₁₋₄alkyl(SO₂)C₀₋₄alkyl, (SO)C₀₋₄alkyl, (SO₂)C₀₋₄alkyl, OC₁₋₄alkyl, C₁₋₄alkylOR⁵ and C₀₋₄alkylNR⁵R⁶;

Q is selected the group consisting of heterocycloalkyl and heteroaryl;

R² and R³ are independently selected from the group consisting of: hydroxy, C₀₋₆alkylcyano, oxo, ═NR⁵, ═NOR⁵, C₁₋₄alkylhalo, halo, C₁₋₆alkyl, C₃₋₆cycloalkyl, C₀₋₆alkylaryl, C₀₋₆alkylheteroaryl, C₁₋₆alkylcycloalkyl, C₀₋₆alkylheterocycloalkyl, OC₁₋₄alkyl, OC₀₋₆alkylaryl, O(CO)C₁₋₄alkyl, (CO)OC₁₋₄alkyl, C₀₋₄alkyl(S)C₀₋₄alkyl, C₁₋₄alkyl(SO)C₀₋₄alkyl, C₁₋₄alkyl(SO₂)C₀₋₄alkyl, (SO)C₀₋₄alkyl, (SO₂)C₀₋₄alkyl, C₁₋₄alkylOR⁵, C₀₋₄alkylNR⁵R⁶ and a 5- or 6-membered ring containing atoms independently selected from C, N, O and S, which ring may optionally be fused with a 5- or 6-membered ring containing atoms independently selected from the group consisting of C, N and O and wherein said ring and said fused ring may be substituted by one or more A;

wherein any C₁₋₆alkyl, aryl, or heteroaryl defined under R¹, R² and R³ may be substituted by one or more A;

A is selected from the group consisting of: hydrogen, hydroxy, halo, nitro, oxo, C₀₋₆alkylcyano, C₀₋₄alkylC₃₋₆cycloalkyl, C₁₋₆alkyl, —OC₁₋₆alkyl, C₁₋₆alkylhalo, OC₁₋₆alkylhalo, C₂₋₆alkenyl, C₀₋₃alkylaryl, C₀₋₆alkylOR⁵, OC₂₋₆alkylOR⁵, C₀₋₆alkylSR⁵, OC₂₋₆alkylSR⁵, (CO)R⁵, O(CO)R⁵, OC₂₋₆alkylcyano, OC₁₋₆alkylCO₂R⁵, O(CO)OR⁵, OC₁₋₆alkyl(CO)R⁵, C₁₋₆alkyl(CO)R⁵, NR⁵OR⁶, C₀₋₆NR⁵R⁶, OC₂₋₆alkylNR⁵R⁶, C₀₋₆alkyl(CO)NR⁵R⁶, OC₁₋₆alkyl(CO)NR⁵R⁶, OC₂₋₆alkylNR⁵(CO)R⁶, C₀₋₆alkylNR⁵(CO)R⁶, C₀₋₆alkylNR⁵(CO)NR⁵R⁶, O(CO)NR⁵R⁶, C₀₋₆alkyl(SO₂)NR⁵R⁶, OC₂₋₆alkyl(SO₂)NR⁵R⁶, C₀₋₆alkylNR⁵(SO₂)R⁶, OC₂₋₆alkylNR⁵(SO₂)R⁶, SO₃R⁵, C₁₋₆alkylNR⁵(SO₂)NR⁵R⁶, OC₂₋₆alkyl(SO₂)R⁵, C₀₋₆alkyl(SO₂)R⁵, C₀₋₆alkyl(SO)R⁵, OC₂₋₆alkyl(SO)R⁵ and a 5- or 6-membered ring containing atoms independently selected from the group consisting of C, N, O and S;

R⁵ and R⁶ are independently selected from, H, C₁₋₆alkyl, C₃₋₇cycloalkyl and aryl;

m is selected from 0, 1, 2, 3 or 4;

n is selected from 0, 1, 2, 3 or 4;

p is selected from 0, 1, 2, 3 or 4; and

a salt or hydrate thereof.

In a further aspect of the invention there is provided pharmaceutical compositions comprising a therapeutically effective amount of a compound of formula I and a pharmaceutically acceptable diluent, excipients and/or inert carrier.

In yet a further aspect of the invention there is provided a pharmaceutical composition comprising a compound of formula I for use in the treatment of mGluR5 receptor mediated disorders, and for use in the treatment of neurological disorders, psychiatric disorders, gastrointestinal disorders and pain disorders.

In still a further aspect of the invention there is provided the compound of formula I for use in therapy, especially for the treatment of mGluR5 receptor mediated disorders, and for the treatment of neurological disorders, psychiatric disorders, gastrointestinal disorders and pain disorders.

A further aspect of the invention is the use of a compound according to formula X for the manufacture of a medicament for the treatment or prevention of obesity and obesity related conditions, as well as treating eating disorders by inhibition of excessive food intake and the resulting obesity and complications associated therewith.

In another aspect of the invention there is provided processes for the preparation of compounds of formula I and the intermediates used in the preparation thereof.

These and other aspects of the present invention are described in greater detail herein below.

### DETAILED DESCRIPTION OF THE INVENTION

The object of the present invention is to provide compounds exhibiting an activity at metabotropic glutamate receptors (mGluRs), especially at the mGluR5 receptors.

Listed below are definitions of various terms used in the specification and claims to describe the present invention.

For the avoidance of doubt it is to be understood that where in this specification a group is qualified by ‘hereinbefore defined’, ‘defined hereinbefore’ or ‘defined above’ said group encompasses the first occurring and broadest definition as well as each and all of the other definitions for that group.

For the avoidance of doubt it is to be understood that in this specification ‘C₁₋₆’ means a carbon group having 1, 2, 3, 4, 5 or 6 carbon atoms. Similarly ‘C₁₋₃’ means a carbon group having 1, 2, or 3 carbon atoms

In the case where a subscript is the integer 0 (zero) the group to which the subscript refers indicates that the group is absent.

In this specification, unless stated otherwise, the term “alkyl” includes both straight and branched chain alkyl groups and may be, but are not limited to methyl, ethyl, n-propyl, i-propyl, n-butyl, i-butyl, s-butyl, t-butyl, n-pentyl, i-pentyl, t-pentyl, neo-pentyl, n-hexyl or i-hexyl, t-hexyl. The term C₁₋₃alkyl has 1 to 3 carbon atoms and may be methyl, ethyl, n-propyl or i-propyl.

In this specification, unless stated otherwise, the term “cycloalkyl” refers to an optionally substituted, saturated cyclic hydrocarbon ring system. The term “C₃₋₇cycloalkyl” may be cyclopropyl, cyclobutyl, cyclopentyl, cyclohexyl or cycloheptyl.

In this specification, unless stated otherwise, the term “alkoxy” includes both straight or branched alkoxy groups. C₁₋₃alkoxy may be, but is not limited to methoxy, ethoxy, n-propoxy or i-propoxy.

In this specification, unless stated otherwise, the term “bond” may be a saturated or unsaturated bond.

In this specification, unless stated otherwise, the term “halo” and “halogen” may be fluoro, chloro, bromo or iodo.

In this specification, unless stated otherwise, the term “alkylhalo” means an alkyl group as defined above, which is substituted with halo as described above. The term “C₁₋₆alkylhalo” may include, but is not limited to fluoromethyl, difluoromethyl, trifluoromethyl, fluoroethyl, difluoroethyl or bromopropyl. The term “OC₁₋₆alkylhalo” may include, but is not limited to fluoromethoxy, difluoromethoxy, trifluoromethoxy, fluoroethoxy or difluoroethoxy.

In this specification, unless stated otherwise, the term “alkenyl” includes both straight and branched chain alkenyl groups. The term “C₂₋₆alkenyl” refers to an alkenyl group having 2 to 6 carbon atoms and one or two double bonds, and may be, but is not limited to vinyl, allyl, propenyl, i-propenyl, butenyl, i-butenyl, crotyl, pentenyl, i-pentenyl and hexenyl.

In this specification, unless stated otherwise, the term “alkynyl” includes both straight and branched chain alkynyl groups. The term C₂₋₆alkynyl having 2 to 6 carbon atoms and one or two triple bonds, and may be, but is not limited to ethynyl, propargyl, butynyl, i-butynyl, pentynyl, i-pentynyl and hexynyl.

In this specification unless otherwise stated the term “aryl” refers to an optionally substituted monocyclic or bicyclic hydrocarbon ring system containing at least one unsaturated aromatic ring. Examples and suitable values of the term “aryl” are phenyl, naphthyl, 1,2,3,4-tetrahydronaphthyl, indyl and indenyl.

In this specification, unless stated otherwise, the term “heteroaryl” refers to an optionally substituted monocyclic or bicyclic unsaturated, ring system containing at least one heteroatom selected independently from N, O or S. Examples of “heteroaryl” may be, but are not limited to thiophene, thienyl, pyridyl, thiazolyl, furyl, pyrrolyl, triazolyl, imidazolyl, oxadiazolyl, oxazolyl, isoxazolyl, pyrazolyl, imidazolonyl, oxazolonyl, thiazolonyl, tetrazolyl and thiadiazolyl, benzoimidazolyl, benzooxazolyl, tetrahydrotriazolopyridyl, tetrahydrotriazolopyrimidinyl, benzofuryl, indolyl, isoindolyl, pyridonyl, pyridazinyl, pyrimidinyl, imidazopyridyl, oxazolopyridyl, thiazolopyridyl, pyridyl, imidazopyridazinyl, oxazolopyridazinyl, thiazolopyridazinyl and purinyl.

In this specification, unless stated otherwise, the term “alkylaryl”, “alkylheteroaryl” and “alkylcycloalkyl” refer to a substituent that is attached via the alkyl group to an aryl, heteroaryl and cycloalkyl group.

In this specification, unless stated otherwise, the term “heterocycloalkyl” refers to an optionally substituted, saturated cyclic hydrocarbon ring system wherein one or more of the carbon atoms are replaced with heteroatom. The term “heterocycloalkyl” includes but is not limited to pyrrolidine, tetrahydrofuran, tetrahydrothiophene, piperidine, piperazine, morpholine, thiomorpholine, tetrahydropyran, tetrahydrothiopyran.

In this specification, unless stated otherwise the term “5- or 6-membered ring containing atoms independently selected from C, N, O or S”, includes aromatic and heteroaromatic rings as well as carbocyclic and heterocyclic rings, which may be saturated, partially saturated or unsaturated. Examples of such rings may be, but are not limited to furyl, isoxazolyl, isothiazolyl, oxazolyl, pyrazinyl, pyrazolyl, pyridazinyl, pyridyl, pyrimidyl, pyrrolyl, thiazolyl, thienyl, imidazolyl, imidazolidinyl, imidazolinyl, triazolyl, morpholinyl, piperazinyl, piperidyl, piperidonyl, pyrazolidinyl, pyrazolinyl, pyrrolidinyl, pyrrolinyl, tetrahydropyranyl, thiomorpholinyl, phenyl, cyclohexyl, cyclopentyl and cyclohexenyl.

In this specification, unless stated otherwise, the term “═NR⁵” and “═NOR⁵” include imino- and oximo-groups carrying an R⁵ substituent and may be, or be part of, groups including, but not limited to iminoalkyl, iminohydroxy, iminoalkoxy, amidine, hydroxyamidine and alkoxyamidine.

In the case where a subscript is the integer 0 (zero) the group to which the subscript refers, indicates that the group is absent, i.e. there is a direct bond between the groups. In this specification unless stated otherwise the term “fused rings” refers to two rings which share 2 common atoms.

In this specification, unless stated otherwise, the term “bridge” means a molecular fragment, containing one or more atoms, or a bond, which connects two remote atoms in a ring, thus forming either bi- or tricyclic systems.

One embodiment of the invention relates to compounds of Formula I

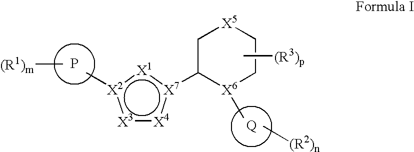

wherein

P is selected from aryl and heteroaryl;

R¹ is attached to P via a carbon atom on ring P and is selected from the group consisting of: hydroxy, halo, nitro, C₁₋₆alkylhalo, OC₁₋₆alkylhalo, C₁₋₆alkyl, OC₁₋₆alkyl, C₂₋₆alkenyl, OC₂₋₆alkenyl, C₂₋₆alkynyl, OC₂₋₆alkynyl, C₀₋₆alkylC₃₋₆cycloalkyl, OC₀₋₆alkylC₃₋₆cycloalkyl, C₀₋₆alkylaryl, OC₀₋₆alkylaryl, CHO, (CO)R⁵, O(CO)R⁵, O(CO)OR⁵, O(CNR⁵)OR⁵, C₁₋₆alkylOR⁵, OC₂₋₆alkylOR⁵, C₁₋₆alkyl(CO)R⁵, OC₁₋₆alkyl(CO)R⁵, C₀₋₆alkylCO₂R⁵, OC₁₋₆alkylCO₂R⁵, C₀₋₆alkylcyano, OC₂₋₆alkylcyano, C₀₋₆alkylNR⁵R⁶, OC₂₋₆alkylNR⁵R⁶, C₁₋₆alkyl(CO)NR⁵R⁶, OC₁₋₆alkyl(CO)NR⁵R⁶, C₀₋₆alkylNR⁵(CO)R⁶, OC₂₋₆alkylNR⁵(CO)R⁶, C₀₋₆alkylNR⁵(CO)NR⁵R⁶, C₀₋₆alkylSR⁵, OC₂₋₆alkylSR⁵, C₀₋₆alkyl(SO)R⁵, OC₂₋₆alkyl(SO)R⁵, C₀₋₆alkylSO₂R⁵, OC₂₋₆alkylSO₂R⁵, C₀₋₆alkyl(SO₂)NR⁵R⁶, OC₂₋₆alkyl(SO₂)NR⁵R⁶, C₀₋₆alkylNR⁵(SO₂)R⁶, OC₂₋₆alkylNR⁵(SO₂)R⁶, C₀₋₆alkylNR⁵(SO₂)NR⁵R⁶, OC₂₋₆alkylNR⁵(SO₂)NR⁵R⁶, (CO)NR⁵R⁶, O(CO)NR⁵R⁶, NR⁵OR⁶, C₀₋₆alkylNR⁵(CO)OR⁶, OC₂₋₆alkylNR⁵(CO)OR⁶, SO₃R⁵ and a 5- or 6-membered ring containing atoms independently selected from the group consisting of C, N, O and S;

X¹ is selected from the group consisting of: N, NR⁴ and CR⁴;

X² is selected from the group consisting of: C and N;

X³ is selected from the group consisting of: CR⁴, N and O;

X⁴ is selected from the group consisting of: CR⁴, N, NR⁴ and O;

X⁵ is selected from the group consisting of: a bond, CR⁴R^{4′}, NR⁴, O, S, SO and SO₂;

X⁶ is selected from the group consisting of: CR⁴ and N;

X⁷ is selected from the group consisting of: C and N;

R⁴ is independently selected from a group consisting of hydrogen, hydroxy, C₁₋₆alkyl, C₀₋₆alkylcyano, oxo, ═NR⁵, ═NOR⁵, C₁₋₄alkylhalo, halo, C₃₋₇cycloalkyl, O(CO)C₁₋₄alkyl, C₁₋₄alkyl(SO)C₀₋₄alkyl, C₁₋₄alkyl(SO₂)C₀₋₄alkyl, (SO)C₀₋₄alkyl, (SO₂)C₀₋₄alkyl, OC₁₋₄alkyl, C₁₋₄alkylOR⁵ and C₀₋₄alkylNR⁵R⁶;

Q is selected the group consisting of heterocycloalkyl and heteroaryl;

R² and R³ are independently selected from the group consisting of: hydroxy, C₀₋₆alkylcyano, oxo, ═NR⁵, ═NOR⁵, C₁₋₄alkylhalo, halo, C₁₋₆alkyl, C₃₋₆cycloalkyl, C₀₋₆alkylaryl, C₀₋₆alkylheteroaryl, C₁₋₆alkylcycloalkyl, C₀₋₆alkylheterocycloalkyl, OC₁₋₄alkyl, OC₀₋₆alkylaryl, O(CO)C₁₋₄alkyl, (CO)OC₁₋₄alkyl, C₀₋₄alkyl(S)C₀₋₄alkyl, C₁₋₄alkyl(SO)C₀₋₄alkyl, C₁₋₄alkyl(SO₂)C₀₋₄alkyl, (SO)C₀₋₄alkyl, (SO₂)C₀₋₄alkyl, C₁₋₄alkylOR⁵, C₀₋₄alkylNR⁵R⁶ and a 5- or 6-membered ring containing atoms independently selected from C, N, O and S, which ring may optionally be fused with a 5- or 6-membered ring containing atoms independently selected from the group consisting of C, N and O and wherein said ring and said fused ring may be substituted by one or more A;

wherein any C₁₋₆alkyl, aryl, or heteroaryl defined under R¹, R² and R³ may be substituted by one or more A;

A is selected from the group consisting of: hydrogen, hydroxy, halo, nitro, oxo, C₀₋₆alkylcyano, C₀₋₄alkylC₃₋₆cycloalkyl, C₁₋₆alkyl, —OC₁₋₆alkyl, C₁₋₆alkylhalo, OC₁₋₆alkylhalo, C₂₋₆alkenyl, C₀₋₃alkylaryl, C₀₋₆alkylOR⁵, OC₂₋₆alkylOR⁵, C₀₋₆alkylSR⁵, OC₂₋₆alkylSR⁵, (CO)R⁵, O(CO)R⁵, OC₂₋₆alkylcyano, OC₁₋₆alkylCO₂R⁵, O(CO)OR⁵, OC₁₋₆alkyl(CO)R⁵, C₁₋₆alkyl(CO)R⁵, NR⁵OR⁶, C₀₋₆NR⁵R⁶, OC₂₋₆alkylNR⁵R⁶, C₀₋₆alkyl(CO)NR¹R⁶, OC₁₋₆alkyl(CO)NR⁵R⁶, OC₂₋₆alkylNR⁵(CO)R⁶, C₀₋₆alkylNR⁵(CO)R⁶, C₀₋₆alkylNR⁵(CO)NR⁵R⁶, O(CO)NR⁵R⁶, C₀₋₆alkyl(SO₂)NR⁵R⁶, OC₂₋₆alkyl(SO₂)NR⁵R⁶, C₀₋₆alkylNR⁵(SO₂)R⁶, OC₂₋₆alkylNR⁵(SO₂)R⁶, SO₃R⁵, C₁₋₆alkylNR⁵(SO₂)NR⁵R⁶, OC₂₋₆alkyl(SO₂)R⁵, C₀₋₆alkyl(SO₂)R⁵, C₀₋₆alkyl(SO)R⁵, OC₂₋₆alkyl(SO)R⁵ and a 5- or 6-membered ring containing atoms independently selected from the group consisting of C, N, O and S;

R⁵ and R⁶ are independently selected from, H, C₁₋₆alkyl, C₃₋₇cycloalkyl and aryl;

m is selected from 0, 1, 2, 3 or 4;

n is selected from 0, 1, 2, 3 or 4;

p is selected from 0, 1, 2, 3 or 4; and

a salt or hydrate thereof.

Another embodiment the invention relates to the compounds:
4-(5-{2-[5-(3-Chloro-phenyl)-isoxazol-3-yl]-piperidin-1-yl}-4-methyl-4H [1,2,4]triazol-3-yl)-pyridine,
3-[5-(3-Chloro-phenyl)-isoxazol-3-yl]-4-(4-methyl-5-pyridin-4-yl-4H-[1,2,4]triazol-3-yl)-morpholine,
3-[5-(3-Chloro-phenyl)-isoxazol-3-yl]-4-[5-(4-difluoromethoxy-phenyl)-4-methyl-4H-[1,2,4]triazol-3-yl]-morpholine,
3-[3-(3-Chloro-phenyl)-[1,2,4]oxadiazol-5-yl]-4-(4-methyl-5-pyridin-4-yl-4H-[1,2,4]triazol-3-yl)-morpholine,
3-[3-(3-Chloro-phenyl)-[1,2,4]oxadiazol-5-yl]-4-[5-(4-difluoromethoxy-phenyl)-4-methyl-4H-[1,2,4]triazol-3-yl]-morpholine,
3-[3-(3-Chloro-phenyl)-[1,2,4]oxadiazol-5-yl]-4-(4-methyl-5-pyridin-4-yl-4H-[1,2,4]triazol-3-yl)-piperazine-1-carboxylic acid tert-butyl ester,
2-[3-(3-Chloro-phenyl)-[1,2,4]oxadiazol-5-yl]-1-(4-methyl-5-pyridin-4-yl-4H-1,2,4]triazol-3-yl)-piperazine,
2-[3-(3-Chloro-phenyl)-[1,2,4]oxadiazol-5-yl]-4-methyl-1-(4-methyl-5-pyridin-4-yl-4H-[1,2,4]triazol-3-yl)-piperazine,
3-[3-(3-Chloro-phenyl)-[1,2,4]oxadiazol-5-yl]-4-[5-(4-difluoromethoxy-phenyl)-4-methyl-4H-[1,2,4]triazol-3-yl]-piperazine-1-carboxylic acid tert-butyl ester,
2-[3-(3-Chloro-phenyl)-[1,2,4]oxadiazol-5-yl]-1-[5-(4-difluoromethoxy-phenyl)-4-methyl-4H-[1,2,4]triazol-3-yl]-piperazine,
2-[3-(3-Chloro-phenyl)-[1,2,4]oxadiazol-5-yl]-1-[5-(4-difluoromethoxy-phenyl)-4-methyl-4H-[1,2,4]triazol-3-yl]-4-methyl-piperazine,
2-[2-(3-Chlorophenyl)-2H-tetrazol-5-yl]-1-{5-[4-(difluoromethoxy)phenyl]-4-methyl-4H-1,2,4-triazol-3-yl}piperidine,
4-(5-{2-[2-(3-chlorophenyl)-2H-tetrazol-5-yl]piperidin-1-yl}-4-methyl-4H-1,2,4-triazol-3-yl)pyridine,
2-[2-(3-Chlorophenyl)-2H-tetrazol-5-yl]1-[5-(4-methoxyphenyl)-4-methyl-4H-1,2,4-triazol-3-yl]piperidine,
[4-(5-{2-[2-(3-chlorophenyl)-2H-tetrazol-5-yl]piperidin-1-yl}-4-methyl-4H-1,2,4-triazol-3-yl)phenyl]dimethylamine,
[4-(5-{2-[2-(3-Chloro-phenyl)-2H-tetrazol-5-yl]-piperidin-1-yl}-4-methyl-4H-[1,2,4]triazol-3-yl)-benzyl]-dimethyl-amine,
{2-[4-(5-{2-[2-(3-Chloro-phenyl)-2H-tetrazol-5-yl]-piperidin-1-yl}-4-methyl-4H-[1,2,4]triazol-3-yl)-phenoxy]-ethyl}-dimethyl-amine,
(R)-3-[3-(3-Chloro-phenyl)-[1,2,4]oxadiazol-5-yl]-4-(4-methyl-5-pyridin-4-yl-4H-[1,2,4]triazol-3-yl)-morpholine,
(S) 3-[3-(3-Chloro-phenyl)-[1,2,4]oxadiazol-5-yl]-4-(4-methyl-5-pyridin-4-yl-4H-[1,2,4]triazol-3-yl)-morpholine,
(R)-2-[2-(3-Chlorophenyl)-2H-tetrazol-5-yl]-1-{5-[4-(difluoromethoxy)phenyl]-4-methyl-4H-1,2,4-triazol-3-yl}piperidine,
(S)-2-[2-(3-Chlorophenyl)-2H-tetrazol-5-yl]-1-{5-[4-(difluoromethoxy)phenyl]-4-methyl-4H-1,2,4-triazol-3-yl}piperidine,
(R)-4-(5-{2-[2-(3-Chlorophenyl)-2H-tetrazol-5-yl]piperidin-1-yl}-4-methyl-4H-1,2,4-triazol-3-yl)pyridine,
(S)-4-(5-{2-[2-(3-Chlorophenyl)-2H-tetrazol-5-yl]piperidin-1-yl}-4-methyl-4H-1,2,4-triazol-3-yl)pyridine,
4-[5-(5-{2-[5-(3-Chloro-phenyl)-isoxazol-3-yl]-pyrrolidin-1-yl}-4-cyclopropyl-4H-[1,2,4]triazol-3-yl)-pyridin-2-yl]-morpholine,
4-[5-(5-{2-[5-(3-Chloro-phenyl)-isoxazol-3-yl]-pyrrolidin-1-yl}-4-methyl-4H-[1,2,4]triazol-3-yl)-pyridin-2-yl]-morpholine,
3-(5-{2-[5-(3-Chloro-phenyl)-isoxazol-3-yl]-pyrrolidin-1-yl}-4-methyl-4H-[1,2,4]triazol-3-yl)-pyridine,
4-(5-{2-[5-(3-Chloro-phenyl)-isoxazol-3-yl]-pyrrolidin-1-yl}-4-cyclopropyl-4H-[1,2,4]triazol-3-yl)-pyridine,
3-[5-(3-Chloro-phenyl)-[1,2,4]oxadioazol-3-yl]-4-(5-pyridin-4-yl-4H-[1,2,4]triazol-3-yl)-morpholine,
3-[5-(3-chlorophenyl)isoxazol-3-yl]-4-(4-cyclopropyl-5-pyridin-3-yl-4H-1,2,4-triazol-3-yl)morpholine,
3-[5-(3-chlorophenyl)isoxazol-3-yl]-4-(4-cyclopropyl-5-pyridin-4-yl-4H-1,2,4-triazol-3-yl)morpholine,
3-[5-(3-chlorophenyl)isoxazol-3-yl]-4-(4-methyl-5-pyridin-3-yl-4H-1,2,4-triazol-3-yl)morpholine,
3-[5-(3-Chloro-phenyl)-isoxazol-3-yl]-4-[5-(6-methoxy-pyridin-3-yl)-4-methyl-4H-[1,2,4]triazol-3-yl]-morpholine,
3-[3-(3-chlorophenyl)-1,2,4-oxadiazol-5-yl]-4-[5-(2-methoxypyridin-4-yl)-4-methyl-4H-1,2,4-triazol-3-yl]morpholine,
3-[3-(3-chlorophenyl)-1,2,4-oxadiazol-5-yl]-4-[5-(2-methylpyridin-4-yl)-4-methyl-4H-1,2,4-triazol-3-yl]morpholine,
3-[3-(3-chlorophenyl)-1,2,4-oxadiazol-5-yl]-4-[5-(5-fluoropyridin-3-yl)-4-methyl-4H-1,2,4-triazol-3-yl]morpholine,
3-[5-(3-chlorophenyl)isoxazol-3-yl]-4-[5-(5-fluoropyridin-3-yl)-4-methyl-4H-1,2,4-triazol-3-yl]morpholine,
3-[3-(3-chlorophenyl)-1,2,4-oxadiazol-5-yl]-4-(4-methyl-5-pyridin-2-yl-4H-1,2,4-triazol-3-yl)morpholine,
4-[5-(5-fluoropyridin-3-yl)-4-methyl-4H-1,2,4-triazol-3-yl]-3-[3-(3-iodophenyl)-1,2,4-oxadiazol-5-yl]morpholine,
3-[3-(3-iodophenyl)-1,2,4-oxadiazol-5-yl]-4-(4-methyl-5-pyridin-4-yl-4H-1,2,4-triazol-3-yl)morpholine,
3-[5-(3-chlorophenyl)isoxazol-3-yl]-4-[5-(2-methylpyridin-4-yl)-4-methyl-4H-1,2,4-triazol-3-yl]morpholine,
3-[2-(3-chlorophenyl)-2H-tetrazol-5-yl]-4-(4-methyl-5-pyridin-3-yl-4H-1,2,4-triazol-3-yl)morpholine, and
3-[2-(3-chlorophenyl)-2H-tetrazol-5-yl]-4-[5-(3,5-difluorophenyl)-4-methyl-4H-1,2,4-triazol-3-yl]morpholine.

This invention relates to polycyclic compounds of formula 1 having a variable P. In embodiments of the invention P is aryl. In particular embodiments of the invention P is phenyl.

In embodiments of the invention m is 1 or 2.

In particular embodiments of the invention P is phenyl having one or two substituents R¹. In more particular embodiments of the invention, when there is one substituent R¹ the substituent in located at the 3-position of the phenyl relative to X². In other particular embodiments of the invention when there are two substituents R¹, the substituents are located at the 2- and 5-positions of the phenyl, relative to X².

In another embodiment of the invention R¹ is selected from the group consisting of: hydrogen, halo, C₁₋₆alkylhalo, OC₁₋₆alkylhalo, C₁₋₆alkyl, OC₁₋₆alkyl, C₁₋₆alkylOR⁵, C₀₋₆alkylcyano, C₀₋₆alkylNR⁵R⁶. In still another embodiment of the invention R¹ is selected from the group consisting Cl, F, Me, OMe, CF₃, OCF₃, and CN. In yet another embodiment of the invention R¹ is Cl.

In embodiments of the invention X⁷ is C. In other embodiments of the invention X² is C. In preferred embodiments of the invention at least one of X² and X⁷ is C.

In another embodiment of the invention X³ is selected from N and O.

The invention further relates to compounds of Formula I wherein X² is C. Embodiments of the invention include those where X¹ is N or CR⁴. In a further embodiment of the invention when X³ is O, X⁴ is N and when X³ is N, X⁴ is O.

In another embodiment of the invention X² is N. In a further embodiment of the invention X¹ is N. In still a further embodiment of the invention X³ is N and X⁴ are N or CR⁴.

In another embodiment of the invention X⁵ is selected from the group consisting of CR⁴R^{4′}, NR⁴, O, S, SO and SO₂. In a further embodiment of the invention X⁵ is selected from the group consisting of CR⁴R^{4′}, NR⁴ and O. In yet a further embodiment of the invention X⁵ is selected from the group consisting of O and NR⁴.

Particular embodiments of the invention include those where the ring containing X¹, X², X³ and X⁴ are selected such that the ring formed is a tetrazole, triazole, oxadiazole, oxazole, isoxazole, or imidazole ring. Preferably the ring is tetrazole, oxadiazole or isoxazole.

In embodiments of the invention X⁶ is N. In further embodiments of the invention X⁵ is selected from O and NR⁴. In still further embodiments of the invention X⁵ is selected from CR⁴R^{4′}.

In particular embodiments of the invention when the ring containing X¹, X², X³ and X⁴ is tetrazole, X⁶ is N and X⁵ is CR⁴R^{4′}. In another particular embodiment of the invention when the ring containing X¹, X², X³ and X⁴ is selected from, oxadiazole and isoxazole, X⁶ is N and X⁵ is selected from O and NR⁴.

In another embodiment of the invention R⁴ and R^{4′} are independently selected from the group consisting of: hydrogen, C₁₋₆alkyl, C₁₋₆alkylhalo and halo.

The present invention relates to compounds of formula 1 have a ring Q. Embodiments of the invention include those where Q is heteroaryl. In preferred embodiments Q is selected from the group consisting of:

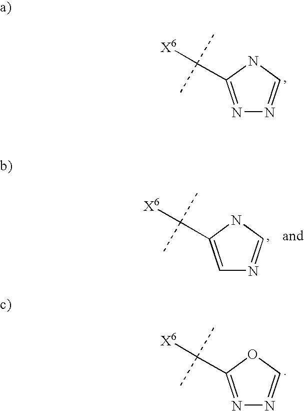

In a more preferred embodiment of the invention the ring Q is

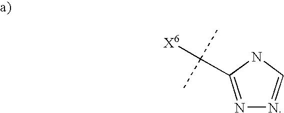

Embodiments of the invention include those where R¹ and R² are selected from the group consisting of: hydrogen, C₁₋₄alkylhalo, C₁₋₆alkyl, C₃₋₆cycloalkyl, C₀₋₆alkylaryl and C₀₋₆alkylheteroaryl.

In still another embodiment of the invention the variable any C₁₋₆alkyl, aryl, or heteroaryl defined under R¹, R² and R³ may be substituted by one or more substituents A. Particular embodiments of the invention include those where A is selected from the group consisting of: hydrogen, hydroxyl, halo, C₀₋₆alkylcyano, C₁₋₆alkyl, —OC₁₋₆alkyl, C₁₋₆alkylhalo, OC₁₋₆alkylhalo.

Embodiments of the invention include salt forms of the compounds of Formula I. Salts for use in pharmaceutical compositions will be pharmaceutically acceptable salts, but other salts may be useful in the production of the compounds of Formula I.

A suitable pharmaceutically acceptable salt of the compounds of the invention is, for example, an acid-addition salt, for example an inorganic or organic acid. In addition, a suitable pharmaceutically acceptable salt of the compounds of the invention is an alkali metal salt, an alkaline earth metal salt or a salt with an organic base.

Other pharmaceutically acceptable salts and methods of preparing these salts may be found in, for example, Remington's Pharmaceutical Sciences (18^{th} Edition, Mack Publishing Co.) 1990.

Some compounds of formula I may have chiral centres and/or geometric isomeric centres (E- and Z-isomers), and it is to be understood that the invention encompasses all such optical, diastereoisomeric and geometric isomers. The invention also relates to any and all tautomeric forms of the compounds of Formula I. The invention further relates to hydrate and solvate forms of the compounds of Formula I.

Pharmaceutical Composition

According to one aspect of the present invention there is provided a pharmaceutical composition comprising as active ingredient a therapeutically effective amount of the compound of Formula I, or salts, solvates or solvated salts thereof, in association with one or more pharmaceutically acceptable diluent, excipients and/or inert carrier.

The composition may be in a form suitable for oral administration, for example as a tablet, pill, syrup, powder, granule or capsule, for parenteral injection (including intravenous, subcutaneous, intramuscular, intravascular or infusion) as a sterile solution, suspension or emulsion, for topical administration e.g. as an ointment, patch or cream or for rectal administration e.g. as a suppository.

In general the above compositions may be prepared in a conventional manner using one or more conventional excipients, pharmaceutical acceptable diluents and/or inert carriers.

Suitable daily doses of the compounds of formula I in the treatment of a mammal, including man are approximately 0.01 to 250 mg/kg bodyweight at peroral administration and about 0.001 to 250 mg/kg bodyweight at parenteral administration.

The typical daily dose of the active ingredients varies within a wide range and will depend on various factors such as the relevant indication, severity of the illness being treated, the route of administration, the age, weight and sex of the patient and the particular compound being used, and may be determined by a physician.

Medical Use

It has been found that the compounds according to the present invention, exhibit a high degree of potency and selectivity for individual metabotropic glutamate receptor (mGluR) subtypes. Accordingly, the compounds of the present invention are expected to be useful in the treatment of conditions associated with excitatory activation of mGluR5 and for inhibiting neuronal damage caused by excitatory activation of mGluR5. The compounds may be used to produce an inhibitory effect of mGluR5 in mammals, including man.

The mGluR Group I receptor including mGluR5 are highly expressed in the central and peripheral nervous system and in other tissues. Thus, it is expected that the compounds of the invention are well suited for the treatment of mGluR5-mediated disorders such as acute and chronic neurological and psychiatric disorders, gastrointestinal disorders, and chronic and acute pain disorders.

The invention relates to compounds of Formula I, as defined hereinbefore, for use in therapy.

The invention relates to compounds of Formula I, as defined hereinbefore, for use in treatment of mGluR5-mediated disorders.

The invention relates to compounds of Formula I, as defined hereinbefore, for use in treatment of Alzheimer's disease senile dementia, AIDS-induced dementia, Parkinson's disease, amylotropic lateral sclerosis, Huntington's Chorea, migraine, epilepsy, schizophrenia, depression, anxiety, acute anxiety, ophthalmological disorders such as retinopathies, diabetic retinopathies, glaucoma, auditory neuropathic disorders such as tinnitus, chemotherapy induced neuropathies, post-herpetic neuralgia and trigeminal neuralgia, tolerance, dependency, Fragile X, autism, mental retardation, schizophrenia and Down's Syndrome.

The invention relates to compounds of Formula I, as defined hereinbefore, for use in treatment of pain related to migraine, inflammatory pain, neuropathic pain disorders such as diabetic neuropathies, arthritis and rheumatoid diseases, low back pain, post-operative pain and pain associated with various conditions including angina, renal or biliary colic, menstruation, migraine and gout.

The invention relates to compounds of Formula I as defined hereinbefore, for use in treatment of stroke, head trauma, anoxic and ischemic injuries, hypoglycemia, cardiovascular diseases and epilepsy.

The present invention relates also to the use of a compound of Formula I as defined hereinbefore, in the manufacture of a medicament for the treatment of mGluR Group I receptor-mediated disorders and any disorder listed above.

One embodiment of the invention relates to the use of a compound according to Formula I in the treatment of gastrointestinal disorders.

Another embodiment of the invention relates to the use of a compound according to Formula I, for the manufacture of a medicament for the inhibition of transient lower esophageal sphincter relaxations, for the treatment of GERD, for the prevention of G.I. reflux, for the treatment regurgitation, treatment of asthma, treatment of laryngitis, treatment of lung disease and for the management of failure to thrive.

A further embodiment of the invention is the use of a compound according to Formula I for the manufacture of a medicament for the treatment or prevention of functional gastrointestinal disorders, such as functional dyspepsia (FD). Yet another aspect of the invention is the use of a compound according to formula I for the manufacture of a medicament for the treatment or prevention of irritable bowel syndrome (IBS), such as constipation predominant IBS, diarrhea predominant IBS or alternating bowel movement predominant IBS.

₆alkyl(SO₂)R⁵, C₀₋₆alkyl(SO)R⁵, OC₂₋₆alkyl(SO)R⁵ and a 5- or 6-membered ring containing atoms independently selected from the group consisting of C, N, O and S;

R⁵ and R⁶ are independently selected from, H, C₁₋₆alkyl, C₃₋₇cycloalkyl and aryl;

m is selected from 0, 1, 2, 3 or 4;

n is selected from 0, 1, 2, 3 or 4;

p is selected from 0, 1, 2, 3 or 4; and

a salt or hydrate thereof.

In a further aspect of the invention there is provided pharmaceutical compositions comprising a therapeutically effective amount of a compound of formula I and a pharmaceutically acceptable diluent, excipients and/or inert carrier. In yet a further aspect of the invention there is provided a pharmaceutical composition comprising a compound of formula I for use in the treatment of mGluR5 receptor mediated disorders, and for use in the treatment of neurological disorders, psychiatric disorders, gastrointestinal disorders and pain disorders.

In still a further aspect of the invention there is provided the compound of formula I for use in therapy, especially for the treatment of mGluR5 receptor mediated disorders, and for the treatment of neurological disorders, psychiatric disorders, gastrointestinal disorders and pain disorders.

A further aspect of the invention is the use of a compound according to formula I for the manufacture of a medicament for the treatment or prevention of obesity and obesity related conditions, as well as treating eating disorders by inhibition of excessive food intake and the resulting obesity and complications associated therewith.

In another aspect of the invention there is provided processes for the preparation of compounds of formula I and the intermediates used in the preparation thereof. These and other aspects of the present invention are described in greater detail herein below.

The invention also provides a method of treatment of mGluR5-mediated disorders and any disorder listed above, in a patient suffering from, or at risk of, said condition, which comprises administering to the patient an effective amount of a compound of Formula I, as hereinbefore defined.

The dose required for the therapeutic or preventive treatment of a particular disorder will necessarily be varied depending on the host treated, the route of administration and the severity of the illness being treated.

In the context of the present specification, the term “therapy” and “treatment” includes prevention or prophylaxis, unless there are specific indications to the contrary. The terms “therapeutic” and “therapeutically” should be construed accordingly.

In this specification, unless stated otherwise, the term “antagonist” and “inhibitor” shall mean a compound that by any means, partly or completely, blocks the transduction pathway leading to the production of a response by the ligand.

The term “disorder”, unless stated otherwise, means any condition and disease associated with metabotropic glutamate receptor activity.

Non-Medical Use

In addition to their use in therapeutic medicine, the compounds of Formula I, salts or hydrates thereof, are also useful as pharmacological tools in the development and standardisation of in vitro and in vivo test systems for the evaluation of the effects of inhibitors of mGluR related activity in laboratory animals such as cats, dogs, rabbits, monkeys, rats and mice, as part of the search for new therapeutics agents.

Methods of Preparation

Another aspect of the present invention provides processes for preparing compounds of Formula I, or salts or hydrates thereof. Processes for the preparation of the compounds in the present invention are described herein.

Throughout the following description of such processes it is to be understood that, where appropriate, suitable protecting groups will be added to, and subsequently removed from, the various reactants and intermediates in a manner that will be readily understood by one skilled in the art of organic synthesis. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to the one skilled in the art of organic synthesis. Examples of transformations are given below, and it is to be understood that the described transformations are not limited only to the generic groups or substituents for which the transformations are exemplified. References and descriptions on other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994). Techniques for purification of intermediates and final products include for example, straight and reversed phase chromatography on column or rotating plate, recrystallisation, distillation and liquid-liquid or solid-liquid extraction, which will be readily understood by the one skilled in the art. The definitions of substituents and groups are as in formula I except where defined differently. The term “room temperature” and “ambient temperature” shall mean, unless otherwise specified, a temperature between 16 and 25° C.

The term “reflux” shall mean, unless otherwise stated, in reference to an employed solvent a temperature at or above the boiling point of named solvent.

Abbreviations

aq. Aqueous

atm atmosphere

BINAP 2,2′Bis(diphenylphosphino)-1,1′-binaphthyl

Boc, BOC tert-butoxycarbonyl

CDIN,N′-Carbonyldiimidazole

dba Dibenzylideneacetone

DCC N,N-Dicyclohexylcarbodiimide

DCM Dichloromethane

DEA N,N-Diisopropylethylamine

DIBAL-H Diisobutylaluminum hydride

DICN,N′-Diisopropylcarbodiimide

DMAP N,N-Dimethyl-4-aminopyridine

DMF Dimethylformamide

DMSO Dimethylsulfoxide

DPPF 1,1′-Bis(diphenylphosphino)ferrocene

EA or EtOAc Ethyl acetate

EDC, EDCl N-[3-(dimethylamino)propyl]-N′-ethylcarbodiimide hydrochloride

Et Ethyl

Et₂O Diethyl ether

EtI Iodoethane

EtOH Ethanol

Et₃N Triethylamine

Fmoc, FMOC 9-Fluorenylmethoxycarbonyl

h hour(s)

HBTU O-(Benzotriazol-1-yl)-N,N,N′,N′-tetramethyluronium hexafluorophosphate

HetAr Heteroaryl

HOBt N-Hydroxybenzotriazole

HPLC high performance liquid chromatography

LCMS HPLC mass spec

MCPBAm-chlorbenzoic acid

Me Methyl

MeCN Acetonitrile

MeIIodomethane

MeMgCl methyl magnesium chloride

MeOH Methanol

min Minutes

NaOAc sodium acetate

nBunormal butyl

nBuLi, n-BuLi 1-butyllithium

NCS N-chlorosuccinimide

NMR nuclear magnetic resonance

o.n. over night

OAc acetate

OMs mesylate or methane sulfonate ester

OTstosylate, toluene sulfonate or 4-methylbenzene sulfonate ester

PPTS pyridinium p-toluenesulfonate

pTsOH p-toluenesulfonic acid

RT, rt, r.t. room temperature

sat. Saturated

SPEsolid phase extraction (usually containing silica gel)

TBAF tetrabutylammonium fluoride

tBu, t-Bu tert-butyl

tBuOH, t-BuOH tert-butanol

TEA Triethylamine

THF Tetrahydrofuran

Preparation of Intermediates

The intermediates provided in synthetic paths given below, are useful for further preparation of compounds of formula I. Other starting materials are either commercially available or can be prepared via methods described in the literature. The synthetic pathways described below are non-limiting examples of preparations that can be used. One of skill in the art would understand other pathways might be used.
Synthesis of Isoxazoles

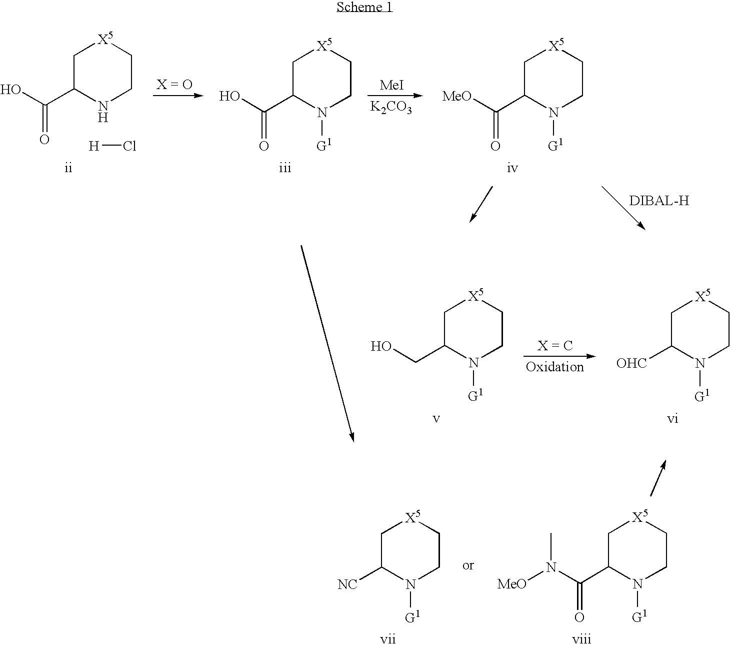

Aldehydes of formula vi wherein X⁵ is as defined in formula I may be used in the preparation of isoxazoles. Commercially available acid derivatives of formula ii wherein X⁵ is O, S, C, N—R² and N-G² (G² is a protecting group orthogonal to G¹) may undergo N-protection to yield compounds of formula iii wherein G¹ is a protecting group such as Boc or Fmoc using methods well known in the art. The acid moiety in compounds of formula iii may be transformed into an alkyl ester of formula iv, such as for example the methyl or ethyl ester, which may be transformed to aldehydes of formula vi using a mild reducing agent such as DIBAL-H in a solvent such as toluene at low temperature, for example −78° C. Higher temperatures or stronger reducing agents may result in formation of the primary alcohols of formula v, either exclusively or as a mixture with the aldehydes of formula vi. Other functional groups such as the primary alcohol in compounds of formula v, the nitrile in compounds of formula vii and Weinreb amide moiety in compounds of formula viii may be transformed into aldehydes of formula vi utilizing procedures established in the art. Additionally, acids of formula ii may be converted into nitrites of formula vii by methods known in the art, for example by conversion of the acid to the primary amide followed by dehydration to the nitrile.

Aldehydes of formula vi may be converted to oximes of formula ix by treatment with hydroxylamine, in a solvent such as pyridine, at a temperature between 0° C. to room temperature. Isoxazoles of formula x may be prepared by chlorination of oximes of formula ix using a reagent such as N-chlorosuccinimide (NCS), followed by 1,3-dipolar cycloaddition with the appropriately R-substituted acetylenes, wherein R may be (R¹)ₘ—P or a masking group which may later be converted to (R¹)ₘ—P (Steven, R. V. et al. J. Am. Chem. Soc. 1986, 108, 1039). The isoxazole intermediate x can subsequently be deprotected to give xi by standard methods.

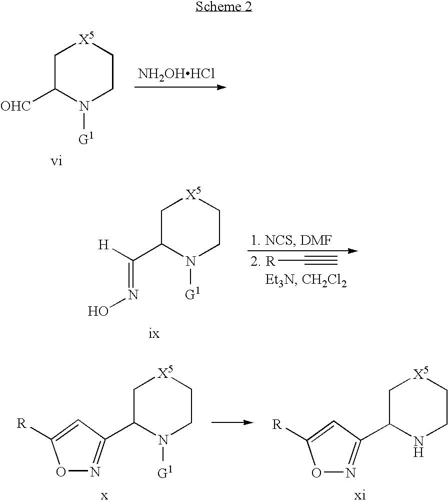

Isoxazoles of formula x wherein R is a masking group may be prepared in this manner and the masking group transformed into (R¹)ₘ—P subsequent to isoxazole ring formation. For example, the use of trialkylstannylacetylenes would result in a trialkylstannyl isoxazole which may undergo reactions such as for example Stille type cross coupling to introduce aryl substituents by coupling to an appropriate aryl halide.
Synthesis of [1,2,4]-Oxadiazoles

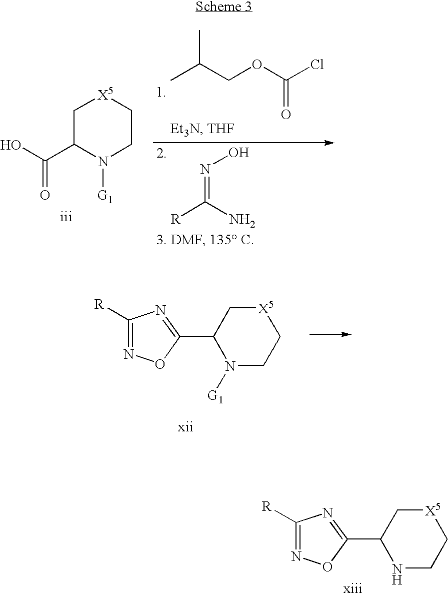

Carboxylic acids of formula iii may be used in the preparation of the corresponding 3-R substituted [1,2,4]oxadiazoles of formula xii by activation of the acid moiety, addition of a suitable R-substituted hydroxyamidine to form an ester, followed by cyclization to the oxadiazole. [See Tetrahedron Lett., 2001, 42, 1495-98, Tetrahedron Lett., 2001, 42, 1441-43, and Bioorg. Med. Chem. Lett. 1999, 9, 1869-74]. The acid may be activated as the mixed anhydride using an alkyl chloroformate such as isobutyl chloroformate, in the presence of a base such as triethylamine in a suitable solvent such as THF. Alternatively, other well known methods of activating the acid may be employed, including in situ activation of the acid using a reagent such as EDCI, DCC, DIC or HBTU, with or without the presence of co-reagents such as HOBt or DMAP, in suitable solvents such as DMF, DCM, THF, or MeCN at a temperature from −20 to 100° C. The cyclization may be accomplished by heating in a solvent such as pyridine or DMF, under microwave irradiation or by employing catalysts such as TBAF. R-substituted hydroxyamidines are available from nitrites by addition of hydroxylamine hydrochloride in the presence of a base such as NaOH, NaHCO₃ or Na₂CO₃, to generate the free hydroxylamine, in a solvent such as ethanol or methanol or the like, at temperatures between room temperature and 100° C.

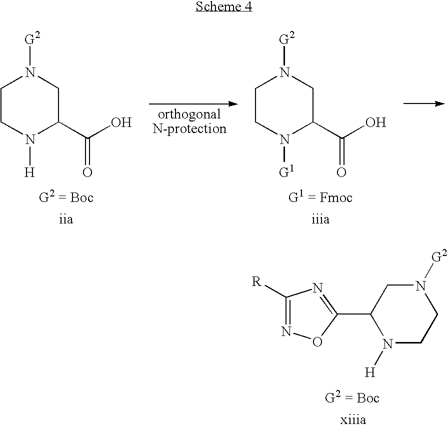

Compounds of formula ii wherein X is N-G² provides a convenient method of obtaining the free NH compound of formula I. For example, the commercially available acid derivative of formula iia wherein X is N-Boc may be orthogonally N-protected with a protecting group G¹ such as for example Fmoc. The resulting intermediate iiia may be transformed into the corresponding [1,2,4]-oxadiazoles using methods described above. When Fmoc is employed for one of the protecting groups, [1,2,4]-oxadiazole ring formation methods involving a base, such as activation with chloroformate in the presence of triethylamine or ring closure in pyridine, may effect removal of the protecting group giving xiiia directly without isolation of the 2-(3-R-[1,2,4]oxadiazol-5-yl)-piperazine intermediate.

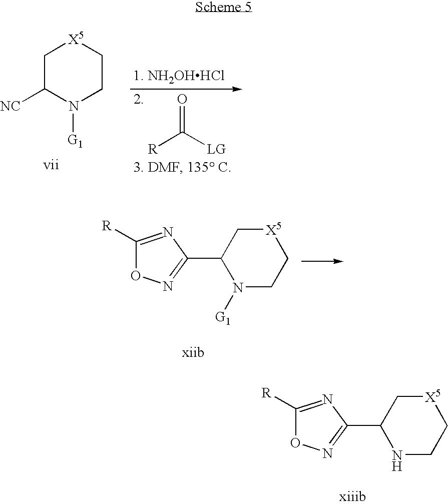

5-R substituted [1,2,4]oxadiazoles of formula xiib may be prepared from nitrites of formula vii by effectively reversing the substituents attached to the [1,2,4]-oxadiazole. Nitriles of formula vii react with hydroxylamine as described above to provide the intermediate hydroxyamidine, and may be converted to the [1,2,4]oxadiazoles of formula xiib using an acylating agent containing the R group using the method described above for conversion of compounds of formula iii to compounds of formula xii.
Synthesis of Tetrazoles

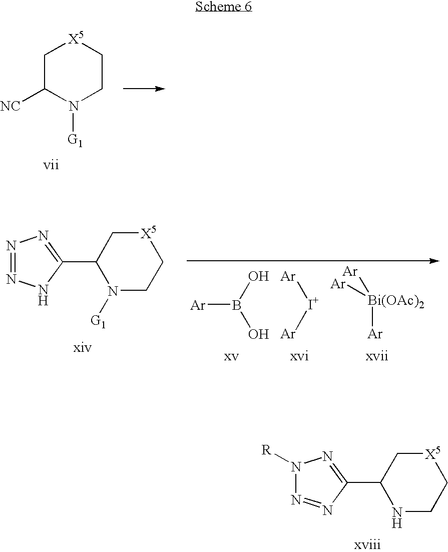

Nitriles of formula vii may be used in the preparation of the corresponding tetrazoles of formula xviii by treatment with an azide, such as NaN₃, LiN₃, trialkylyltinazide or trimethylsilylazide, preferably with a catalyst such as dibutyltin oxide or ZnBr₂, in solvents such as DMF, water or toluene at a temperature of 80 to 200° C. by conventional heating or microwave irradiation [See J. Org. Chem. 2001, 7945-7950; J. Org. Chem. 2000, 7984-7989 or J. Org. Chem. 1993, 4139-4141].

N2-arylation of 5-substituted tetrazoles have been reported in the literature using a variety of coupling partners. Compounds of formula xviii wherein R is an aryl group may be prepared using for example boronic acids of formula xv [with the B(OH)₂ moiety], or the corresponding iodonium salts of formula xvii [with the I⁺—Ar moiety], or the corresponding triarylbismuth diacetates [with the Bi(OAc)₂Ar₂ moiety], as arylating agents mediated by transition metals [See Tetrahedron Lett. 2002, 6221-6223; Tetrahedron Lett. 1998, 2941-2944; Tetrahedron Lett. 1999, 2747-2748]. With boronic acids, stoichiometric amounts of Cu(II)acetate and pyridine are used in solvents such as dichloromethane, DMF, dioxane or THF at a temperature of room temperature to 100° C. With iodonium salts, catalytic amounts of Pd(II)-compounds, such as Pd(dba)₂ or Pd(OAc)₂, together with catalytic amounts of Cu(II)-carboxylates, such as Cu(II)-phenylcyclopropylcarboxylate, and bidentate ligands, such as BINAP or DPPF, are used in solvents such as t-BuOH at a temperature of 50 to 100° C. With triarylbismuth diacetates, catalytic amounts of cupric acetate may be employed in the presence of N,N,N′,N′-tetramethylguanidine in a suitable solvent such as THF with heating at a temperature of 40-60° C. Iodonium salts of formula xvi may be obtained from, for example, the respective boronic acids by treatment with hypervalent iodine substituted aromatics, such as hydroxyl(tosyloxy)iodobenzene or PhI(OAc)₂x2TfOH, in dichloromethane or the like [See Tetrahedron Lett. 2000, 5393-5396]. Triarylbismuth diacetates may be prepared from aryl magnesium bromides with bismuth trichloride in a suitable solvent such as refluxing THF to give the triarylbismuthane, which is then oxidized to the diacetate using an oxidizing agent such as sodium perborate in acetic acid [Synth. Commun. 1996, 4569-75].
Synthesis of [1,2,3]triazoles

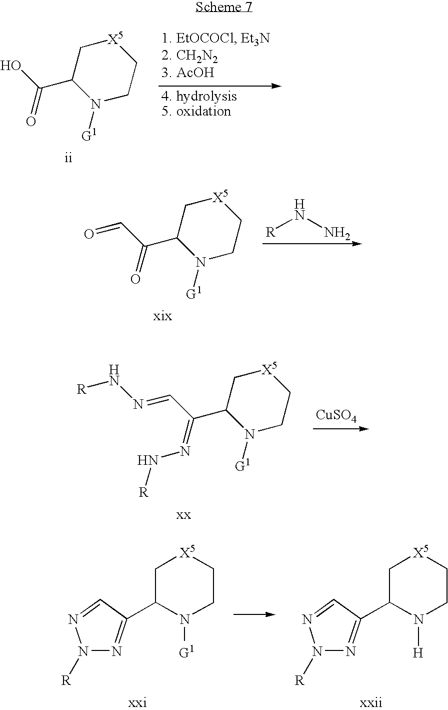

Ketoaldehydes of formula xix are available from compounds of formula ii via activation of the acid moiety, reaction with diazomethane to form an intermediate alpha-diazoketone, and trapping with an acid such as acetic acid to form an alpha-acetylated ketone intermediate, which can be converted to compounds of formula xix by hydrolysis and oxidation. [See Bioorg. Med. Chem. 2002, 10, 2199-2206] Ketoaldehydes of formula xix will react with arylhydrazines with in acetic acid and water at −20 to 120° C. to form bis-hydrazones of formula xx, which may undergo cyclization in the presence of copper (II) sulfate in aqueous mixtures of for example dioxane or THF at −20 to 120° C. to form [1,2,3]triazoles of formula xxi. [See J. Med. Chem. 1978, 21, 1254-60 and J. Org Chem. 1948, 13, 807-14] Compounds of formula xxi may be deprotected as above to yield the secondary amines of formula xxii.
Synthesis of Q Ring: Amino-Triazoles

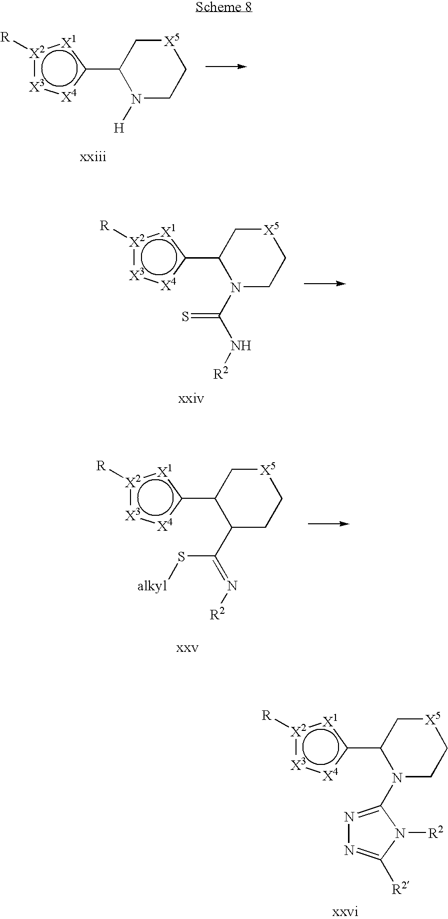

The deprotected amines of formula xi, xiii, xviii and xxii may be subjected to a sequence of thiourea formation, methylation and triazole formation to deliver compounds of formula I wherein the Q ring is a triazole attached to the newly deprotected secondary amine. Thioureas of formula xxiv are available from well established methods using for example an isothiocyanate, R²SCN, or 1,1-thiocarbonyl-diimidazole in the presence of R²NH₂, in a solvent such as methanol, ethanol and the like, at a temperature between room temperature and 100° C., and are typically carried out at 60° C. Alkylation of the thiourea intermediates can be performed using an alkylating agents such iodomethane or iodoethane, in a solvent such as DMF, acetone, CH₂Cl₂, at room temperature or elevated temperatures to give the isothiourea of formula xxv. When an iodoalkane is employed, the product may be isolated as the hydroiodide salt [See Synth. Commun. 1998, 28, 741-746]. Compounds of formula xxv may react with an acyl hydrazine or with hydrazine followed by an acylating agent to form an intermediate which may be cyclized to the 3-aminotriazoles of formula xxvi by heating at 50 to 200° C. in a suitable solvent such as pyridine or DMF.
Other Functional Group Transformations

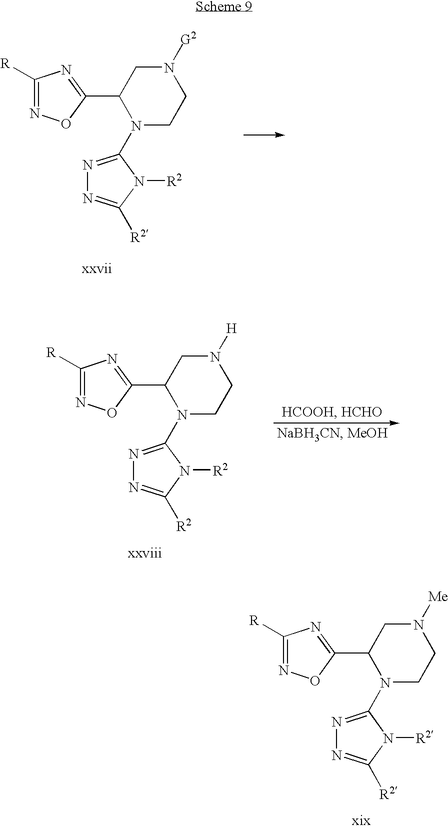

It is to be understood that when additional functional groups are present in compounds of formula I or any precursor, those functional groups may be employed to introduce other substituents or functional groups by methods established in the art when there are no other incompatible reactive sites. For example, in compounds of formula xxvii available from the orthogonally protected bisamine xiiia described above, the secondary amine obtained by deprotection of G² may undergo alkylation or reductive amination to generate a tertiary amine of formula xix. Additionally, other substituents not explicitly drawn in the schemes may be present as described in formula I providing no interference with the reactions described above is caused by said substituents.

The invention further relates to the following compounds, which may be used as intermediates in the preparation of compounds of formula I;
Methyl 4-dimethylaminomethyl-benzoate
Ethyl 4-(2-dimethylamino-ethoxy)-benzoate
4-Dimethylaminomethyl-benzoic acid hydrazide
4-(2-Dimethylamino-ethoxy)-benzoic acid hydrazide
4-Difluoromethoxy-benzoic acid hydrazide
Tris-(3-chloro-phenyl)-bismuthane
Tris-(3-chloro-phenyl)-bismuthane diacetate
2-Hydroxymethyl-piperidine-1-carboxylic acid tert-butyl ester
Morpholine-3,4-dicarboxylic acid 4-tert-butyl ester
Piperazine-1,2,4-tricarboxylic acid 4-tert-butyl ester 1-(9H-fluoren-9-ylmethyl) ester
2-Formyl-piperidine-1-carboxylic acid tert-butyl ester
Morpholine-3,4-dicarboxylic acid 4-tert-butyl ester 3-methyl ester
3-Formyl-morpholine-4-carboxylic acid tert-butyl ester
2-Cyano-piperidine-1-carboxylic acid tert-butyl ester
2-(1H-Tetrazol-5-yl)-piperidine-1-carboxylic acid tert-butyl ester
2-(Hydroxyimino-methyl)-piperidine-1-carboxylic acid tert-butyl ester
3-(Hydroxyimino-methyl)-morpholine-4-carboxylic acid tert-butyl ester
2-[5-(3-Chloro-phenyl)-isoxazol-3-yl]-piperidine-1-carboxylic acid tert-butyl ester
3-[5-(3-chloro-phenyl)-isoxazol-3-yl]-morpholine-4-carboxylic acid tert-butyl ester
3-[3-(3-Chloro-phenyl)-[1,2,4]oxadiazol-5-yl]-morpholine-4-carboxylic acid tert-butyl ester
3-[3-(3-Chloro-phenyl)-[1,2,4]oxadiazol-5-yl]-piperazine-1-carboxylic acid tert-butyl ester
2-[2-(3-chloro-phenyl)-2H-tetrazol-5-yl]-piperidine-1-carboxylic acid tert-butyl ester
2-[5-(3-Chloro-phenyl)-isoxazol-3-yl]-piperidine
3-[5-(3-Chloro-phenyl)-isoxazol-3-yl]-morpholine
3-[3-(3-Chloro-phenyl)-[1,2,4]oxadiazol-5-yl]-morpholine
2-[2-(3-Chloro-phenyl)-2H-tetrazol-5-yl]-piperidine
2-[5-(3-Chloro-phenyl)-isoxazol-3-yl]-piperidine-1-carbothioic acid methylamide
3-[5-(3-Chloro-phenyl)-isoxazol-3-yl]-morpholine-4-carbothioic acid methylamide
3-[3-(3-Chloro-phenyl)-[1,2,4]oxadiazol-5-yl]-morpholine-4-carbothioic acid methylamide
3-[3-(3-Chloro-phenyl)-[1,2,4]oxadiazol-5-yl]-4-methylthiocarbamoyl-piperazine-1-carboxylic acid tert-butyl ester
2-[2-(3-Chloro-phenyl)-2H-tetrazol-5-yl]-piperidine-1-carbothioic acid methylamide
2-[5-(3-Chloro-phenyl)-isoxazol-3-yl]-N-methyl-piperidine-1-carboximidothioic acid methyl ester
3-[5-(3-Chloro-phenyl)-isoxazol-3-yl]-N-methyl-morpholine-4-carboximidothioic acid methyl ester
3-[3-(3-Chloro-phenyl)-[1,2,4]oxadiazol-5-yl]-methylmorpholine-4-carboximidothioic acid methyl ester
3-[3-(3-Chloro-phenyl)-[1,2,4]oxadiazol-5-yl]-4-(methylimino-methylsulfanyl-methyl)-piperazine-1-carboxylic acid tert-butyl ester
2-[2-(3-Chloro-phenyl)-2H-tetrazol-5-yl]-N-methyl-piperidine-1-carboximidothioic acid methyl ester

### EXAMPLES

The invention will now be illustrated by the following non-limiting examples.

General Methods

All starting materials are commercially available or earlier described in the literature. The ¹H and ¹³C NMR spectra were recorded either on Bruker 300, Bruker DPX400 or Varian +400 spectrometers operating at 300, 400 and 400 MHz for ¹H NMR respectively, using TMS or the residual solvent signal as reference, in deuterated chloroform as solvent unless otherwise indicated. All reported chemical shifts are in ppm on the delta-scale, and the fine splitting of the signals as appearing in the recordings (s: singlet, br s: broad singlet, d: doublet, t: triplet, q: quartet, m: multiplet).

Analytical in line liquid chromatography separations followed by mass spectra detections, were recorded on a Waters LCMS consisting of an Alliance 2795 (LC) and a ZQ single quadropole mass spectrometer. The mass spectrometer was equipped with an electrospray ion source operated in a positive and/or negative ion mode. The ion spray voltage was ±3 kV and the mass spectrometer was scanned from m/z 100-700 at a scan time of 0.8 s. To the column, X-Terra MS, Waters, C8, 2.1×50 mm, 3.5 mm, was applied a linear gradient from 5% to 100% acetonitrile in 10 mM ammonium acetate (aq.), or in 0.1% TFA (aq.). Preparative reversed phase chromatography was run on a Gilson autopreparative HPLC with a diode array detector using an XTerra MS C8, 19×300 mm, 7 mm as column. Purification by a chromatotron was performed on rotating silica gel/gypsum (Merck, 60 PF-254 with calcium sulphate) coated glass sheets, with coating layer of 1, 2, or 4 mm using a TC Research 7924T chromatotron. Purification of products were also done by flash chromatography in silica-filled glass columns or in plastic SPE tubes pre-filled with silica gel.

Microwave heating was performed in a Smith Synthesizer Single-mode microwave cavity producing continuous irradiation at 2450 MHz (Personal Chemistry AB, Uppsala, Sweden).

### Example 1

### Methyl 4-dimethylaminomethyl-benzoate

Methyl 4-(bromomethyl)benzoate (4.58 g, 20 mmol) was mixed with 45% dimethylamine (5.57 mL, 2.5 mmol) in THF (50 mL) at room temperature for 30 min. The mixture was concentrated in vacuo and the residue was diluted with water and extracted with ether. The organic layer was dried with MgSO₄ and concentrated in vacuo to give the title compound (4.0 g) as pale yellow oil. ¹H NMR (CDCl₃), δ (ppm): 8.01 (d, 2H), 7.40 (d, 2H), 3.92 (s, 3H), 3.48 (s, 2H) and 2.26 (s, 6H).

### Example 2

### Ethyl 4-(2-dimethylamino-ethoxy)-benzoate

Ethyl 4-hydroxy-benzoate (16.6 g, 0.1 mol) was mixed with (2-chloro-ethyl)-dimethyl-amine hydrochloride (40 g, 0.28 mol) and K₂CO₃ (100 g, 0.724 mol) in DMF. The mixture was heated to 150° C. for 4 h, and then poured into ice-water and the product was extracted into ethyl acetate. The ethyl acetate layer was washed with brine and the product was acidified with 1N HCl (130 mL) and the ethyl acetate layer was discarded. The acidified aqueous layer was washed with ethyl acetate, then basified with 2M sodium carbonate (100 mL) and the product was extracted into ethyl acetate again. This organic layer was washed with brine, dried with MgSO₄, filtered and concentrated to give the title compound (12.6 g, 53%) as a sticky pale yellow-brown oil. ¹H NMR (CDCl₃), δ (ppm): 8.01 (d, 2H), 6.95 (d, 2H), 4.36 (q, 2H), 4.13 (t, 2H), 2.76 (t, 2H), 2.36 (s, 6H) and 1.39 (t, 3H).

### Example 3

### 4-Dimethylaminomethyl-benzoic acid hydrazide

Methyl 4-dimethylaminomethyl-benzoate (4.0 g, 20 mmol) was mixed with hydrazine hydrate (9.7 ml, 200 mmol) in methanol at 80° C. overnight. The mixture was concentrated in vacuo and the residue was triturated with ether to give the title compound (3.37 g, 84.2%) as a white solid. ¹H NMR (DMSO-d₆), δ (ppm): 9.75 (w, 1H), 7.76 (d, 2H), 7.35 (d, 2H), 4.50 (w, 2H), 3.41 (s, 2H) and 2.13 (s, 6H).

### Example 4

### 4-(2-Dimethylamino-ethoxy)-benzoic acid hydrazide

Ethyl 4-(2-dimethylamino-ethoxy)-benzoate (12.6 g, 53 mmol) was mixed with hydrazine hydride (26.5 g, 0.5 mol) in ethanol at 100° C. in a sealed flask overnight. The mixture was concentrated and triturated with ether to give the title compound (9.83 g, 82.9%) as a pale yellow solid. ¹H NMR (DMSO-d₆), δ (ppm): 9.62 (s, 1H), 7.77 (d, 2H), 6.97 (d, 2H), 4.45 (b, 2H), 4.08 (t, 2H), 2.61 (t, 2H) and 2.20 (s, 6H).

### Example 5

### 4-Difluoromethoxy-benzoic acid hydrazide

HOBt (2.2 g, 15.9 mmol) and EDCI (3.1 g, 15.9 mmol) were added to 4-difluoromethoxy-benzoic acid (2.5 g, 13.3 mmol) in acetonitrile (25 mL) at room temperature. After two hours, a solution of hydrazine monohydrate (0.493 mL, 10.2 mmol) and cyclohexane (0.33 mL) in acetonitrile (5.0 mL) was added drop-wise at 0° C. After stirring at room temperature for 2 hours, the solvent was removed in vacuo and the residue was diluted with ethyl acetate, washed saturated sodium bicarbonate (4 times), dried over sodium sulfate, filtered and concentrated to afford the title compound (2.12 g, 79%, white solid). ¹H NMR (DMSO) δ (ppm): 9.80 (bs, 1H), 7.88 (m, 2H), 7.34 (t, 1H), 7.23 (m, 2H), 4.50 (bs, 2H).

### Example 6

### Bis-(3-chloro-phenyl)-iodonium tetrafluoroborate

Bis(acetyloxy)(3-chlorophenyl)-λ-3-iodane was prepared as in literature [Kazmierczak, P.; Skulski, L., Synthesis 1998, 12, 1721-1723]. To stirred mixture of 3-chlorophenylboronic acid 0.821 g (5.25 mmol) and BF₃.Et₂O (0.78 g, 5.5 mmol) in dichloromethane (50 mL) at 0° C. was added a solution of bis(acetyloxy)(3-chlorophenyl)-λ-3-iodane (1.78 g, 5 mmol) in dichloromethane (50 mL) under argon, and the reaction mixture was stirred for 1.5 hours at 0° C. Saturated aqueous NH₄BF₄ (10.5 g, 100 mol) was added and the reaction mixture was stirred for an hour, poured into water and extracted with dichloromethane. The organic layer was concentrated to give a solid residue, which was triturated with diethyl ether to give the title compound (off-white solid, 1.70 g, 78%). ¹H NMR (CDCl₃), δ (ppm): 8.02 (m, 4H), 7.58 (dm, 2H), 7.4 (t, 2H).

### Example 7

### Copper(II) 2-phenylcyclopropanecarboxyate

Sodium hydroxide (0.81 g, 20.25 mmol) in water (10 mL) was added to 2-phenylcyclopropanecarboxyate (32.4 g, 20 mmol) and the mixture was stirred until the solid completely dissolved. A solution of copper(II) sulfate (2.44 g, 10 mmol) in water was added in a dropwise manner. The mixture was stirred for 2 h, and the pale blue precipitate was collected by filtration, dried under vacuum and used without further purification.

### Example 8

### 2-Hydroxymethyl-piperidine-1-carboxylic acid tert-butyl ester

Di-tert-butyl dicarbonate (8.3 g, 38.2 mmol) was added to a stirred solution of piperidinemethanol (4.0 g, 37.4 mmol) in CH₂Cl₂ (50 mL) and 1N NaOH (50 mL, 50 mmol) was added. The mixture was stirred at room temperature overnight. Reaction mixture was diluted with CH₂Cl₂ and the aqueous phase was separated. The aqueous phase was extracted with dichloromethane (3×30 mL). The combined organic phase was washed with water (30 mL) and brine (30 mL), dried (sodium sulfate), filtered and concentrated in-vacuo to give the crude product which was triturated with hexane to afford the title compound as white solid (4.8 g, 64%).

### Example 9

### Morpholine-3,4-dicarboxylic acid 4-tert-butyl ester

Di-tert-butyl dicarbonate (3.33 g, 15.3 mmol) was added to a solution of morpholine-3-carboxylic acid (1.7 g, 10.2 mmol), potassium carbonate (7.04 g, 51 mmol) in acetone (5 mL) and water (10 mL) at 0° C. The resulting mixture was stirred at room temperature for 24 h, diluted with water (50 mL) and extracted with diethyl ether (2×50 mL). The aqueous phase was treated with hydrochloric acid (2M aqueous, 100 mL), extracted with dichloromethane (2×50 mL). The combined organic phase was washed with water (50 mL), brine (50 mL), dried (sodium sulfate), filtered and concentrated in-vacuo to isolate the desired product as white solid (1.98 g, 84%). ¹H NMR (CDCl₃), δ (ppm): 4.46 (m, 2H), 3.80 (m, 3H), 3.53 (m, 1H), 3.31 (m, 1H), 1.48 (d, 9H).

### Example 10

### Piperazine-1,2,4-tricarboxylic acid 4-tert-butyl ester 1-(9H-fluoren-9-ylmethyl) ester

A solution of 9-fluorenylmethyl chloroformate (2.72 g, 10.5 mmol) in 1,4-dioxane (19 mL) was added drop-wise to a solution of piperazine-1,3-dicarboxylic acid 1-tert-butyl ester (2.20 g, 9.6 mmol) and N,N-diisopropylethylamine (4.2 mL, 23.9 mmol) in water (9.5 mL) in an ice-bath. After stirring overnight at room temperature, the reaction mixture was diluted with water and extracted with chloroform (4 times). The organic layer was washed with saturated sodium bicarbonate and water and then 1N HCl and water, dried over anhydrous sodium sulfate, filtered, and concentrated to afford piperazine-1,2,4-tricarboxylic acid 4-tert-butyl ester 1-(9H-fluoren-9-ylmethyl) ester (4.3 g).

### Example 11

### 2-Formyl-piperidine-1-carboxylic acid tert-butyl ester

DMSO (7.14 mL, 98 mmol) was added drop-wise to a stirred solution of oxalyl chloride (30 mL, 2M in CH₂Cl₂, 60 mmol) in CH₂Cl₂ (60 mL) at −78° C. After 5 minutes, a solution of 2-hydroxymethyl-piperidine-1-carboxylic acid tert-butyl ester in CH₂Cl₂ (25 mL) was added and the reaction mixture as stirred at −78° C. for 0.5 hours after which Et₃N (25 mL, 181 mmol) was and the mixture allowed to warn slowly to room temperature with stirring. The mixture was then poured into water (100 mL) and the organic layer was separated. The organic extract was then washed with NaHCO₃ (saturated). The aqueous phase was extracted with dichloromethane (3×30 mL). The combined organic phase was washed with water (30 mL) and brine (30 mL), dried (sodium sulfate), filtered and concentrated in vacuo. Chromatography gave the title product as a yellow oil (3.27 g, 73%).

### Example 12

### Morpholine-3,4-dicarboxylic acid 4-tert-butyl ester 3-methyl ester

Iodomethane (0.32 mL, 5.19 mmol) was added to a solution of morpholine-3,4-dicarboxylic acid 4-tert-butyl ester (1 g, 4.32 mmol) and potassium carbonate in DMF (15 mL). The resulting mixture was stirred at room temperature for 4 h, diluted with diethyl ether (100 mL), and successively washed with water (3×100 mL) and brine (100 mL). The organic phase was dried (sodium sulfate), filtered and concentrated in-vacuo to isolate the desired compound as clear oil (0.99 g, 94%). ¹H NMR (CDCl₃), δ (ppm): 4.40 (m, 2H), 3.75 (m, 6H), 3.39 (m, 2H), 1.46 (d, 9H).

### Example 13

### 3-Formyl-morpholine-4-carboxylic acid tert-butyl ester

Diisobutylaluminum hydride (1M in toluene), was added drop-wise to a solution of morpholine-3,4-dicarboxylic acid 4-tert-butyl ester 3-methyl ester (992 mg, 4.05 mmol) in toluene (10 mL) at −78° C., and left stirring at −78° C. for 1 h. The reaction was quenched by slow addition of sodium sulfate decahydrate (0.6 g) with stirring at 80° C. for 40 minutes. The mixture was filtered while hot through a celite pad using ethyl acetate. The filtrate was concentrated in vacuo and chromatography (silica gel, 8% acetone in hexanes) yielded the title product as a white solid (539 mg, 62%). ¹H NMR (CDCl₃), δ (ppm): 9.68 (s, 1H), 4.45 (m, 2H), 3.86 (m, 2H), 3.70 (dd, 1H), 3.51 (m, 1H), 3.23 (m, 1H), 1.48 (m, 9H).

### Example 14

### a) 2-Cyano-piperidine-1-carboxylic acid tert-butyl ester

Piperidine-1,2-dicarboxylic acid-1-tert-butyl ester (12.8 g, 55.6 mmol) and THF (170 mL) were added to a 500 mL round bottom flask equipped with stir bar. The solution was cooled to −20° C. and triethylamine (10.1 mL, 72.3 mmol) was added followed by ethyl chloroformate (5.32 mL, 55.6 mmol). The resulting white precipitate was left stirring at −10° C. for 1 h. Aqueous ammonia (22.6 mL, 1168 mmol) was added to the above reaction mixture and the clear reaction mixture was stirred at room temperature overnight. The reaction mixture was concentrated in vacuo and the isolated residue was dissolved in ethyl acetate (300 mL). The organic phase was successively washed with water (300 mL) and brine (200 mL), dried (sodium sulfate), filtered and concentrated in vacuo to isolate a clear gum. The gum was triturated with hexanes to isolate the carbamate (9.4 g, 74%) as a white solid. ¹H NMR (CDCl₃), δ (ppm): 6.03 (bs, 1H), 5.55 (bs, 1H), 4.77 (bs, 1H), 4.05 (bs, 1H), 2.81 (t, 1H), 2.27 (bs, 1H), 1.47 (m, 14H).

Acetonitrile (220 mL) and DMF (3.82 mL, 49.4 mmol) were added to a 500 mL round bottom flask equipped with stir bar. Cooled the mixture down to −5° C. and to it added oxalyl chloride (24.7 mL, 49.4 mmol, 2 M dichloromethane). The resulting mixture was stirred for 15 min. This was followed by addition of solution of 2-carbamoyl-piperidine-1-carboxylic acid tert-butyl ester (9.4 g, 41.2 mmol) in acetonitrile (50 mL) and pyridine (8.3 mL, 103 mmol). Reaction mixture was left stirring at room temperature overnight. The reaction mixture was concentrated in vacuo and the residue was dissolved in ethyl acetate (300 mL). The organic phase was successively washed with water (300 mL) and brine (200 mL), dried (sodium sulfate), filtered and concentrated in vacuo to isolate the title compound (8.44 g, 97%) as a yellow solid. ¹H NMR (CDCl₃), δ (ppm): 5.23 (bs, 1H), 4.03 (bs, 1H), 2.93 (t, 1H), 1.75 (m, 5H), 1.46 (m, 10H).

### b) tert-Butyl 3-cyanomorpholine-4-carboxylate

Triethylamine (1.808 mL, 12.97 mmol) and ethyl chloroformate (0.909 mL, 9.514 mmol) were added to a cooled (0° C.) solution of morpholine-3,4-dicarboxylic acid 4-tert-butyl ester (2.00 g, 8.65 mmol) in THF (25 mL). The reaction was warmed to room temperature and allowed to stir for 2 h, then cooled to 0° C. and ammonium hydroxide (4 mL) was added. The resulting mixture was warmed to room temperature and stirred for a further 1 h. The solvent was removed in vacuo, and the product was extracted from the aqueous phase with dichloromethane. The combined organics were dried (Na₂SO₄), filtered and concentrated under reduced pressure to yield 3-carbamoyl-morpholine-4-carboxylic acid tert-butyl ester (off-white solid, 1.37 g, 69%). ¹H NMR (300 MHz, CDCl₃) δ=1.51 (s, 9H); 3.19 (m, 1H); 3.52 (m, 2H); 3.88 (m, 2H); 4.50 (d, J=11.4, 1H); 5.81 (s broad, 1H); 6.05 (s broad, 1H).

Oxalyl chloride (3.87 mL of 2M in DCM, 7.73 mmol) was added to a cooled (0° C.) solution of dimethylformamide (0.598 mL, 7.73 mmol) in acetonitrile (15 mL). The solution was stirred for 20 min at 0° C. A solution of 3-carbamoyl-morpholine-4-carboxylic acid tert-butyl ester (1.37 g, 5.95 mmol) in acetonitrile (6 mL) and pyridine (0.481 mL, 5.95 mmol) was added to the first solution. The mixture was allowed to warm to room temperature and stirred for 30 min. The solvent was removed in vacuo, and the resulting residue was dissolved in dichloromethane and washed with water. The aqueous phase was re-extracted with dichloromethane. The combined organics were dried (Na₂SO₄—), filtered and concentrated under reduced pressure to yield the title compound (off white crystals, 1.24 g, 98%). ¹H NMR (300 MHz, CDCl₃) δ=1.51 (s, 9H); 3.26 (m, 1H); 3.55 (td, J=11.8 Hz, 2.7 Hz, 1H); 3.41 (dd, J=11.8 Hz, 3.3 Hz, 1H); 3.83 (m, 1H); 3.98 (d, J=11.4 Hz, 1H); 4.08 (d, J=12 Hz, 1H); 5.32 (m, 1H).

### Example 15

### a) tert-butyl 2-(2H-tetrazol-5-yl)piperidine-1-carboxylate

tert-Butyl 2-cyanopiperidine-1-carboxylate (2.10 g, 10 mmol) was mixed with sodium azide (0.715 g, 11 mmol) and ammonium chloride (0.588 g, 11 mmol) in DMF (7.5 mL) and heated at 100° C. overnight. The reaction mixture was quenched with water and extracted with ethyl acetate. The organic layer was washed with water three times and then with brine, dried and concentrated to give the title compound (white solid, 2.34 g, 92.5%). ¹H NMR (CDCl₃), δ (ppm): 5.7 (m, 1H), 4.02 (m, 1H), 2.93 (m, 1H), 2.35 (m, 1H), 2.07 (m, 1H), 1.74 (m, 3H), 1.49 (m+s, 11H).

The following compound was made in the same manner:

### b) tert-Butyl 3-(2H-tetrazol-5-yl)morpholine-4-carboxylate

tert-Butyl 3-cyanomorpholine-4-carboxylate (2.74 g, 12.9 mmol) was mixed with sodium azide (0.923 g, 14.2 mmol) and ammonium chloride (0.759 g, 14.2 mmol) in DMF (8 mL) and heated at 100° C. for 6 h and left stirring at room temperature overnight. The reaction mixture was quenched with water, acidified to pH 3, and extracted with ethyl acetate. The organic layer was washed with water three times and then with brine, dried and concentrated to give the title compound (white solid, 2.64 g, 80.7%). ¹H NMR (CDCl₃), δ (ppm): 5.5 (br s, 1H), 4.45 (d, 1H), 3.8-3.98 (m, 3H), 3.62 (t, 1H), 3.3 (br s, 1H), 1.46 (s, 9H).

### Example 16

### 2-(Hydroxyimino-methyl)-piperidine-1-carboxylic acid tert-butyl ester

2-Formyl-piperidine-1-carboxylic acid tert-butyl ester (1.0 g, 4.7 mmol) in pyridine (1.3 mL) was added to a solution of hydroxylamine hydrochloride (407 mg, 5.9 mmol) in pyridine (5.0 mL) at 0° C., and the mixture was stirred at room temperature for 12 h. The mixture was diluted with water (50 mL), extracted with dichloromethane (3×25 mL). The combined organic phase was washed with brine (50 mL), dried (sodium sulfate), filtered and concentrated in vacuo to isolate the desired compound as light yellow oil (1.0 g).

### Example 17

### 3-(Hydroxyimino-methyl)-morpholine-4-carboxylic acid tert-butyl ester

A solution of 3-formyl-morpholine-4-carboxylic acid tert-butyl ester (539 mg, 2.50 mmol) in pyridine (1.3 mL) was added to a solution of hydroxylamine hydrochloride (217 mg, 3.13 mmol) in pyridine (2.5 mL) at 0° C. The mixture was warmed to room temperature and stirred for 12 h, diluted with water (50 mL), and extracted with dichloromethane (3×25 mL). The combined organic phase was washed with brine (50 mL), dried (sodium sulfate), filtered and concentrated, in-vacuo to isolate the desired compound as light yellow oil (578 mg).

### Example 18

### 2-[5-(3-Chloro-phenyl)-isoxazol-3-yl]-piperidine-1-carboxylic acid tert-butyl ester

N-chlorosuccinimide (643 mg, 4.82) in DMF (6 mL) was added to 2-(hydroxyimino-methyl)-piperidine-1-carboxylic acid tert-butyl ester (1.0 g, 4.38 mmol) in dimethylformamide (10 mL) at 40° C. The mixture was stirred at 40° C. for 1.5 h, cooled to room temperature, diluted with diethyl ether (75 mL), and sequentially washed with water (3×100 mL) and brine (100 mL). The organic phase was dried (sodium sulfate), filtered and concentrated in vacuo, to give the intermediate as a yellow oil.

The intermediate in dichloromethane (5 mL) was added to 3-chloro-1-ethynylbenzene (1.24 mL, 10 mmol) and triethylamine (1.05 mL, 7.54 mmol) and dichloromethane (5 mL) at 0° C. and the mixture was stirred at room temperature for 12 h, and concentrated in vacuo. The residue was dissolved in ethyl acetate (75 mL), and sequentially washed with water (3×50 mL) and brine (50 mL). The organic phase was dried (sodium sulfate), filtered and concentrated in vacuo. Chromatography (silica gel, 2% ethyl acetate in dichloromethane) gave the title compound as a yellow solid (236 mg). ¹H NMR (CDCl₃), δ (ppm): 7.75 (dd, 1H), 7.64 (m, 1H), 7.40 (m, 2H), 6.37 (s, 1H), 5.48 (br, 1H), 4.08 (m, 1H), 2.83 (m, 1H), 2.35 (m, 1H), 2.00-1.53 (m, 5H), 1.52 (s, 9H).

### Example 19

### 3-[5-(3-chloro-phenyl)-isoxazol-3-yl]-morpholine-4-carboxylic acid tert-butyl ester

A solution of N-chlorosuccinimide in dimethylformamide (6 mL) was added to a solution of 3-(hydroxyimino-methyl)-morpholine-4-carboxylic acid tert-butyl ester (578 mg, 2.51 mmol) in dimethylformamide (10 mL) at 40° C., and the mixture was stirred at 40° C. for 1.5 h. The reaction mixture was cooled to room temperature, diluted with diethyl ether (75 mL), sequentially washed with water (3×100 mL) and brine (100 mL). The organic phase was dried (sodium sulfate), filtered and concentrated, in-vacuo, to isolate the intermediate as clear oil.

The intermediate in dichloromethane (5 mL) was added to a solution of 3-chloro-1-ethynylbenzene (1.24 mL, 10 mmol), triethylamine (1.05 mL, 7.54 mmol) in dichloromethane (5 mL) at 0° C. and the mixture was stirred at room temperature for 12 h. The reaction mixture was concentrated in vacuo, dissolved in ethyl acetate (75 mL), and sequentially washed with water (3×50 mL) and brine (50 mL). The organic phase was dried (sodium sulfate), filtered and concentrated, in-vacuo. Chromatography (silica gel, 2% ethyl acetate in dichloromethane) yielded the title compound as a yellow solid (236 mg). ¹H NMR (CDCl₃), δ (ppm): 7.76 (bs, 1H), 7.67 (m, 1H), 7.43 (m, 2H), 6.51 (s, 1H), 5.24 (m, 1H), 4.39 (d, 1H), 3.88 (m, 3H), 3.60 (dt, 1H), 3.24 (m, 1H), 1.52 (s, 9H).

### Example 20

### 3-[3-(3-Chloro-phenyl)-[1,2,4]oxadiazol-5-yl]-morpholine-4-carboxylic acid tert-butyl ester

Isobutyl chloroformate (0.42 mL, 3.24 mmol) was added to a solution of morpholine-3,4-dicarboxylic acid 4-tert-butyl ester (500 mg, 2.16 mmol) and triethylamine (0.805 mL, 5.79 mmol) in THF (15 mL) at 0° C. The mixture was warmed to room temperature for 2 hours. 3-Chloro-N-hydroxy-benzamidine was added (368 mg, 2.16 mmol) and the mixture was stirred overnight at room temperature, then cooled and diluted with ethyl acetate (350 mL). The organic layer was washed with water (2×30 mL) and brine (30 mL), dried over anhydrous sodium sulfate, filtered and concentrated in vacuo. Chromatography (silica gel, 30-40% ethyl acetate in hexanes) yielded the ester (755 mg, 91%). ¹H NMR (CDCl₃), δ (ppm): 7.73 (s, 1H), 7.60 (d, 1H), 7.47 (d, 1H), 7.38 (dd, 1H), 5.25 (d, 2H), 4.4-4.8 (m, 2H), 4.1-3.2 (m, 5H), 1.50 (s, 9H).

A solution of the ester in DMF was heated at 127° C. for 2 hours. The product was extracted into ethyl acetate (100 mL) and the organic layer was washed with water (3×20 mL) and brine (20 mL), dried over anhydrous sodium sulfate, filtered and concentrated in vacuo. The title compound (783 mg) was obtained in quantitative yield. ¹H NMR (CDCl₃): 8.09 (s, 1H), 7.98 (d, 1H), 7.46 (m, 2H), 4.50 (s, 1H), 4.2-3.2 (m, 6H), 1.49 (s, 9H).

### Example 21

### 3-[3-(3-Chloro-phenyl)-[1,2,4]oxadiazol-5-yl]-piperazine-1-carboxylic acid tert-butyl ester

Piperazine-1,2,4-tricarboxylic acid 4-tert-butyl ester 1-(9H-fluoren-9-ylmethyl) ester (4.3 g, 9.6 mmol), 3-chloro-N-hydroxy-benzamidine (1.8 g, 10.5 mmol), HOBt (1.4 g, 10.5 mmol) and EDCI (2.0 g, 10.5 mmol) in DMF (25 mL) were stirred at room temperature overnight. The reaction mixture was diluted with ethyl acetate, washed with water (3 times), saturated sodium bicarbonate and brine, dried over anhydrous sodium sulfate, filtered and concentrated. The residue was dissolved in DMF (20 mL) and then heated at 135° C. for 2 hours. After cooling, the reaction mixture was diluted with ethyl acetate, washed with water (3 times) and brine, dried over anhydrous sodium sulfate, filtered and concentrated. Chromatography (silica gel, hexanes to 1:1 hexanes:dichloromethane to 1:3:4 ethyl acetate:hexanes:dichloromethane to 3:1:4 ethyl acetate:hexanes dichloromethane) afforded the title compound (1.35 g, 39%). ¹H NMR (CDCl₃) δ (ppm): 8.12 (m, 1H), 8.00 (m, 1H), 7.47 (m, 2H), 4.21 (m, 2H), 3.81 (m, 1H), 3.25 (m, 2H), 2.81 (m, 2H), 2.38 (bs, 1H), 1.50 (bs, 9H).

### Example 22

### a) tert-Butyl 2-[2-(3-chlorophenyl)-2H-tetrazol-5-yl]piperidine-1-carboxylate

A mixture of tert-butyl 2-(2H-tetrazol-5-yl)piperidine-1-carboxylate (253 mg, 1 mmol), sodium t-butoxide (96 mg, 1 mmol), rac-BINAP (24.9 mg, 0.04 mmol), Pd₂(dba)₃ (10.4 mg, 0.01 mmol), copper(II) 2-phenylcyclopropanecarboxyate (7.72 mg, 0.02 mmol) and bis-(3-chloro-phenyl)-iodonium tetrafluoroborate (436.8 mg, 1 mmol) was refluxed in t-butanol (20 mL) under argon for two hours. After the solvent was removed in vacuo, chromatography (5% ethyl acetate in hexanes) gave the title compound (pale-yellow sticky oil, 237.8 mg, 65.3%). ¹H NMR (CDCl₃), δ (ppm): 8.14 (d, 1H), 8.03 (dm, 1H), 7.46 (m, 2H), 5.75 (br s, 1H), 4.1 (m, 1H), 3.05 (m, 1H), 2.43 (d, 1H), 1.99 (tm, 1H), 1.7 (t, 2H), 1.53 (m+s, 11H).

The following compound was made in the same manner:

### b) tert-Butyl 3-[2-(3-chlorophenyl)-2H-tetrazol-5-yl]morpholine-4-carboxylate

A mixture of tert-Butyl 3-(2H-tetrazol-5-yl)morpholine-4-carboxylate (701 mg, 2.74 mmol), sodium t-butoxide (264 mg, 2.74 mmol), rac-BINAP (68.5 mg, 0.11 mmol), Pd₂(dba)₃ (28.4 mg, 0.0274 mmol), copper(II) (1R,2R)-2-phenylcyclopropanecarboxyate (21.2 mg, 0.059 mmol) and bis-(3-chloro-phenyl)-iodonium tetrafluoroborate (1200 mg, 2.74 mmol) was refluxed in t-butanol (40 mL) under argon for two hours. After the solvent was removed in vacuo, chromatography (5-20% ethyl acetate in hexanes) gave the title compound (colorless sticky oil, 840 mg, 83.7%). ¹H NMR (CDCl₃), δ (ppm): 8.14 (s, 1H), 8.03 (dm, 1H), 7.48 (m, 2H), 5.40 (br s, 1H), 4.56 (d, 1H), 3.94 (dd, 1H), 3.90 (m, 2H), 3.62 (td, 1H), 3.47 (br s, 1H).

### Example 23

### 2-[5-(3-Chloro-phenyl)-isoxazol-3-yl]-piperidine

Trifluoroacetic acid (5 mL) was added to 2-[5-(3-chloro-phenyl)-isoxazol-3-yl]-piperidine-1-carboxylic acid tert-butyl ester (500 mg, 1.38 mmol) in dichloromethane (5 mL) and the mixture was stirred at room temperature for 1 h, concentrated to dryness, and the residue was dissolved in sodium hydroxide (1N aqueous, 30 mL). The aqueous phase was extracted with dichloromethane (3×30 mL). The combined organic phase was washed with water (30 mL) and brine (30 mL), dried (sodium sulfate), filtered and concentrated in vacuo to give the title compound as light yellow oil (292 mg, 81%). ¹H NMR (CDCl₃), δ (ppm): 7.75 (dd, 1H), 7.65 (m, 1H), 7.41 (m, 2H), 6.60 (s, 1H), 3.94 (dd, 1H), 3.17 (m, 1H), 2.83 (m, 1H), 2.35 (m, 1H), 2.00-1.53 (m, 6H).

### Example 24

### 3-[5-(3-Chloro-phenyl)-isoxazol-3-yl]-morpholine

Trifluoroacetic acid (2 mL) was added to 3-[5-(3-chloro-phenyl)-isoxazol-3-yl]-morpholine-4-carboxylic acid tert-butyl ester (236 mg, 0.65 mmol) in dichloromethane (2 mL). The mixture was stirred at room temperature for 1 h, concentrated to dryness, and the residue was dissolved in sodium hydroxide (1N aqueous, 30 mL). The aqueous phase was extracted with dichloromethane (3×30 mL). The combined organic phase was washed with water (30 mL) and brine (30 mL), dried (sodium sulfate), filtered and concentrated in vacuo to yield the title compound as light yellow oil (171 mg, 99%). ¹H NMR (CDCl₃), δ (ppm): 7.72 (s, 1H), 7.62 (m, 1H), 7.37 (m, 2H), 6.59 (s, 1H), 4.18 (dd, 1H), 4.00 (dd, 1H), 3.87 (dt, 1H), 3.62 (m, 2H), 3.03 (m, 2H), 2.10 (bs, 1H).

### Example 25

### 3-[3-(3-Chloro-phenyl)-[1,2,4]oxadiazol-5-yl]-morpholine

A solution of the 3-[3-(3-Chloro-phenyl)-[1,2,4]oxadiazol-5-yl]-morpholine-4-carboxylic acid tert-butyl ester (783 mg, 2.19 mmol) was dissolved in a minimum amount of dichloromethane and then cooled to 0° C. in an ice bath. A 1:1 solution of trifluoroacetic acid: dichloromethane (10 mL) was added and the mixture stirred at 0° C. for 15 minutes, and the mixture was warmed to RT for 45 minutes. Ice cold water (20 mL) was added and the mixture was neutralized with saturated sodium bicarbonate. The product was extracted into dichloromethane (2×25 mL) and washed with brine (2×25 mL), dried over anhydrous sodium sulfate, filtered and concentrated in vacuo. Chromatography (silica gel) yielded the title compound (429 mg, 74%). ¹H NMR (CDCl₃), δ (ppm): 8.11 (s, 1H), 8.00 (d, 1H), 7.47 (m, 2H), 3.6-4.4 (m, 6H), 3.0-3.3 (m, 2H).

### Example 26

### a) 2-[2-(3-Chloro-phenyl)-2H-tetrazol-5-yl]-piperidine

tert-Butyl 2-[2-(3-chlorophenyl)-2H-tetrazol-5-yl]piperidine-1-carboxylate (237 mg, 0.651 mmol) was mixed with trifluoroacetic acid (0.85 mL) and dichloromethane (0.85 mL) at 0° C. 0.5 hour. The mixture was poured into saturated sodium carbonate and extracted with dichloromethane. Chromatography (20-100% ethyl acetate in hexanes) gave 2-[2-(3-chlorophenyl)-2H-tetrazol-5-yl]piperidine (white solid, 113 mg, 65.8%). ¹H NMR (CDCl₃), δ (ppm): 8.16 (s, 1H), 8.03 (dm, 1H), 7.46 (m, 2H), 4.17 (dm, 1H), 3.21 (dm, 1H), 2.84 (tm, 1H), 2.18 (dm, 1H), 2.15 (m, 1H), 1.94 (m, 1H), 1.8 (m, 1H), 1.68 (m, 1H), 1.59 (m, 2H).

The following compound was made in the same manner:

### b) 3-[2-(3-chlorophenyl)-2H-tetrazol-5-yl]morpholine

tert-Butyl 3-[2-(3-chlorophenyl)-2H-tetrazol-5-yl]morpholine-4-carboxylate (840 mg, 2.296 mmol) was mixed with trifluoroacetic acid (6 mL) and dichloromethane (6 mL) at 0° C. 1.5 hour. The mixture was poured into saturated sodium carbonate and extracted with dichloromethane, dried and concentrated to yield the title compound (pale yellow sticky oil, 550 mg, 90%). ¹H NMR (CDCl₃), δ (ppm): 8.18 (s, 1H), 8.06 (dm, 1H), 7.52 (m, 2H), 4.45 (dd, 1H), 4.24 (dd, 1H), 3.92 (dt, 1H), 3.87 (dd, 1H), 3.72 (ddd, 1H), 3.14 (m, 2H), 2.11 (br s, 1H).

### Example 27

### 2-[5-(3-Chloro-phenyl)-isoxazol-3-yl]-piperidine-1-carbothioic acid methylamide

Methyl isothiocyanate (63 mg, 0.86 mmol) was added to 2-[5-(3-Chloro-phenyl)-isoxazol-3-yl]-piperidine (150 mg, 0.57 mmol) in CH₂Cl₂ (4 mL) and the resulting mixture was stirred at room temperature for 12 h. The mixture was concentrated in vacuo and the isolated residue was triturated with 50% diethyl ether in hexanes to isolate the desired compound as off-white solid (quantitative).

### Example 28

### 3-[5-(3-Chloro-phenyl)-isoxazol-3-yl]-morpholine-4-carbothioic acid methylamide

Methyl isothiocyanate (46.2 mg, 0.63 mmol) was added to 3-[5-(3-chloro-phenyl)-isoxazol-3-yl]-morpholine (145 mg, 0.55 mmol) in CHCl₃ (4 mL) and the resulting mixture was stirred at room temperature for 12 h. The mixture was concentrated in vacuo and the isolated residue was triturated with 50% diethyl ether in hexanes to isolate the title compound as off-white solid (181 mg, 97%). ¹H NMR (CDCl₃), δ (ppm): 7.78 (m, 1H), 7.67 (m, 1H), 7.45 (m, 2H), 6.75 (s, 1H), 6.28 (m, 1H), 5.80 (m, 1H), 4.57 (d, 1H), 4.29 (d, 1H), 4.09 (dd, 1H), 3.99 (dd, 1H), 3.75 (dt, 1H), 3.45 (dt, 1H), 3.23 (d, 3H).

### Example 29

### 3-[3-(3-Chloro-phenyl)-[1,2,4]oxadiazol-5-yl]-morpholine-4-carbothioic acid methylamide

Methyl isothiocyanate (161 mg, 2.2 mmol) and Et₃N (0.61 mg, 4.4 mmol) were added to a solution of 3-[3-(3-Chloro-phenyl)-[1,2,4]oxadiazol-5-yl]-morpholine (294 mg, 1.1 mmol) in CH₂Cl₂ (4 mL) and the mixture was stirred at room temperature for 12 h, and concentrated in vacuo. Chromatography gave the title compound as viscous oil (313 mg, 84%). ¹H NMR (CDCl₃), δ (ppm): 8.06 (d, 1H), 7.96 (dd, 1H), 7.48 (dd, 1H), 7.45 (t, 1H), 6.88 (dd, 1H), 6.01 (br, m, 1H), 4.57 (d, 1H), 3.99 (m, 2H), 3.80 (m, 2H), 3.67 (ddd, 1H), 3.26 (d,3H).

### Example 30

### 3-[3-(3-Chloro-phenyl)-[1,2,4]oxadiazol-5-yl]-4-methylthiocarbamoyl-piperazine-1-carboxylic acid tert-butyl ester

Methyl isothiocyanate (256 mg, 3.50 mmol) was added to a solution of 3-[3-(3-chloro-phenyl)-[1,2,4]oxadiazol-5-yl]-piperazine-1-carboxylic acid tert-butyl ester (1.11 g, 3.04 mmol) in chloroform (17 mL) at room temperature. After stirring overnight, the mixture was concentrated and chromatography (silica gel, 1:3:4 ethyl acetate:hexanes:dichloromethane to 1.5:2.5:4 ethyl acetate:hexanes:dichloromethane) afforded the title compound (796 mg, 60%). ¹H NMR (CDCl₃) δ (ppm): 8.05 (m, 1H), 7.95 (m, 1H), 7.45 (m, 2H), 6.01 (m, 1H), 4.68 (m, 1H), 4.22 (m, 1H), 3.80 (m, 2H), 3.51 (m, 1H), 3.25 (m, 3H), 3.07 (m, 1H), 1.30 (bs, 9H).

### Example 31

### 2-[2-(3-Chloro-phenyl)-2H-tetrazol-5-yl]-piperidine-1-carbothioic acid methylamide

2-({2-[2-(3-chlorophenyl)-2H-tetrazol-5-yl]piperidin-1-yl}methyl)pyridine (600 mg, 2.38 mmoles) was mixed with methyl isothiocyante (250 mg, 3.41 mmol) in chloroform (10 mL) at room temperature overnight. The reaction mixture was concentrated and triturated with ether to give the title compound as a white solid (676 mg, 88%). ¹H NMR (CDCl₃), δ (ppm): 8.13 (s, 1H), 8.03 (m, 1H), 7.51 (m, 2H), 6.93 (w, 1H), 6.06 (w, 1H), 4.24 (m 1H), 3.34 (m, 1H), 3.23 (d, 3H), 2.46 (m, 1H), 2.11 (m, 1H), 1.60-1.95 (m, 4H).

### Example 32

### 2-[5-(3-Chloro-phenyl)-isoxazol-3-yl]-N-methyl-piperidine-1-carboximidothioic acid methyl ester

Iodomethane (50 μl, 0.80 mmol) was added to 2-[5-(3-Chloro-phenyl)-isoxazol-3-yl]-piperidine-1-carbothioic acid methylamide (181 mg, 0.54 mmol) in methanol (4 mL) and the resulting mixture was stirred at 75° C. for 3 h. The mixture was cooled to room temperature, diluted with saturated sodium bicarbonate (aqueous, 30 mL), extracted with dichloromethane (3×20 mL). The combined organic phase was washed with brine (30 mL), dried (sodium sulfate), filtered and concentrated in vacuo to yield the title compound as yellow oil (0.19 g, 100%). ¹H NMR (CDCl₃), δ (ppm): 7.73 (dd, 1H), 7.64 (m, 1H), 7.38 (m, 2H), 6.60 (s, 1H), 5.37 (m, 1H), 4.25 (m, 1H), 3.95 (m, 2H), 3.67 (m, 2H), 3.32 (m, 1H), 3.25 (s, 3H), 2.36 (s, 3H).

### Example 33

### 3-[5-(3-Chloro-phenyl)-isoxazol-3-yl]-N-methyl-morpholine-4-carboximidothioic acid methyl ester

Iodomethane (50 μl, 0.80 mmol) was added to 3-[5-(3-chloro-phenyl)-isoxazol-3-yl]-morpholine-4-carbothioic acid methylamide (181 mg, 0.54 mmol) in methanol (4 mL) and the resulting mixture was stirred at 75° C. for 3 h. The mixture was cooled to room temperature, diluted with saturated sodium bicarbonate (aqueous, 30 mL), extracted with dichloromethane (3×20 mL). The combined organic phase was washed with brine (30 mL), dried (sodium sulfate), filtered and concentrated in vacuo to yield the title compound as yellow oil (0.19 g, 100%). ¹H NMR (CDCl₃), δ (ppm): 7.73 (dd, 1H), 7.64 (m, 1H), 7.38 (m, 2H), 6.60 (s, 1H), 5.37 (m, 1H), 4.25 (m, 1H), 3.95 (m, 2H), 3.67 (m, 2H), 3.32 (m, 1H), 3.25 (s, 3H), 2.36 (s, 3H).

### Example 34

### 3-[3-(3-Chloro-phenyl)-[1,2,4]oxadiazol-5-yl]-methylmorpholine-4-carboximidothioic acid methyl ester

Iodomethane (212 mg, 1.5 mmol) was added to a solution of 3-[3-(3-chloro-phenyl)[1,2,4]oxadiazol-5-yl]-morpholine-4-carbothioic acid methylamide (313 mg, 0.92 mmol) in methanol (10 mL) and the mixture was stirred at 75° C. for 3 h. The mixture was cooled to room temperature, diluted with saturated sodium bicarbonate (aqueous, 30 mL), extracted with dichloromethane (3×20 mL). The combined organic phase was washed with brine (30 mL), dried (sodium sulfate), filtered and concentrated in vacuo to yield the title compound as a white solid (248 mg, 76%). ¹H NMR (CDCl₃), δ (ppm) 8.08 (d, 1H), 7.95 (dd, 1H), 7.47 (dd, 1H), 7.43 (t, 1H), 5.47 (dd, 1H), 4.36 (d, 1H), 3.40-4.00 (m, 5H), 3.21 (s, 3H), 2.36 (s,3H).

### Example 35

### 3-[3-(3-Chloro-phenyl)-[1,2,4]oxadiazol-5-yl]-4-(methylimino-methylsulfanyl-methyl)-piperazine-1-carboxylic acid tert-butyl ester

3-[3-(3-Chloro-phenyl)-[1,2,4]oxadiazol-5-yl]-4-methylthiocarbamoyl-piperazine-1-carboxylic acid tert-butyl ester (796 mg, 1.82 mmol) and iodomethane (0.170 mL, 2.73 mmol) in methanol (11 mL) were heated at 75° C. in a sealed vial for 2 hours. After cooling, the mixture was concentrated and then the residue was dissolved with dichloromethane. The organic layer was washed with saturated sodium bicarbonate, dried over anhydrous sodium sulfate, filtered, and concentrated. Chromatography (silica gel, 25% ethyl acetate in hexanes) afforded the title compound (632 mg, 77%). ¹H NMR (CDCl₃) δ (ppm): 8.08 (m, 1H), 7.97 (m, 1H), 7.44 (m, 2H), 5.51 (m, 1H), 4.49 (m, 1H), 4.01 (m, 2H), 3.49 (m, 2H), 3.20 (s, 3H), 3.15 (m, 1H), 2.37 (s, 3H), 1.38 (bs, 9H).

### Example 36

### 2-[2-(3-Chloro-phenyl)-2H-tetrazol-5-yl]-N-methyl-piperidine-1-carboximidothioic acid methyl ester

2-[2-(3-Chloro-phenyl)-2H-tetrazol-5-yl]-piperidine-1-carbothioic acid methylamide (676 mg, 2.0 mmol) was mixed with iodomethane (0.4 mL) in methanol (15 mL) in a sealed vial at 80° C. for 2 hours. The reaction mixture was concentrated by rotavapor. The residue was basified with saturated sodium bicarbonate and extracted with dichloromethane. The organic layer was dried with MgSO₄ to give the title compound as a sticky pale-yellow oil (700 mg, 100%). ¹H NMR (CDCl₃), δ (ppm): 8.15 (s, 1H), 8.04 (d, 1H), 7.48 (m, 2H), 5.75 (m, 1H), 3.22 (m, 1H), 3.22 (m,s, 4H), 2.04 (s,m, 4H), 2.10 (m, 1H), 1.69 (m, 4H).

### Example 37

### a) 4-(5-{2-[5-(3-Chloro-phenyl)-isoxazol-3-yl]-piperidin-1-yl}-4-methyl-4H [1,2,4]triazol-3-yl)-pyridine

Isonicotinic acid hydrazide (42.3 mg, 0.31 mmol) was added to 2-[5-(3-Chloro-phenyl)-isoxazol-3-yl]N-methy-l-piperidine-1-carboximidothioic acid methyl ester (90 mg, 0.26 mmol) in ethanol (1.5 mL). The mixture was stirred at 75° C. for 12 h, and then diluted with dichloromethane (8 mL). The organic phase was sequentially washed with water (4×10 mL) and brine (10 mL), dried (sodium sulfate), filtered and concentrated in vacuo. Chromatography (silica gel, 10% methanol in ethyl acetate) gave a yellow oil that was triturated with 30% hexanes in diethyl ether to yield the title compound as an off-white solid (50 mg). ¹H NMR (CDCl₃), δ (ppm): 8.72 (d, 2H), 7.69 (s, 1H), 7.59 (m, 3H), 7.36 (m, 2H), 6.54 (s, 1H), 4.79 (dd, 1H), 3.64 (s, 3H), 3.28 (m, 2H), 2.20 (m, 2H), 1.90-1.73 (m, 4H).

The following compounds were prepared in a similar manner:

b) 3-[5-(3-Chlorophenyl)-[1,2,4]oxadioazol-3-yl]-4-(5-pyridin-4-yl-4H-[1,2,4]triazol-3-yl)-morpholine; yield 40.2 mg, 24%, yellow powder; ¹H NMR CDCl₃ (300 MHz): 3.37 (m, 1H); 3.59 (m, 1H); 3.75 (s, 3H); 3.97 (m, 1H); 4.08 (m, 2H); 4.32 (dd, J=11.7 Hz, 3.3 Hz, 1H); 5.00 (m, 1H); 7.45 (t, J=8 Hz, 7.56 (d, J=8 Hz, 1H); 7.62 (d, J=4.8 Hz, 2H); 7.94 (d, J=7.8 Hz, 1H); 8.04 (m, 1H); 8.75 (br. s, 2H)

Enantiomers were separated using a Chiralpak AD 4.6×250 mm column, eluting with iPrOH/0.05% Et₂NH at a flowrate of 1 mL/min, to yield 12.5 mg of enantiomer 1, Rt 7.39 min. and 12.7 mg of enantiomer 2, Rt 12.57 min.

c) 3-[5-(3-Chlorophenyl)isoxazol-3-yl]-4-(4-cyclopropyl-5-pyridin-3-yl-4H-1,2,4-triazol-3-yl)morpholine; yield 63.5 mg, 27%, off white solid; ¹H NMR CDCl₃ (300 MHz): 9.07 (s, 1H) 8.71 (d of d, 1H), 8.16 (d of t, 1H), 7.75 (d, 1H), 7.64 (m, 1H), 7.41 (m, 3H), 6.83 (s, 1H), 5.18 (t, 1H), 4.25 (d, 2H), 4.12 (m, 1H), 3.99 (m, 1H), 3.72 (m, 1H), 3.49 (m, 1H), 3.38 (m, 1H), 1.30 (m, 1H), 1.14 (m, 2H), 0.60 (m, 1H).

Enantiomers were separated using a Chiralpak AD 4.6×250 mm column, eluting with iPrOH at a flowrate of 1 mL/min, to yield enantiomer 1 as an off-white solid, 14.4 mg, Rt 5.9 min. and enantiomer 2 as an off-white solid, 16.7 mg, Rt 23.7 min.

d) 3-[5-(3-chlorophenyl)isoxazol-3-yl]-4-(4-cyclopropyl-5-pyridin-4-yl-4H-1,2,4-triazol-3-yl)morpholine; yield 103.4 mg, 43%, white solid; ¹H NMR CDCl₃ (300 MHz): 8.75 (d, 2H), 7.76 (m, 3H), 7.64 (m, 1H), 7.41 (m, 2H), 6.83 (s, 1H), 5.19(t, 1H), 4.25(d, 2H), 4.13 (m, 1H), 3.99 (t of d, 1H), 3.73(t of d, 1H), 3.50 (m, 1H), 3.41(m, 1H), 1.28 (m, 1H), 1.15 (m, 2H), 0.62 (m, 1H).

e) 3-[5-(3-chlorophenyl)isoxazol-3-yl]-4-(4-methyl-5-pyridin-3-yl-4H-1,2,4-triazol-3-yl)morpholine; yield 85.0 mg, 35%, white solid; ¹H NMR CDCl₃ (300 MHz): 8.90 (d, 1H), 8.72 (m, 1H), 8.05 (d oft, 1H), 7.73 (m, 1H), 7.61 (m, 1H), 7.41 (m, 3H), 6.67 (s, 1H), 4.82 (m, 1H), 4.25 (d of d, 1H), 4.08 (m, 3H), 3.67 (s, 3H), 3.48 (m, 1H), 3.40 (m, 1H).

f) 3-[5-(3-Chloro-phenyl)-isoxazol-3-yl]-4-[5-(6-methoxy-pyridin-3-yl)-4-methyl-4H-[1,2,4]triazol-3-yl]-morpholine; yield 73.2 mg, 29%, off white solid; ¹H NMR CDCl₃ (300 MHz): 8.40 (d, 1H), 7.88 (d of d, 1H), 7.69 (s, 1H), 7.59 (m, 1H), 7.38 (m, 2H), 6.84(d, 1H), 6.65(s, 1H), 4.79 (m, 1H), 4.20 (d of d, 1H), 4.04 (m, 3H), 3.98 (s, 3H), 3.61(s, 3H), 3.44 (m, 1H), 3.36 (m, 1H).

g) 3-[3-(3-chlorophenyl)-1,2,4-oxadiazol-5-yl]-4-[5-(2-methoxypyridin-4-yl)-4-methyl-4H-1,2,4-triazol-3-yl]morpholine; yield 26.6 mg, 5.8%, yellow oil; ¹H NMR CDCl₃ (300 MHz): 8.31 (d, 1H), 8.04 (t, 1H), 7.95 (dt, 1H), 7.44 (m, 2H), 7.24 (d, 1H), 7.02 (s, 1H), 5.14 (dd, 1H), 4.38 (dd, 1H), 4.19 (dd, 1H), 4.05 (m, 2H), 4.02(s, 3H), 3.73 (s, 3H), 3.7 (m, 1H), 3.34 (m, 1H).

h) 3-[3-(3-chlorophenyl)-1,2,4-oxadiazol-5-yl]-4-[5-(2-methylpyridin-4-yl)-4-methyl-4H-1,2,4-triazol-3-yl]morpholine; yield 42.3 mg, 9.6%, yellow oil; ¹H NMR CDCl₃ (300 MHz): 8.64 (br, 1H), 8.02 (t, 1H), 7.94 (dt, 1H), 7.44 (m, 4H), 5.14 (dd, 1H), 4.38 (dd, 1H), 4.19 (dd, 1H), 4.03 (m, 2H), 3.74 (s, 3H), 3.7 (m, 1H), 3.38 (m, 1H), 2.66 (s, 3H).

i) 3-[3-(3-chlorophenyl)-1,2,4-oxadiazol-5-yl]-4-[5-(5-fluoropyridin-3-yl)-4-methyl-4H-1,2,4-triazol-3-yl]morpholine; yield 285 mg, 63.9%, yellow oil; ¹H NMR CDCl₃ (300 MHz): 8.72 (s, 1H), 8.59 (d, 1H), 8.03 (t, 1H), 7.94 (dt, 1H), 7.82 (dq, 1H), 7.45 (m, 2H), 5.14 (dd, 1H), 4.38 (dd, 1H), 4.19 (dd, 1H), 4.05 (m, 2H), 3.75 (s, 3H), 3.7 (m, 1H), 3.38 (m, 1H).

j) 3-[5-(3-chlorophenyl)isoxazol-3-yl]-4-[5-(5-fluoropyridin-3-yl)-4-methyl-4H-1,2,4-triazol-3-yl]morpholine; yield 40 mg, 38%, off-white solid; ¹H NMR CDCl₃ (300 MHz): 8.73 (s, 1H), 8.59 (d, 1H), 7.83 (m, 1H), 7.73 (m, 1H), 7.62 (m, 1H), 7.41 (m, 2H), 6.68 (s, 1H), 4.83 (m, 1H), 4.25 (m, 1H), 4.08 (m, 3H), 3.71 (s, 3H), 3.45 (m, 2H)

k) 3-[3-(3-chlorophenyl)-1,2,4-oxadiazol-5-yl]-4-(4-methyl-5-pyridin-2-yl-4H-1,2,4-triazol-3-yl)morpholine; yield 68 mg, 14.3%, yellow oil; 90% pure by NMR; ¹H NMR CDCl₃ (300 MHz): 8.64 (d, 1H), 8.22 (d, 1H), 8.01 (s, 1H), 7.93 (d, 1H), 7.78 (td, 1H), 7.28 (m, 3H), 5.14 (dd, 1H), 4.38 (dd, 1H), 4.19 (dd, 1H), 4.03 (m, 2H), 4.02(s, 3H), 3.66 (m, 1H), 3.34 (m, 1H).

l) 4-[5-(5-fluoropyridin-3-yl)-4-methyl-4H-1,2,4-triazol-3-yl]-3-[3-(3-iodophenyl)-1,2,4-oxadiazol-5-yl]morpholine; yield 103 mg, 36.2%, clear oil; ¹H NMR CDCl₃ (300 MHz): 8.74 (s, 1H), 8.61 (d, 1H), 8.38 (t, 1H), 8.02 (dt, 1H), 7.84 (dq, 2H), 7.21 (t, 1H), 5.14 (dd, 1H), 4.38 (dd, 1H), 4.19 (dd, 1H), 4.03 (m, 2H), 3.75(s, 3H), 3.70 (m, 1H), 3.38 (m, 1H)

m) 3-[3-(3-iodophenyl)-1,2,4-oxadiazol-5-yl]-4-(4-methyl-5-pyridin-4-yl-4H-1,2,4-triazol-3-yl)morpholine; yield 99.6 mg, 37.3%, clear oil; ¹H NMR CDCl₃ (300 MHz): 8.78 (dd, 2H), 8.38 (t, 1H), 8.02 (dt, 1H), 7.84 (dt, 1H), 7.63 (dd, 2H), 7.21 (t, 1H), 5.14 (dd, 1H), 4.38 (dd, 1H), 4.18 (m, 1H), 4.03 (m, 2H), 3.76 (s, 3H), 3.71 (m, 1H), 3.37 (m, 1H).

n) 3-[5-(3-chlorophenyl)isoxazol-3-yl]-4-[5-(2-methylpyridin-4-yl)-4-methyl-4H-1,2,4-triazol-3-yl]morpholine; yield 5.6 mg, 5%, yellow oil; ¹H NMR CDCl₃ (300 MHz): 8.64 (d, 1H), 7.72 (m, 1H), 7.5 (m, 1H), 7.41 (m, 1H), 7.38 (m, 3H), 6.66 (s, 1H), 4.81 (m, 1H), 4.24 (m, 1H), 4.09 (m, 3H), 3.68 (s, 3H), 3.52 (m, 2H), 2.63 (s, 3H)

### Example 38

### 3-[5-(3-Chloro-phenyl)-isoxazol-3-yl]-4-(4-methyl-5-pyridin-4-yl-4H-[1,2,4]triazol-3-yl)-morpholine

Isonicotinic acid hydrazide (56.1 mg, 0.41 mmol) was added to 3-[5-(3-chloro-phenyl)-isoxazol-3-yl]-N-methyl-morpholine-4-carboximidothioic acid methyl ester (96 mg, 0.27 mmol) in ethanol. The resulting mixture was left stirring at 75° C. for 12 h, and then diluted with dichloromethane (8 mL). The organic phase was sequentially washed with water (4×10 mL) and brine (10 mL), dried (sodium sulfate), filtered and concentrated in vacuo. Chromatography (silica gel, 10% methanol in ethyl acetate) gave a yellow oil that was triturated with 30% hexanes in diethyl ether to yield the title compound as an off-white solid (46 mg). ¹H NMR (CDCl₃), δ (ppm): 8.76 (d, 2H), 7.72 (dd, 1H), 7.62 (m, 3H), 7.42 (m, 2H), 6.67 (s, 1H), 4.82 (dd, 1H), 4.25 (dd, 1H), 4.07 (m, 3H), 3.71 (s, 3H), 3.45 (m, 2H).

Enantiomers were separated using a Chiralpak AD 4.6×250 mm column, eluting with iPrOH at a flowrate of 1 mL/min, to yield enantiomer 1 as a white solid, 9 mg, Rt 5.6 min. and enantiomer 2 as a white solid, 9 mg, Rt 9.9 min.

### Example 39

### 3-[5-(3-Chloro-phenyl)-isoxazol-3-yl]-4-[5-(4-difluoromethoxy-phenyl)-4-methyl-4H-[1,2,4]triazol-3-yl]-morpholine

Pyridine (30 μl) and 4-difluoromethoxy-benzoic acid hydrazide (57.9 mg, 0.29 mmol) were added to a solution of 3-[5-(3-chloro-phenyl)-isoxazol-3-yl]-N-methyl-morpholine-4-carboximidothioic acid methyl ester (960 mg, 0.27 mmol) in ethanol. The mixture was stirred at 75° C. for 48 h, and then diluted with dichloromethane (8 mL). The organic phase was sequentially washed with water (4×10 mL) and brine (10 mL), dried (sodium sulfate), filtered and concentrated in vacuo. Chromatography (silica gel, 10% dichloromethane in ethyl acetate) yielded the title compound as clear oil (18 mg). ¹H NMR (CDCl₃), δ (ppm): 7.67 (m, 4H), 7.39 (m, 2H), 7.23 (d, 2H), 6.66 (s, 1H), 6.58 (t, 1H), 4.80 (dd, 1H), 4.25 (dd, 1H), 4.07 (m, 3H), 3.61 (s, 3H), 3.40 (m, 2H).

### Example 40

### 3-[3-(3-Chloro-phenyl)-[1,2,4]oxadiazol-5-yl]-4-(4-methyl-5-pyridin-4-yl-4H-[1,2,4]triazol-3-yl)-morpholine

Pyridine (30 μl) and isonicotinic acid hydrazide (60 mg, 0.29 mmol) were added to 3-[3-(3-Chloro-phenyl)-[1,2,4]oxadiazol-5-yl]-methylmorpholine-4-carboximidothioic acid methyl ester (101 mg, 0.44 mmol) in ethanol, and the mixture was stirred at 75° C. for 48 h, and the mixture was diluted with dichloromethane (8 mL). The organic phase was sequentially washed with water (4×10 mL) and brine (10 mL), dried (sodium sulfate), filtered and concentrated in vacuo. Chromatography (silica gel, 10% dichloromethane in ethyl acetate) yielded the title compound as clear oil (40 mg, 33%). ¹H NMR (CDCl₃), δ (ppm): 8.78 (d, 2H), 8.03 (d, 1H), 7.92 (dd, 1H), 7.63 (d, 2H), 7.46 (dd, 1H), 7.40 (t, 1H), 5.14 (dd, 1H), 4.35 (d, 1H), 4.14 (m, 3H), 3.75 (s, 3H), 3.73 (m,1H), 3.39 (m, 1H).

### Example 41

### 3-[3-(3-Chloro-phenyl)-[1,2,4]oxadiazol-5-yl]-4-[5-(4-difluoromethoxy-phenyl)-4-methyl-4H-[1,2,4]triazol-3-yl]-morpholine

3-[3-(3-Chloro-phenyl)-[1,2,4]oxadiazol-5-yl]-N-methyl-morpholine-4-carboximidothioic acid methyl ester (100 mg, 0.28 mmol), 4-difluoromethoxy-benzoic acid hydrazide (60.2 mg, 0.30 mmol) and pyridine (4 drops) in ethanol (10 mL) were heated at 75° C. for 24 hours. After cooling, the reaction mixture was diluted with ethyl acetate and then washed with water (5 times) and brine, dried over anhydrous sodium sulfate, filtered and concentrated. Chromatography (silica gel, 1-2% methanol in dichloromethane) afforded the title compound (99.5 mg, 73%). ¹H NMR (CDCl₃) δ (ppm): 8.03 (m, 1H), 7.93 (m, 1H), 7.67 (m, 2H), 7.46 (m, 1H), 7.42 (m, 1H), 7.25 (m, 2H), 6.59 (t, 1H), 5.13 (m, 1H), 4.37 (m,1H), 4.16 (m, 1H), 4.01 (m, 2H), 3.66 (m, 1H), 3.67 (s, 3H), 3.36 (m, 1H).

### Example 42

### 3-[3-(3-Chloro-phenyl)-[1,2,4]oxadiazol-5-yl]-4-(4-methyl-5-pyridin-4-yl-4H-[1,2,4]triazol-3-yl)-piperazine-1-carboxylic acid tert-butyl ester

3-[3-(3-Chloro-phenyl)-[1,2,4]oxadiazol-5-yl]-4-(methylimino-methylsulfanyl-methyl)-piperazine-1-carboxylic acid tert-butyl ester (211.6 mg, 0.47 mmol) and isonicotinic hydrazide (96.5 mg, 0.70 mmol) in ethanol (6 mL) were heated at 80° C. for 24 hours. After cooling, the mixture was diluted with ethyl acetate and washed with water (5 times) and brine, dried over anhydrous sodium sulfate, filtered and concentrated. Chromatography (silica gel, 0-5% 2M methanolic ammonia in 1:1 ethyl acetate:dichloromethane) afforded the title compound (168.5 mg, 69%, colorless oil). ¹H NMR (CDCl₃) δ (ppm): 8.77 (m, 2H), 8.04 (s, 1H), 7.94 (m, 1H), 7.62 (m, 2H), 7.44 (m, 2H), 5.08 (m, 1H), 4.15 (m, 1H) 4.06 (m, 1H), 3.75 (m, 3H), 3.73 (s, 3H), 3.32 (m, 1H), 1.43 (bs, 9H).

### Example 43

### 2-[3-(3-Chloro-phenyl)-[1,2,4]oxadiazol-5-yl]-1-(4-methyl-5-pyridin-4-yl-4H-1,2,4]triazol-3-yl)-piperazine

Trifluoroacetic acid (1.5 mL) was added to a solution of 3-[3-(3-chloro-phenyl)-[1,2,4]oxadiazol-5-yl]-4-(4-methyl-5-pyridin-4-yl-4H-[1,2,4]triazol-3-yl)-piperazine-1-carboxylic acid tert-butyl ester (164 mg, 0.31 mmol) in dichloromethane (3 mL) at 0° C. and stirred for 2.5 hours. After the mixture was concentrated, the residue was diluted with dichloromethane and then washed with saturated sodium bicarbonate, dried over anhydrous sodium sulfate, filtered and concentrated to afford the title compound (109 mg, 83%, white foam solid). ¹H NMR (CDCl₃) δ (ppm): 8.75 (m, 2H), 8.02 (m, 1H), 7.93 (m, 1H), 7.62 (m, 2H), 7.43 (m, 2H), 5.01 (m, 1H), 3.73 (s, 3H), 3.62 (m, 2H), 3.40 (m, 1H), 3.22 (m, 3H).

### Example 44

### 2-[3-(3-Chloro-phenyl)-[1,2,4]oxadiazol-5-yl]-4-methyl-1-(4-methyl-5-pyridin-4-yl-4H-[1,2,4]triazol-3-yl)-piperazine

Formic acid (0.1 mL), formaldehyde (37 wt. % solution in water, 0.1 mL) and sodium cyanoborohydride (1.0 M in THF, 0.1 mL) were added to a solution of 2-[3-(3-chloro-phenyl)-[1,2,4]oxadiazol-5-yl]-1-(4-methyl-5-pyridin-4-yl-4H-1,2,4]triazol-3-yl)-piperazine (50.3 mg, 0.12 mmol) in methanol (0.8 mL) at room temperature. After stirring for 30 minutes, the mixture was diluted with water and extracted with chloroform (4 times), dried over anhydrous sodium sulfate, filtered and concentrated. Chromatography (silica gel, 1-5% 2M methanolic ammonia in dichloromethane) afforded the title compound (90%). ¹H NMR (CDCl₃) δ (ppm): 8.77 (m, 2H), 8.03 (m, 1H), 7.93 (m, 1H), 7.63 (m, 2H), 7.42 (m, 2H), 5.21 (m, 1H), 3.74 (s, 3H), 3.70 (m, 1H), 3.43 (m, 1H), 3.09 (m, 2H), 2.70 (m, 2H), 2.41 (s, 3H).

### Example 45

### 3-[3-(3-Chloro-phenyl)-[1,2,4]oxadiazol-5-yl]-4-[5-(4-difluoromethoxy-phenyl)-4-methyl-4H-[1,2,4]triazol-3-yl]-piperazine-1-carboxylic acid tert-butyl ester

3-[3-(3-Chloro-phenyl)-[1,2,4]oxadiazol-5-yl]-4-(methylimino-methylsulfanyl-methyl)-piperazine-1-carboxylic acid tert-butyl ester (211.3 mg, 0.47 mmol), 4-difluoromethoxy-benzoic acid hydrazide (99.2 mg, 0.49 mmol) and pyridine (8 drops) in ethanol were heated at 75° C. for three days. After cooling, the reaction mixture was diluted with ethyl acetate and then washed with water (5 times) and brine, dried over anhydrous sodium sulfate, filtered and concentrated. Chromatography (silica gel, ethyl acetate:hexanes:dichloromethane 3:1:4 to 100% ethyl acetate) afforded the title compound.

### Example 46

### 2-[3-(3-Chloro-phenyl)-[1,2,4]oxadiazol-5-yl]-1-[5-(4-difluoromethoxy-phenyl)-4-methyl-4H-[1,2,4]triazol-3-yl]-piperazine

Trifluoroacetic acid (1.5 mL) was added to a solution of 3-[3-(3-chloro-phenyl)-[1,2,4]oxadiazol-5-yl]-4-[5-(4-difluoromethoxy-phenyl)-4-methyl-4H-[1,2,4]triazol-3-yl]-piperazine-1-carboxylic acid tert-butyl ester at 0° C. and stirred for 2.5 hours. After the mixture was concentrated, the residue was diluted with dichloromethane and then washed with saturated sodium bicarbonate, dried over anhydrous sodium sulfate filtered, and concentrated. Chromatography (silica gel, 3-4% 2M methanolic ammonia in dichloromethane) afforded the titled compound (white solid, 31% yield over 2 steps). ¹H NMR (CDCl₃) δ (ppm): 8.05 (m, 1H), 7.95 (m, 1H), 7.69 (m, 2H), 7.47 (m, 1H), 7.42 (m, 1H), 7.26 (m, 2H), 6.59 (t, 1H), 5.01 (m, 1H), 3.63 (m, 5H), 3.39 (m, 1H), 3.20 (m, 3H).

### Example 47

### 2-[3-(3-Chloro-phenyl)-[1,2,4]oxadiazol-5-yl]-1-[5-(4-difluoromethoxy-phenyl)-4-methyl-4H-[1,2,4]triazol-3-yl]-4-methyl-piperazine

Formic acid (0.1 mL), formaldehyde (37 wt. % in water, 0.1 mL) and sodium cyanoborohydride (1.0M in THF, 0.1 mL) were added to a solution of 2-[3-(3-chloro-phenyl)-[1,2,4]oxadiazol-5-yl]-1-[5-(4-difluoromethoxy-phenyl)-4-methyl-4H-[1,2,4]triazol-3-yl]-piperazine (27.3 mg, 0.056 mmol) in methanol (0.8 mL) at room temperature. After stirring for 30 minutes, the mixture was diluted with water and extracted with chloroform (3 times), dried over anhydrous sodium sulfate, filtered and concentrated. Chromatography (silica gel, 1-3% methanol in dichloromethane) afforded the titled compound (57%). ¹H NMR (CDCl₃) δ (ppm): 8.03 (m, 1H), 7.93 (m, 1H), 7.68 (m, 2H), 7.46 (m, 1H), 7.42 (m, 1H), 7.25 (m, 2H), 6.59 (t, 1H), 5.20 (m, 1H), 3.68 (m,1H), 3.66 (s, 3H), 3.40 (m, 1H), 3.12 (m, 1H), 3.02 (m, 1H), 2.69 (m, 2H), 2.40 (s, 3H).

### Example 48

### 2-[2-(3-Chlorophenyl)-2H-tetrazol-5-yl]-1-{5-[4-(difluoromethoxy)phenyl]-4-methyl-4H-1,2,4-triazol-3-yl}piperidine

2-[2-(3-Chloro-phenyl)-2H-tetrazol-5-yl]-N-methyl-piperidine-1-carboximidothioic acid methyl ester (70 mg, 0.2 mmol) was mixed with 4-difluoromethoxy-benzoic acid hydrazide (40.4 mg, 0.2 mmol) in ethanol at 80° C. overnight. The reaction mixture was diluted with water and extracted with dichloromethane. The dichloromethane layer was dried and purified by chromatography (ethyl acetate) to give the title compound (37 mg, 38%). ¹H NMR (CDCl₃), δ (ppm): 8.09 (s, 1H), 7.99 (m, 1H), 7.66 (d, 2H), 7.46 (m, 2H), 7.24 (d, 2H), 6.58 (t, 1H), 5.10 (m, 1H), 3.66 (s, 3H), 3.48 (m, 1H), 3.30 (m, 1H), 1.70-2.30 (m, 6H).

### Example 49

### 4-(5-{2-[2-(3-chlorophenyl)-2H-tetrazol-5-yl]piperidin-1-yl}-4-methyl-4H-1,2,4-triazol-3-yl)pyridine

2-[2-(3-Chloro-phenyl)-2H-tetrazol-5-yl]-N-methyl-piperidine-1-carboximidothioic acid methyl ester (70 mg, 0.2 mmol) was mixed with isonicotinic acid hydrazide (33.2 mg, 0.2 mmol) in ethanol at 80° C. overnight. The reaction mixture was diluted with water and extracted with dichloromethane. The dichloromethane layer was dried and purified by chromatography (ethyl acetate) to give the title compound (34 mg, 40.3%). ¹H NMR (CDCl₃), δ (ppm): 8.74 (d, 2H), 8.07 (s, 1H), 7.96 (m, 1H), 7.61 (d, 2H), 7.45 (m, 2H), 5.11(m, 1H), 3.73 (s, 3H), 3.48 (m, 1H), 3.30 (m, 1H), 1.70-2.30 (m, 6H).

### Example 50

### 2-[2-(3-Chlorophenyl)-2H-tetrazol-5-yl]-1-[5-(4-methoxyphenyl)-4-methyl-4H-1,2,4-triazol-3-yl]piperidine

2-[2-(3-Chloro-phenyl)-2H-tetrazol-5-yl]-N-methyl-piperidine-1-carboximidothioic acid methyl ester (70 mg, 0.2 mmol) was mixed with 4-methoxy-benzoic acid hydrazide (33.2 mg, 0.2 mmol) in ethanol at 80° C. overnight. The reaction mixture was diluted with water and extracted with dichloromethane. The dichloromethane layer was dried and purified by chromatography (ethyl acetate) to give the title compound (20.2 mg, 22.4%). ¹H NMR (CDCl₃), δ (ppm): 8.09 (s, 1H), 7.98 (m, 1H), 7.57 (d, 2H), 7.45 (m, 2H), 7.99 (d, 2H), 5.10 (m, 1H), 3.86 (s, 3H), 3.63 (s, 3H), 3.48 (m, 1H), 3.29 (m, 1H), 1.70-2.30 (m, 6H).

### Example 51

### [4-(5-{2-[2-(3-chlorophenyl)-2H-tetrazol-5-yl]piperidin-1-yl}-4-methyl-4H-1,2,4-triazol-3-yl)phenyl]dimethylamine

2-[2-(3-Chloro-phenyl)-2H-tetrazol-5-yl]-N-methyl-piperidine-1-carboximidothioic acid methyl ester (70 mg, 0.2 mmol) was mixed with 4-methoxy-benzoic acid hydrazide (27.4 mg, 0.2 mmol) in ethanol at 80° C. overnight. The reaction mixture was diluted with water and extracted with dichloromethane. The dichloromethane layer was dried and purified by chromatography (ethyl acetate) to give the title compound (20.2 mg, 21.6%). ¹H NMR (CDCl₃), δ (ppm): 8.10 (s, 1H), 7.97 (m, 1H), 7.48 (m, 4H), 6.75 (d, 2H), 5.09 (m, 1H), 3.63 (s, 3H), 3.48 (m, 1H), 3.29 (m, 1H), 3.02 (s, 3H), 1.70-2.30 (m, 6H).

Enantiomers were separated using a Chiralpak AD 4.6×250 mm column, eluting with iPrOH at a flowrate of 2 mL/min, to yield enantiomer 1 as a white foam, 2.6 mg, Rt 6.3 min. and enantiomer 2 as a white foam, 2.6 mg, Rt 7.1 min.

### Example 52

### [4-(5-{2-[2-(3-Chloro-phenyl)-2H-tetrazol-5-yl]-piperidin-1-yl}-4-methyl-4H-[1,2,4]triazol-3-yl)-benzyl]-dimethyl-amine

2-[2-(3-Chloro-phenyl)-2H-tetrazol-5-yl]-N-methyl-piperidine-1-carboximidothioic acid methyl ester (49.9 mg, 0.1422 mmol) was mixed with 4-dimethylaminomethyl-benzoic acid hydrazide (30 mg, 0.156 mmol) in ethanol (1.2 mL) at 100° C. overnight. The reaction mixture was diluted with ethyl acetate, washed with water×3, purified by chromatography with (2˜3% 2M methanolic ammonia in chloroform) to give the title compound (9.2 mg, 13.5%) as an off-white solid. ¹H NMR (CDCl₃), δ (ppm): 8.09 (s, 1H), 7.98 (m, 1H), 7.60 (d, 2H), 7.45 (m, 4H), 5.11 (m, 1H), 3.66 (s, 3H), 3.48 (s plus m, 3H), 3.30 (m, 1H), 2.28 (s, 6H), 1.60˜2.20 (m, 6H).

### Example 53

### {2-[4-(5-{2-[2-(3-Chloro-phenyl)-2H-tetrazol-5-yl]-piperidin-1-yl}-4-methyl-4H-[1,2,4]triazol-3-yl)-phenoxy]-ethyl}-dimethyl-amine

2-[2-(3-Chloro-phenyl)-2H-tetrazol-5-yl]-N-methyl-piperidine-1-carboximidothioic acid methyl ester (85 mg, 0.242 mmol) was mixed with 4-(2-dimethylamino-ethoxy)-benzoic acid hydrazide (75.7 mg, 0.339 mmol) in ethanol (1.2 mL) at 100° C. overnight. The reaction mixture was diluted with dichloromethane, washed with water (×3), purified by chromatography (2˜3% 2M methanolic ammonia in chloroform) to give the title compound (32 mg, 26%) as a yellow sticky oil. ¹H NMR (CDCl₃), δ (ppm): 8.09 (s, 1H), 7.97 (m, 1H), 7.56 (d, 2H), 7.44 (m, 2H), 7.01 (d, 2H), 5.09 (m, 1H), 4.11 (t, 2H), 3.62 (s, 3H), 3.65 (m, 1H), 3.44 (m, 1H), 2.76 (t, 2H), 2.36 (s, 6H), 1.60˜2.30 (m, 6H).

### Examples 54a and 54b

### (R)-3-[3-(3-Chloro-phenyl)-[1,2,4]oxadiazol-5-yl]-4-(4-methyl-5-pyridin-4-yl-4H-[1,2,4]triazol-3-yl)-morpholine and

### (S) 3-[3-(3-Chloro-phenyl)-[1,2,4]oxadiazol-5-yl]-4-(4-methyl-5-pyridin-4-yl-4H-[1,2,4]triazol-3-yl)-morpholine

The two enantiomers were isolated from racemic 3-[3-(3-Chloro-phenyl)-[1,2,4]oxadiazol-5-yl]-4-(4-methyl-5-pyridin-4-yl-4H-[1,2,4]triazol-3-yl)-morpholine using chiral HPLC column (Chiralpak AD) with Hexane/Isopropanol (20:80); Enantiomer 1 has retention time of 7.5 minutes whereas Enantiomer 2 has retention time of 8.7 minutes.

### Examples 55a and 55b

### (R)-2-[2-(3-Chlorophenyl)-2H-tetrazol-5-yl]-1-{5-[4-(difluoromethoxy)phenyl]-4-methyl-4H-1,2,4-triazol-3-yl}piperidine

### (S)-2-[2-(3-Chlorophenyl)-2H-tetrazol-5-yl]-{5-[4-(difluoromethoxy)phenyl]-4-methyl-4H-1,2,4-triazol-3-yl}piperidine

2-[2-(3-Chlorophenyl)-2H-tetrazol-5-yl]-1-{5-[4-(difluoromethoxy)phenyl]-4-methyl-4H-1,2,4-triazol-3-yl}piperidine was separated by Chiralpak AD (4.6×250) with ethanol:isoproanol (1:1) at 1.0 mL/min flow rate to give two enatiomers 13.3 mg (Rt=14.2 min) and 11.9 mg (Rt=18.7 min).

### Examples 56a and 56b

### (R)-4-(5-[2-[2-(3-Chlorophenyl)-2H-tetrazol-5-yl]piperidin-1-yl]-4-methyl-4H-1,2,4-triazol-3-yl)pyridine

### (S)-4-(5-{2-[2-(3-Chlorophenyl)-2H-tetrazol-5-yl]piperidin-1-yl}-4-methyl-4H-1,2,4-triazol-3-yl)pyridine

The product was separated by Chiralpak AD (4.6×250) with ethanol:isopropanol (1:1) at 1.0 mL/min flow rate to give two enatiomers 9.5 mg (Rt=11.6 min) and 10.8 mg (Rt=16.8 min).

### Example 57

### 5-Fluoronicotinohydrazide

Hydrazine monohydrate 98% (4.9 mL, 101.1 mmol) was added to a solution of ethyl 5-fluoronicotinate (1.71 g, 10.1 mmol) in EtOH (35 mL) under argon. The reaction was allowed to stir at room temperature for five hours. The reaction was concentrated and triturated with hexane to give the title compound (light yellow solid, 1.462 g, 93%). ¹H NMR CD₃OD δ (ppm): 8.82 (s, 1H), 8.65 (m, 1H), 8.01 (dm, 1H).

### Example 58

### 2-Methylisonicotinohydrazide

HOBt (950 mg, 6.99 mmol), and EDCI (1.34 g, 6.99 mmol) were added to a suspension of 2-chloro-6-methylisonicotinic acid (1 g, 5.83 mmol) in acetonitrile (15 ml) at room temperature. After 1 h a solution of hydrazine monohydrate (0.56 ml, 11.66 mmol) and cyclohexene (0.15 mL, 1.5 mmol) in acetonitrile (5 ml) was added drop-wise at 0° C. The mixture was stirred overnight and allowed to warm to room temperature. The solvent was removed in vacuo and the residue was diluted with ethyl acetate, washed with saturated sodium bicarbonate and brine, dried over sodium sulfate, filtered and concentrated to afford 2-chloro-6-methylisonicotinohydrazide (yellow solid, 1.1 g, used without further purification). A hydrogen filled balloon was attached to a flask containing 2-chloro-6-methylpyridine-4-carboxylic acid (1.12 g, 6.03 mmol), palladium 10 wt. % on activated carbon (0.56 g), triethyl amine (3.4 ml) and ethanol (20 ml) and then stirred overnight at room temperature. The reaction mixture was filtered through celite, washed with methanol and concentrated. The residue was triturated with dichloromethane and then filtered to afford 2-methylisonicotinohydrazide (light yellow solid, crude product used without further purification).

### Example 59

### 2-Methoxyisonicotinohydrazide

HOBt (1.73 g, 12.79 mmol), and EDCI (2.45 g, 12.79 mmol) were added to a suspension of 2-chloro-6-methoxyisonicotinic acid (2 g, 10.66 mmol) in acetonitrile (25 mL) at room temperature. After 1 h a solution of hydrazine monohydrate (1.03 ml, 21.32 mmol) and cyclohexene (0.2 mL, 2.0 mmol) in acetonitrile (5 ml) was added drop-wise at 0° C. The mixture was stirred overnight and allowed to warm to room temperature. The solvent was removed in vacuo and the residue was diluted with ethyl acetate, washed with saturated sodium bicarbonate and brine, dried over sodium sulfate, filtered and concentrated to afford 2-chloro-6-methoxyisonicotinohydrazide (light yellow solid, 2.03 g, 95%). A hydrogen filled balloon was attached to a flask containing 2-chloro-6-methylpyridine-4-carboxylic acid (1.83 g, 9.07 mmol), palladium 10 wt. % on activated carbon (0.91 g), triethyl amine (5.5 ml) and ethanol (30 ml) and then stirred overnight at room temperature. The reaction mixture was filtered through celite, washed with methanol and concentrated. The residue was triturated with dichloromethane and then filtered to afford 2-methoxyisonicotinohydrazide (light yellow solid, crude product used without further purification).

### Example 60

### 3-[2-(3-chlorophenyl)-2H-tetrazol-5-yl]-N-methylmorpholine-4-carbothioamide

To a stirred solution of 3-[2-(3-chlorophenyl)-2H-tetrazol-5-yl]morpholine (550 mg, 2.07 mol) in chloroform (8 mL) was added methyl isothiocyanate (227 mg, 3.1 mmol). The solution was stirred at room temperature overnight, concentrated and triturated with diethyl ether to yield the title compound as (white solid, 608 mg, 86.7%). ¹H NMR (300 MHz, CDCl₃) δ (ppm): 8.13 (s, 1H), 8.03 (dm, 1H), 7.5 (m, 2H), 6.69 (m, 1H), 6.04 (m, 1H), 4.58 (d, 1H), 4.02 (m, 3H), 3.74 (m, 2H), 3.24 (d, 3H).

### Example 61

### Methyl 3-[2-(3-chlorophenyl)-2H-tetrazol-5-yl]-N-methylmorpholine-4-carbimidothioate

To a solution of 3-[2-(3-chlorophenyl)-2H-tetrazol-5-yl]-N-methylmorpholine-4-carbothioamid (608 mg, 1.79 mmol) in methanol (12 mL) was added CH₃I (224 μL, 3.59 mmol). The solution was heated to reflux for 1.5 h, then cooled to room temperature and diluted with dichloromethane and washed with NaHCO₃ (aq). The aqueous phase was re-extracted with dichloromethane and the combined organics were dried (Na₂SO₄), filtered, and concentrated under reduced pressure to yield the title compound in quantitative yield. ¹H NMR (300 MHz, CDCl₃) δ (ppm): 8.15 (s, 1H), 8.04 (dm, 1H), 7.48 (m, 2H), 5.65 (t, 1H), 4.45 (dd, 1H), 4.03 (dd, 1H), 3.93 (dt, 1H), 3.79 (dm, 1H), 3.72 (td, 1H), 3.59 (tm, 1H), 3.25 (s, 3H), 2.38 (s, 3H).

### Example 62

### 3-(N-Hydroxycarbamimidoyl)-morpholine-4-carboxylic acid tert-butyl ester

3-Cyano-morpholine-4-carboxylic acid tert-butyl ester (600 mg, 2.83 mmol) in methanol (20 mL) was added to a solution of hydroxylamine hydrochloride (982 mg, 14.13 mmol) and sodium carbonate (1.498 g, 14.19 mmol) in deionized water (20 mL). The resulting solution was heated to reflux overnight, then cooled to room temperature and the methanol was removed in vacuo. The product was extracted two times with ethyl acetate, then a third time after adding sodium chloride to saturate the aqueous phase. The solvent was removed in vacuo to yield the title compound (sticky off-white solid, 466.8 mg, 67%). ¹H NMR (300 MHz, CDCl₃) δ (ppm): 1.50 (s, 9H); 3.23 (td, J=11 Hz, 3 Hz, 1H); 3.55 (m, 2H); 3.81 (m, 2H); 4.58 (s, broad, 1H); 4.92 (s, broad, 1H).

### Example 63

### 3-[5-(3-Chloro-phenyl)-[1,2,4]oxadiazol-3-yl]-morpholine-4-carboxylic acid tert-butyl ester

To a stirred solution of 3-(N-hydroxycarbamimidoyl)-morpholine-4-carboxylic acid tert-butyl ester (300 mg, 1.22 mmol), 3-chloro-benzoic acid (193.4 mg, 1.24 mmol) and HOBt (181.8 mg, 1.35 mmol) in dimethylformamide (4 mL) was added EDCI (236.8 mg, 1.24 mmol). The solution was stirred overnight at room temperature, then diluted with dichloromethane and washed with water. The aqueous phase was re-extracted with dichloromethane and the combined organics were dried (Na₂SO₄), filtered and concentrated under reduced pressure. The crude intermediate was filtered through silica gel using 10% methanol in dichloromethane to remove trace HOBt. The eluent was concentrated under reduced pressure, then dissolved in dimethylformamide (3 mL) and heated to 130° C. for 90 min. Removal of the solvent in vacuo yielded the title compound (300 mg, 67%). ¹H NMR (300 MHz, CDCl₃) δ (ppm): 1.51 (s, 9H); 3.54 (m, 3H); 3.89 (m, 2H); 4.51 (m, 2H); 7.47 (m, 1H); 7.58 (m, 1H); 8.02 (m, 2H).

### Example 64

### 3-[5-(3-Chloro-phenyl)-[1,2,4]oxadiazol-3-yl]-morpholine

A solution of trifluoroacetic acid (4 mL) in dichloromethane (2 mL) was added to a solution of 3-[5-(3-chloro-phenyl)-[1,2,4]oxadiazol-3-yl]-morpholine-4-carboxylic acid tert-butyl ester (approx. 200 mg) in dichloromethane (2 mL). The resulting solution was stirred at room temperature for 30 min, then diluted with dichloromethane and a small volume of water. The aqueous phase was neutralized with solid sodium bicarbonate, then deionized water was added and the organic phase was separated. The aqueous phase was re-extracted with dichloromethane and the combined organics were dried, filtered and concentrated under reduced pressure to yield the title compound (144.9 mg, quantitative). ¹H NMR (300 MHz, CDCl₃) δ (ppm): 3.10 (m, 2H); 3.72 (m, 1H); 3.85 (m, 2H); 4.18 (dd, J=11 Hz, 3 Hz, 1H); 4.27 (dd, J=8 Hz, 3 Hz, 1H); 7.49 (t, J=8 Hz, 1H); 7.60 (m, 1H); 8.04 (m, 2H).

### Example 65

### 3-[5-(3-Chloro-phenyl)-[1,2,4]oxadiazol-3-yl]-morpholine-4-carbothioicacid methylamide

To a stirred solution of 3-[5-(3-chloro-phenyl)-[1,2,4]oxadiazol-3-yl]-morpholine (184.8 mg, 0.696 mol) in chloroform (5 mL) was added methyl isothiocyanate (54.7 μL, 0.800 mmol). The solution was stirred at room temperature overnight, diluted with dichloromethane and washed with water. The aqueous phase was re-extracted with dichloromethane and the combined organics were dried (Na₂SO₄) filtered and concentrated under reduced pressure. The crude product was chromatographed in 60% ethyl acetate in hexanes to yield the title compound as off-white crystals. ¹H NMR (300 MHz, CDCl₃) δ (ppm): 3.06 (d, J=4.2 Hz, 3H); 3.61 (quintet of d, J=12 Hz, 3 Hz, 2H); 3.92 (m, 3H); 4.51 (d, J=12 Hz, 1H); 6.51 (s, broad, 2H); 7.42 (t, J=7.5 Hz, 1H); 7.51 (m, 1H); 7.92 (m, 1H); 8.01 (m, 1H).

### Example 66

### 3-[5-(3-Chloro-phenyl)-[1,2,4]oxadiazol-3-yl]-N-methyl-morpholine-4-carboximidothioc acid methyl ester

To a solution of 3-[5-(3-chloro-phenyl)-[1,2,4]oxadiazol-3-yl]-morpholine-4-carbothioic acid methylamide (137.6 mg, 0.41 mmol) in methanol (3 mL) was added CH₃I (50.6 μL, 0.82 mmol). The solution was heated to reflux for 1.5 h, then cooled to room temperature and diluted with dichloromethane and washed with NaHCO₃ (aq). The aqueous phase was re-extracted with dichloromethane and the combined organics were dried (Na₂SO₄), filtered, and concentrated under reduced pressure to yield the title compound in quantitative yield. ¹H NMR (300 MHz, CDCl₃) δ (ppm): 2.34 (s, 3H); 3.24 (s, 3H); 3.61 (quintuplet of d, J=12 Hz, 3.3 Hz, 2H); 3.80 (d, J=12 Hz, 1H); 3.91 (m, 2H); 4.40 (dd, J=12 Hz, 2 Hz, 1H); 5.46 (s, broad, 1H); 7.43 (t, J=8.1 Hz, 1H); 7.52 (m, 1H); 7.96 (d, J=7.5 Hz, 1H); 8.07 (m, 1H).

### Example 67

### tert-Butyl 3-[3-(3-iodophenyl)-1,2,4-oxadiazol-5-yl]morpholine-4-carboxylate

Isobutyl chloroformate (1.56 mL, 12.0 mmol) was added to a solution of morpholine-3,4-dicarboxylic acid 4-tert-butyl ester (2.528 g, 10.9 mmol) and triethylamine (3.8 mL, 27.3 mmol) in THF (35 mL) at 0° C. and the mixture was stirred for 2 hours. 3-Iodo-N-hydroxy-benzamidine (2.86 g, 10.9 mmol) was added and the mixture was stirred 1 h at room temperature, and the solvent was removed in vacuo. The acyclic ester intermediate was used without further purification. DMF (25 mL) was added and the mixture was heated at 120° C. overnight. The product was extracted into ethyl acetate and the organic layer was washed with water and brine, dried over anhydrous sodium sulfate, filtered and concentrated in vacuo. Chromatography (silica gel, 10-20% ethyl acetate in hexane) yielded the title compound (light yellow oil, 3.0403 g, 61%) which appeared as a mixture of rotomers in the NMR. ¹H NMR (CDCl₃) (ppm): 8.44 (s, 1H), 8.06 (d, 1H), 7.85 (d, 1H), 7.23 (t, 1H), 5.41 (br s, 0.5H), 5.24 (br s, 0.5H), 4.52 (br s, 1H), 3.9 (m, 3H), 3.6 (t, 1H), 3.49 (m, 1H), 1.53 (s, 4.5H), 1.46 (s, 4.5H).

### Example 68

### 3-[3-(3-Iodophenyl)-1,2,4-oxadiazol-5-yl]morpholine

A solution of trifluoroacetic acid (9.6 mL) in dichloromethane (25 mL) was added to a solution tert-butyl 3-[3-(3-iodophenyl)-1,2,4-oxadiazol-5-yl]morpholine-4-carboxylate (3.04 g, 6.05 mmol) in dichloromethane (30 mL) and the mixture was stirred at room temperature overnight and concentrated to dryness. The residue was dissolved in ethyl acetate and washed with sodium hydroxide (1N aqueous, 15 mL). The organic phase was washed with brine, dried (sodium sulfate), filtered and concentrated in vacuo. Chromatography (silica gel, 5% 2M methanolic ammonia in dichloromethane) yielded the title compound (yellow oil, 2.1599 g, 91%). ¹H NMR (CDCl₃), δ (ppm): 8.48 (s, 1H), 8.08 (d, 1H), 7.86 (d, 1H), 7.24 (t, 1H), 4.34 (m, 1H), 4.2 (d, 1H), 3.86-3.99 (m, 2H), 3.74 (t, 1H), 3.18 (d, 1H), 3.05 (t, 1H).

### Example 69

### 3-[3-(3-Iodophenyl)-1,2,4-oxadiazol-5-yl]-N-methylmorpholine-4-carbothioamide

Methyl isothiocyanate (575 mg, 7.86 mmol) was added to 3-[3-(3-iodophenyl)-1,2,4-oxadiazol-5-yl]morpholine (2.16 g, 6.05 mmol) in CHCl₃ (50 mL) and the resulting mixture was stirred at 60° C. for 7 h and left at room temperature weekend. The mixture was concentrated in vacuo and the isolated residue was triturated with diethyl ether in hexanes to isolate the title compound (yellow oil, 2.6 g, 100%). ¹H NMR (CDCl₃), δ (ppm): 8.38 (s, 1H), 8.02 (d, 1H), 7.83 (d, 1H), 7.2 (t, 1H), 6.86 (m, 1H), 6.18 (m, 1H), 4.56 (d, 1H), 3.99 (m, 2H), 3.78 (t, 2H), 3.63 (m, 1H), 3.03 (d, 3H).

### Example 70

### Methyl 3-[3-(3-iodophenyl)-1,2,4-oxadiazol-5-yl]-N-methylmorpholine-4-carbimidothioate

Iodomethane (0.11 mL, 1.74 mmol) was added to 3-[3-(3-Iodophenyl)-1,2,4-oxadiazol-5-yl]-N-methylmorpholine-4-carbothioamide (465 mg, 1.08 mmol) in methanol (5 mL) and the resulting mixture was stirred at 75° C. for 4 h. The mixture was cooled to room temperature, concentrated in vacuo, diluted with saturated sodium bicarbonate (aqueous), extracted with dichloromethane. The combined organic phase was dried (sodium sulfate), filtered and concentrated in vacuo to yield the title compound as yellow oil (460 mg, 96%). ¹H NMR (CDCl₃), δ (ppm): 7.44 (s, 1H), 8.05 (d, 1H), 7.84 (d, 1H), 7.22 (t, 1H), 5.46 (m, 1H), 4.36 (dm, 1H), 3.96 (m, 2H), 3.65-3.86 (m, 3H), 3.22 (s, 3H), 2.37 (s, 3H).

### Example 71

### 2-[5-(3-Chloro-phenyl)-isoxazol-3-yl]-pyrrolidine-1-carboxylic acid tert-butyl ester

A solution of 2-formyl-pyrrolidine-1-carboxylic acid tert-butyl ester (4.2 g, 21.1 mmol) (Available from PharmaCore, Inc., 4180 Mendenhall Oaks Parkway, Suite 160, High Point, N.C. 27265, USA or synthesized according to procedure by Beak et. al. J. Org. Chem. 1993, 58, 1109) in dry pyridine (8 mL) was added to an ice-cold solution of hydroxylamine hydrochloride (1.90 g, 27.4 mmol) in dry pyridine (25 mL). The solution was stirred at room temperature overnight. Water (500 mL) was added and the solution was extracted with dichloromethane (3×200 mL). The combined organic layers were washed with brine, dried (sodium sulfate), filtered and concentrated in vacuo. The residue was dissolved in dry DMF (50 mL) and heated to 40° C. A solution of N-chlorosuccinimide (3.10 g, 23.2 mmol) in dry DMF (30 mL) was added and the reaction was stirred at 40° C. for 1.5 h and then at room temperature overnight. A further 844 mg (6.3 mmol) of N-chlorosuccinimide was added and the reaction was stirred at 40° C. for 1.5 h. The reaction mixture was allowed to cool and was diluted with diethyl ether (500 mL) and washed with water (3×300 mL) followed by brine (100 mL). The organic layer was dried (sodium sulfate), filtered and concentrated in vacuo. The residue was dissolved in dry dichloromethane (30 mL) and added to an ice-cold solution of 1-Chloro-3-ethynyl-benzene and triethylamine in dry dichloromethane (25 mL). The reaction was stirred overnight at room temperature and the solvent was removed in vacuo. The residue was dissolved in ethyl acetate (300 mL) and washed with water (3×100 mL) and brine (100 mL). The organic layer was dried (sodium sulfate), filtered and concentrated in vacuo. The residue was purified by flash chromatography (heptane:ethyl acetate 8:1-4:1) to give the title compound (3.48 g, 48%) as an oil which solidified on standing. ¹H NMR (400 MHz, CDCl₃, rotamers) δ (ppm): 1.10-1.69 (m, 9H); 1.71-2.44 (m, 4H); 3.34-3.68 (m, 2H); 4.90-5.10 (m, 1H); 6.40-6.55 (m, 1H); 7.37 (bs, 2H); 7.67 (m, 1H); 7.72 (bs, 1H).

### Example 72

### 5-(3-Chloro-phenyl)-3-pyrrolidin-2-yl-isoxazole

2-[5-(3-Chloro-phenyl)-isoxazol-3-yl]-pyrrolidine-1-carboxylic acid tert-butyl ester (3.45 g, 9.9 mmol) was dissolved in dichloromethane (15 mL) and trifluoroacetic acid (15 mL) was added. The solution was stirred at room temperature for 1 h and then concentrated in vacuo. The residue was dissolved in dichloromethane (200 mL) and washed with 1 M aqueous NaOH (200 mL). The aqueous layer was extracted with dichloromethane (2×100 mL) and the combined organic layers were washed with water (100 mL) and brine (100 mL), dried (sodium sulfate), filtered and concentrated in vacuo to give the title compound (2.12 g, 86%). ¹H NMR (400 MHz, CDCl₃) δ (ppm): 1.88 (m, 3H); 2.1 (s, 1H); 3.62 (m, 1H); 3.12 (m, 1H); 4.32 (dd, 1H); 6.51 (s, 1H); 7.35 (m, 1H); 7.61 (m, 1H); 7.72 (s, 1H).

### Example 73

### 2-[5-(3-Chloro-phenyl)-isoxazol-3-yl]-pyrrolidine-1-carbothioic acid methylamide

Methylisothiocyanate (329 mg, 4.5 mmol) was added to 5-(3-chloro-phenyl)-3-pyrrolidin-2-yl-isoxazole (746 mg, 3.0 mmol) in dry dichloromethane (20 mL) at room temperature. The reaction was stirred overnight and concentrated in vacuo. The residue was purified by flash chromatography using a gradient of 20-80% ethyl acetate in heptane to give the title compound (580 mg, 60%). ¹H NMR (400 MHz, CDCl₃) δ (ppm): 2.21 (m, 3H); 2.37 (m, 1H); 3.11 (d, 3H); 3.79 (m, 2H); 5.53 (bs, 1H); 6.57 (s, 1H); 7.39 (m, 1H), 7.68 (m, 1H); 7.73 (m, 1H).

### Example 74

### 2-[5-(3-Chloro-phenyl)-isoxazol-3-yl]-pyrrolidine-1-carbothioic acid cyclopropylamide

Cyclopropylisothiocyanate (446 mg, 4.5 mmol) was added to 5-(3-chloro-phenyl)-3-pyrrolidin-2-yl-isoxazole (746 mg, 3.0 mmol) in dry dichloromethane (20 mL) at room temperature. The reaction was stirred overnight and concentrated in vacuo. The residue was purified by flash chromatography using a gradient of 20-80% ethyl acetate in heptane to give the title compound (585 mg, 56%). ¹H NMR (400 MHz, CDCl₃) δ (ppm): 0.55 (m, 2H); 0.83 (m, 2H); 2.20 (m, 3H); 2.39 (m, 1H); 3.03 (m, 1H); 3.80 (m, 2H); 5.45 (bs, 1H); 5.87 (bs, 1H); 6.65 (bs, 1H); 7.40 (m, 2H); 7.64 (m, 1H); 7.75 (bs, 1H).

### Example 75

### 2-[5-(3-Chloro-phenyl)-isoxazol-3-yl]-N-methyl-pyrrolidine-1-carboximidothioic acid methyl ester

A suspension of 2-[5-(3-chloro-phenyl)-isoxazol-3-yl]-pyrrolidine-1-carbothioic acid methylamide (520 mg, 1.61 mmol) and methyl iodide (344 mg, 2.42 mmol) in dry methanol (10 mL) was heated by single-node microwave irradiation in a sealed vessel at 110° C. for 15 min. After cooling, the resulting solution was diluted with saturated aqueous sodium bicarbonate (50 mL) and extracted with dichloromethane (3×70 mL). The combined organic layers were washed with brine, dried (sodium sulfate), filtered and concentrated in vacuo to give the title compound as an oil (533 mg, 98%). ¹H NMR (400 MHz, CDCl₃) δ (ppm): 2.00 (m, 3H); 2.12 (m, 1H); 2.26 (s, 3H); 3.23 (s, 3H); 3.63 (m, 1H); 3.71 (m, 1H); 5.39 (m, 1H); 6.39 (s, 1H); 7.37 (m, 2H); 7.63 (m, 1H); 7.72 (bs, 1H).

### Example 76

### a) 2-[5-(3-Chloro-phenyl)-isoxazol-3-yl]-N-cyclopropyl-pyrrolidine-1-carboximidothioic acid methyl ester

A suspension of 2-[5-(3-chloro-phenyl)-isoxazol-3-yl]-pyrrolidine-1-carbothioic acid cyclopropylamide (546 mg, 1.57 mmol) and methyl iodide (335 mg, 2.36 mmol) in dry methanol (10 mL) was heated by single-node microwave irradiation in a sealed vessel at 75° C. for 2 h. After cooling, the resulting solution was diluted with saturated aqueous sodium bicarbonate (40 mL) and extracted with dichloromethane (3×50 mL). The combined organic layers were washed with brine, dried (sodium sulfate), filtered and concentrated in vacuo to give the title compound as an oil (563 mg, 99%). ¹H NMR (400 MHz, CDCl₃) 6 (ppm): 0.51 (m, 1H); 0.60 (m, 1H); 0.69 (m, 2H); 1.96 (m, 2H); 2.12 (m, 1H); 2.29 (m, 1H); 2.33 (m, 3H); 3.10 (m, 1H); 3.59 (m, 1H); 3.66 (m, 1H); 5.28 (m, 1H); 6.37 (s, 1H); 7.35 (m, 2H); 7.61 (m, 1H); 7.70 (bs, 1H).

The following compounds were prepared in a similar manner:

b) 4-[5-(5-{2-[5-(3-Chloro-phenyl)-isoxazol-3-yl]-pyrrolidin-1-yl}-4-cyclopropyl-4H-[1,2,4]triazol-3-yl)-pyridin-2-yl]-morpholine; yield 24 mg, 23%, off-white solid; ¹H NMR CDCl₃ (500 MHz): 8.54 (d, 1H), 7.92 (dd, 1H), 7.70 (s, 1H), 7.59 (m, 1H), 7.38 (m, 2H), 6.67 (d, 1H), 6.50 (s, 1H), 5.54 (dd, 1H), 4.06 (ddd, 1H), 3.83 (m, 4H), 3.61 (m, 1H), 3.58 (m, 4H), 3.21 (m, 1H), 2.54 (m, 1H), 2.29 (m, 1H), 2.21 (m, 1H), 2.14 (m, 1H), 1.11 (m, 2H), 0.99 (m, 1H), 0.52 (m, 1H)

c) 4-[5-(5-{2-[5-(3-Chloro-phenyl)-isoxazol-3-yl]-pyrrolidin-1-yl}-4-methyl-4H-[1,2,4]triazol-3-yl)-pyridin-2-yl]-morpholine; yield 41 mg, 34%, light-yellow solid; ¹H NMR CDCl₃ (500 MHz): 8.38 (d, 1H), 7.81 (dd, 1H), 7.72 (s, 1H), 7.61 (m, 1H), 7.37 (m, 2H), 6.70 (d, 1H), 6.59 (s, 1H), 5.41 (t, 1H), 3.87 (m, 1H), 3.83 (m, 4H), 3.59 (m, 4H), 3.52 (m, 4H), 2.55 (m, 1H), 2.30 (m, 1H), 2.19 (m, 2H)

d) 3-(5-{2-[5-(3-Chloro-phenyl)-isoxazol-3-yl]-pyrrolidin-1-yl}-4-methyl-4H-[1,2,4]triazol-3-yl)-pyridine; yield 72 mg, 64%, off-white solid; ¹H NMR CDCl₃ (500 MHz): 8.84 (d, 1H), 8.68 (dd, 1H), 8.01 (dt, 1H), 7.71 (m 1H), 7.61 (m, 1H), 7.42 (dd, 1H), 7.37 (m, 2H), 6.54 (s, 1H), 5.42 (dd, 1H), 3.90 (dt, 1H), 3.57 (s, 3H), 3.54 (ddd, 1H), 2.56 (dddd, 1H), 2.24 (m, 3H)

e) 4-(5-{2-[5-(3-Chloro-phenyl)-isoxazol-3-yl]-pyrrolidin-1-yl}-4-cyclopropyl-4H-[1,2,4]triazol-3-yl)-pyridine; yield 75 mg, 68%, off-white solid; ¹H NMR CDCl₃ (500 MHz): 8.69 (dd, 2H), 7.70 (m, 3H), 7.59 (m, 1H), 7.36 (m, 2H), 6.52 (s, 1H), 5.58 (dd, 1H), 4.11 (dt, 1H), 3.66 (m, 1H), 3.32 (m, 1H), 2.57 (m, 1H), 2.26 (m, 3H), 2.16 (m, 1H), 1.15 (m, 2H), 1.00 (m, 1H), 0.47 (m, 1H)

### Example 77

### 4-(5-{2-[5-(3-chlorophenyl)isoxazol-3-yl]pyrrolidin-1-yl}-4-methyl-4H-1,2,4-triazol-3-yl)pyridine

A suspension of 2-[5-(3-chloro-phenyl)-isoxazol-3-yl]-N-methyl-pyrrolidine-1-carboximidothioic acid methyl ester (61 mg, 0.18 mmol), isonicotinic acid hydrazide (43.2 mg, 0.32 mmol) and pyridine (18 mg, 0.23 mmol) in ethanol (3 mL) was heated by single-node microwave irradiation in a sealed vial at 130° C. After cooling, the solvent was evaporated and the residue was purified by reversed-phase HPLC using a linear gradient of acetonitrile in 0.15% aqueous trifluoroacetic acid. A second purification by reversed-phase HPLC using a linear gradient of acetonitrile in 0.1 M aqueous ammonium acetate to gave the title compound (18.5 mg, 25%) as a white solid after freeze-drying. ¹H NMR CDCl₃ (500 MHz) δ (ppm): 8.71 (d, 2H), 7.69 (s, 1H), 7.58 (m, 3H), 7.36 (m, 2H), 6.53 (s, 1H), 5.42 (dd, 1H), 3.91 (dt, 1H), 3.60 (s, 3H), 3.54 (ddd, 1H), 2.56 (m, 1H), 2.23 (m, 3H)

### Example 78

### 3-(5-{2-[5-(3-chlorophenyl)isoxazol-3-yl]pyrrolidin-1-yl}-4-cyclopropyl-4H-1,2,4-triazol-3-yl)pyridine

A suspension of 2-[5-(3-chloro-phenyl)-isoxazol-3-yl]-N-cyclopropyl-pyrrolidine-1-carboximidothioic acid methyl ester (76 mg, 0.21 mmol), nicotinic acid hydrazide (43.2 mg, 0.32 mmol) and pyridine (18 mg, 0.23 mmol) in 2.5 ml of 2-propanol was heated by single-node microwave irradiation in a sealed vial at 150° C. After cooling, the solvent was evaporated and the residue was purified by reversed-phase HPLC using a linear gradient of acetonitrile in 0.1 M aqueous ammonium acetate to give the title compound (50.3 mg, 55%) as an off-white solid after freeze-drying. ¹H NMR CDCl₃ (500 MHz) δ (ppm): 9.00 (d, 1H), 8.65 (dd, 1H), 8.13 (dt, 1H), 7.71 (m, 1H), 7.60 (m, 1H), 7.38 (m, 3H), 6.53 (s, 1H), 5.58 (dd, 1H), 4.11 (dt, 1H), 3.65 (ddd, 1H), 3.31 (m, 1H), 2.56 (m, 1H), 2.26 (m, 2H), 2.16 (m, 1H), 1.14 (m, 2H), 0.98 (m, 1H), 0.44 (m, 1H).

### Example 79

### a) 3-[2-(3-chlorophenyl)-2H-tetrazol-5-yl]-4-(4-methyl-5-pyridin-4-yl-4H-1,2,4-triazol-3-yl)morpholine

Isonicotinic acid hydrazide (155.5 mg, 1.13 mmol) was added to methyl 3-[2-(3-chlorophenyl)-2H-tetrazol-5-yl]-N-methylmorpholine-4-carbimidothioate (200 mg, 0.567 mmol) in isopropanol (4 mL). The mixture was stirred at 85-95° C. overnight, and then diluted with dichloromethane (8 mL). The organic phase was sequentially washed with water and brine, dried, filtered and concentrated in vacuo. Trituration with diethyl ether yielded the title compound (white solid, 212 mg, 88%). ¹H NMR (CDCl₃), δ (ppm): 8.76 (d, 2H), 8.1 (s, 1H), 7.99 (m, 1H), 7.62 (m, 2H), 7.48 (m, 2H), 5.2 (dd, 1H), 4.32 (dd, 1H), 4.12 (m, 2H), 4.03 (m, 1H), 3.77 (s, 3H), 3.6 (m, 1H), 3.42 (m, 1H).

The following compounds were prepared in a similar manner:

b) 3-[2-(3-chlorophenyl)-2H-tetrazol-5-yl]-4-(4-methyl-5-pyridin-3-yl-4H-1,2,4-triazol-3-yl)morpholine; yield 89.3 mg, 87.4% white solid; ¹H NMR (CDCl₃), δ (ppm): 8.89 (s, 1H), 8.73 (d, 1H), 8.11 (m, 1H), 8.03 (m, 2H), 7.47 (m, 3H), 5.2 (dd, 1H), 4.33 (dd, 1H), 4.12 (m, 2H), 4.04 (m, 1H), 3.74 (s, 3H), 3.59 (m, 1H), 3.46 (m, 1H)

c) 3-[2-(3-chlorophenyl)-2H-tetrazol-5-yl]-4-[5-(3,5-difluorophenyl)-4-methyl-4H-1,2,4-triazol-3-yl]morpholine; yield 78.9 mg, 71.4% white solid; ¹H NMR (CDCl₃), δ (ppm): 8.1 (s, 1H), 7.99 (m, 1H), 7.48 (m, 2H), 7.23 (m, 2H), 6.94 (tm, 1H), 5.18 (dd, 1H), 4.32 (dd, 1H), 4.11 (m, 2H), 4.02 (m, 1H), 3.73 (s, 3H), 3.57 (dm, 1H), 3.41 (m, 1H)

Pharmacology

The pharmacological properties of the compounds of the invention can be analyzed using standard assays for functional activity. Examples of glutamate receptor assays are well known in the art as described in for example Aramori et al., Neuron 8:757 (1992), Tanabe et al., Neuron 8:169 (1992), Miller et al., J. Neuroscience 15: 6103 (1995), Balazs, et al., J. Neurochemistry 69:151 (1997). The methodology described in these publications is incorporated herein by reference. Conveniently, the compounds of the invention can be studied by means of an assay that measures the mobilization of intracellular calcium, [Ca²⁺]ᵢ in cells expressing mGluR5.

For FLIPR analysis, cells expressing human mGluR5d as described in WO97/05252 were seeded on collagen coated clear bottom 96-well plates with black sides and analysis of [Ca²⁺]ᵢ mobilization was done 24 h after seeding.

FLIPR experiments were done using a laser setting of 0.800 W and a 0.4 second CCD camera shutter speed. Each FLIPR experiment was initiated with 160 μl of buffer present in each well of the cell plate. After each addition of the compound, the fluorescence signal was sampled 50 times at 1 second intervals followed by 3 samples at 5 second intervals. Responses were measured as the peak height of the response within the sample period. EC₅₀ and IC₅₀ determinations were made from data obtained from 8-point concentration response curves (CRC) performed in duplicate. Agonist CRC were generated by scaling all responses to the maximal response observed for the plate. Antagonist block of the agonist challenge was normalized to the average response of the agonist challenge in 14 control wells on the same plate.

We have validated a secondary functional assay for mGluR5d as described in WO97/05252 based on Inositol Phosphate (IP₃) turnover. IP₃ accumulation is measured as an index of receptor mediated phospholipase C turnover. GHEK cells stably expressing the human mGluR5d receptors were incubated with [3H] myo-inositol overnight, washed three times in HEPES buffered saline and pre-incubated for 10 min with 10 mM LiCl. Compounds (agonists) were added and incubated for 30 min at 37° C. Antagonist activity was determined by pre-incubating test compounds for 15 min, then incubating in the presence of glutamate (80 μM) or DHPG (30 μM) for 30 min. Reactions were terminated by the addition of perchloric acid (5%). Samples were collected and neutralized, and inositol phosphates were separated using Gravity-Fed Ion-Exchange Columns.

A detailed protocol for testing the compounds of the invention is provided in the assay below.

Assay of Group I Receptor Antagonist Activity

For FLIPR analysis, cells expressing human mGluR5d as described in WO97/05252 were seeded on collagen coated clear bottom 96-well plates with black sides and analysis of [Ca²⁺]ᵢ mobilization was performed 24 h following seeding. Cell cultures in the 96-well plates were loaded with a 4 μM solution of acetoxymethyl ester form of the fluorescent calcium indicator fluo-3 (Molecular Probes, Eugene, Oreg.) in 0.01% pluronic. All assays were performed in a buffer containing 127 mM NaCl, 5 mM KCl, 2 mM MgCl₂, 0.7 mM NaH₂PO₄, 2 mM CaCl₂, 0.422 mg/ml NaHCO₃, 2.4 mg/ml HEPES, 1.8 mg/ml glucose and 1 mg/ml BSA Fraction IV (pH 7.4).

FLIPR experiments were done using a laser setting of 0.800 W and a 0.4 second CCD camera shutter speed with excitation and emission wavelengths of 488 nm and 562 nm, respectively. Each FLIPR experiment was initiated with 160 μl of buffer present in each well of the cell plate. A 40 μl addition from the antagonist plate was followed by a 50 μL addition from the agonist plate. After each addition the fluorescence signal was sampled 50 times at 1 second intervals followed by 3 samples at 5 second intervals. Responses were measured as the peak height of the response within the sample period.

EC₅₀/IC₅₀ determinations were made from data obtained from 8 points concentration response curves (CRC) performed in duplicate. Agonist CRC were generated by scaling all responses to the maximal response observed for the plate. Antagonist block of the agonist challenge was normalized to the average response of the agonist challenge in 14 control wells on the same plate.

Measurement of Inositol Phosphate Turnover in Intact Whole Cells

GHEK stably expressing the human mGluR5d receptor were seeded onto 24 well poly-L-lysine coated plates at 40×10⁴ cells/well in media containing 1 μCi/well [3H] myo-inositol. Cells were incubated overnight (16 h), then washed three times and incubated for 1 h at 37° C. in HEPES buffered saline (146 mM NaCl, 4.2 mM KCl, 0.5 mM MgCl₂, 0.1% glucose, 20 mM HEPES, pH 7.4) supplemented with 1 unit/ml glutamate pyruvate transaminase and 2 mM pyruvate. Cells were washed once in HEPES buffered saline and pre-incubated for 10 min in HEPES buffered saline containing 10 mM LiCl. Compounds (agonists) were added and incubated at 37° C. for 30 min. Antagonist activity was determined by pre-incubating test compounds for 15 min, then incubating in the presence of glutamate (80 μM) or DHPG (30 μM) for 30 min. The reaction was terminated by the addition of 0.5 ml perchloric acid (5%) on ice, with incubation at 4° C. for at least 30 min. Samples were collected in 15 ml Falcon tubes and inositol phosphates were separated using Dowex columns, as described below.

Assay for Inositol Phosphates Using Gravity-Fed Ion-Exchange Columns

Preparation of Ion-Exchange Columns

Ion-exchange resin (Dowex AG1-X8 formate form, 200-400 mesh, BIORAD) was washed three times with distilled water and stored at 4° C. 1.6 ml resin was added to each column, and washed with 3 ml 2.5 mM HEPES, 0.5 mM EDTA, pH 7.4.

a) Sample Treatment

Samples were collected in 15 ml Falcon tubes and neutralized with 0.375 M HEPES, 0.75 M KOH. 4 ml of HEPES/EDTA (2.5/0.5 mM, pH 7.4) were added to precipitate the potassium perchlorate. Supernatant was added to the prepared Dowex columns.

b) Inositol Phosphate Separation

Elute glycero phosphatidyl inositols with 8 ml 30 mM ammonium formate. Elute total inositol phosphates with 8 ml 700 mM ammonium formate/100 mM formic acid and collect eluate in scintillation vials. Count eluate mixed with 8 ml scintillant.

One aspect of the invention relates to a method for inhibiting activation of mGluR5, comprising treating a cell containing said receptor with an effective amount of the compound of formula I.

Screening for Compounds Active Against TLESR

Adult Labrador retrievers of both genders, trained to stand in a Pavlov sling, are used. Mucosa-to-skin esophagostomies are formed and the dogs are allowed to recover completely before any experiments are done.

Motility Measurement

In brief, after fasting for approximately 17 h with free supply of water, a multilumen sleeve/sidehole assembly (Dentsleeve, Adelaide, South Australia) is introduced through the esophagostomy to measure gastric, lower esophageal sphincter (LES) and esophageal pressures. The assembly is perfused with water using a low-compliance manometric perfusion pump (Dentsleeve, Adelaide, South Australia). An air-perfused tube is passed in the oral direction to measure swallows, and an antimony electrode monitored pH, 3 cm above the LES. All signals are amplified and acquired on a personal computer at 10 Hz.

When a baseline measurement free from fasting gastric/LES phase III motor activity has been obtained, placebo (0.9% NaCl) or test compound is administered intravenously (i.v., 0.5 ml/kg) in a foreleg vein. Ten min after i.v. administration, a nutrient meal (10% peptone, 5% D-glucose, 5% Intralipid, pH 3.0) is infused into the stomach through the central lumen of the assembly at 100 ml/min to a final volume of 30 ml/kg. The infusion of the nutrient meal is followed by air infusion at a rate of 500 ml/min until an intragastric pressure of 10±1 mmHg is obtained. The pressure is then maintained at this level throughout the experiment using the infusion pump for further air infusion or for venting air from the stomach. The experimental time from start of nutrient infusion to end of air insufflation is 45 min. The procedure has been validated as a reliable means of triggering TLESRs.

TLESRs is defined as a decrease in lower esophageal sphincter pressure (with reference to intragastric pressure) at a rate of >1 mmHg/s. The relaxation should not be preceded by a pharyngeal signal <2 s before its onset in which case the relaxation is classified as swallow-induced. The pressure difference between the LES and the stomach should be less than 2 mmHg, and the duration of the complete relaxation longer than 1 s.

Abbreviations

BSA Bovine Serum Albumin

CCD Charge Coupled Device

CRC Concentration Response Curve

DHPG 3,5-dihydroxyphenylglycine;

EDTA Ethylene Diamine Tetraacetic Acid

FLIPR Fluorometric Imaging Plate reader

GHEK GLAST-containing Human Embrionic Kidney

GLAST glutamate/aspartate transporter

HEPES 4-(2-hydroxyethyl)-1-piperazineethanesulfonic acid (buffer)

IP₃ inositol triphosphate

Results

Typical IC₅₀ values as measured in the assays described above are 10 μM or less. In one aspect of the invention the IC₅₀ is below 2 μM. In another aspect of the invention the IC₅₀ is below 0.2 μM. In a further aspect of the invention the IC₅₀ is below 0.05 μM.

| | FLIPR |
|---|---|
| Compound | IC₅₀ |
| 3-[3-(3-chlorophenyl)-1,2,4-oxadiazole-5-yl]4-{5-[4- | 199 nM |
| (difluoromethoxy)phenyl]-4-methyl-4H-1,2,4-triazole-3- | |
| yl}morpholine | |
| 4-(5-{2-[2-(3-chlorophenyl)-2H-tetrazole-5-yl]piperidin- | 40 nM |
| 1-yl}-4-methyl-4H-1,2,4-triazole-3-yl)pyridine | |

1. A compound according to formula I 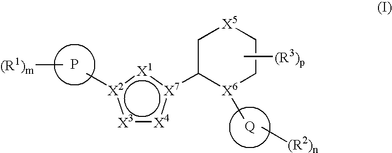
wherein
P is selected from aryl and heteroaryl;
R¹ is attached to P via a carbon atom on ring P and is selected from the group consisting of hydroxy, halo, nitro, C₁₋₆alkylhalo, OC₁₋₆alkylhalo, C₁₋₆alkyl, OC₁₋₆alkyl, C₂₋₆alkenyl, OC₂₋₆alkenyl, C₂₋₆alkynyl, OC₂₋₆alkynyl, C₀₋₆alkylC₃₋₆cycloalkyl, OC₀₋₆alkylC₃₋₆cycloalkyl, C₀₋₆alkylaryl, OC₀₋₆alkylaryl, CHO, (CO)R⁵, O(CO)R⁵, O(CO)OR⁵, O(CNR⁵)OR⁵, C₁₋₆alkylOR⁵, OC₂₋₆alkylOR⁵, C₁₋₆alkyl(CO)R⁵, OC₁₋₆alkyl(CO)R⁵, C₀₋₆alkylCO₂R⁵, OC₁₋₆alkylCO₂R⁵, C₀₋₆alkylcyano, OC₂₋₆alkylcyano, C₀₋₆alkylNR⁵R⁶, OC₂₋₆alkylNR⁵R⁶, C₁₋₆alkyl(CO)NR⁵R⁶, OC₁₋₆alkyl(CO)NR⁵R⁶, C₀₋₆alkylNR⁵(CO)R⁶, OC₂₋₆alkylNR⁵(CO)R⁶, C₀₋₆alkylNR⁵(CO)NR⁵R⁶, C₀₋₆alkylSR⁵, OC₂₋₆alkylSR⁵, C₀₋₆alkyl(SO)R⁵, OC₂₋₆alkyl(SO)R⁵, C₀₋₆alkylSO₂R⁵, OC₂₋₆alkylSO₂R⁵, C₀₋₆alkyl(SO₂)NR⁵R⁶, OC₂₋₆alkyl(SO₂)NR⁵R⁶, C₀₋₆alkylNR⁵(SO₂)R⁶, OC₂₋₆alkylNR⁵(SO₂)R⁶, C₀₋₆alkylNR⁵(SO₂)NR⁵R⁶, OC₂₋₆alkylNR⁵(SO₂)NR¹R⁶, (CO)NR⁵R⁶, O(CO)NR⁵R⁶, NR⁵OR⁶, C₀₋₆alkylNR⁵(CO)OR⁶, OC₂₋₆alkylNR⁵(CO)OR⁶, SO₃R⁵ and a 5- or 6-membered ring containing atoms independently selected from the group consisting of C, N, O and S;
X¹ is selected from the group consisting of: N, NR⁴ and CR⁴;
X² is selected from the group consisting of: C and N;
X³ is selected from the group consisting of: CR⁴, N and O;
X⁴ is selected from the group consisting of: CR⁴, N, NR⁴ and O;
X⁵ is selected from the group consisting of: a bond, CR⁴R^{4′}, NR⁴, O, S, SO and SO₂;
X⁶ is selected from the group consisting of: CR⁴ and N;
X⁷ is selected from the group consisting of: C and N;
R⁴ is independently selected from a group consisting of hydrogen, hydroxy, C₁₋₆alkyl, C₀₋₆alkylcyano, oxo, ═NR⁵, ═NOR⁵, C₁₋₄alkylhalo, halo, C₃₋₇cycloalkyl, O(CO)C₁₋₄alkyl, C₁₋₄alkyl(SO)C₀₋₄alkyl, C₁₋₄alkyl(SO₂)C₀₋₄alkyl, (SO)C₀₋₄alkyl, (SO₂)C₀₋₄alkyl, OC₁₋₄alkyl, C₁₋₄alkylOR⁵ and C₀₋₄alkylNR⁵R⁶;
Q is selected the group consisting of heterocycloalkyl and heteroaryl;
R² and R³ are independently selected from the group consisting of: hydroxy, C₀₋₆alkylcyano, oxo, ═NR⁵, ═NOR⁵, C₁₋₄alkylhalo, halo, C₁₋₆alkyl, C₃₋₆cycloalkyl, C₀₋₆alkylaryl, C₀₋₆alkylheteroaryl, C₁₋₆alkylcycloalkyl, C₀₋₆alkylheterocycloalkyl, OC₁₋₄alkyl, OC₀₋₆alkylaryl, O(CO)C₁₋₄alkyl, (CO)OC₁₋₄alkyl, C₀₋₄alkyl(S)C₀₋₄alkyl, C₁₋₄alkyl(SO)C₀₋₄alkyl, C₁₋₄alkyl(SO₂)C₀₋₄alkyl, (SO)C₀₋₄alkyl, (SO₂)C₀₋₄alkyl, C₁₋₄alkylOR⁵, C₀₋₄alkylNR⁵R⁶ and a 5- or 6-membered ring containing atoms independently selected from C, N, O and S, which ring may optionally be fused with a 5- or 6-membered ring containing atoms independently selected from the group consisting of C, N and O and wherein said ring and said fused ring may be substituted by one or more A;
wherein any C₁₋₆alkyl, aryl, or heteroaryl defined under R¹, R² and R³ may be substituted by one or more A;
A is selected from the group consisting of: hydrogen, hydroxy, halo, nitro, oxo, C₀₋₆alkylcyano, C₀₋₄alkylC₃₋₆cycloalkyl, C₁₋₆alkyl, —OC₁₋₆alkyl, C₁₋₆alkylhalo, OC₁₋₆alkylhalo, C₂₋₆alkenyl, C₀₋₃alkylaryl, C₀₋₆alkylOR⁵, OC₂₋₆alkylOR⁵, C₀₋₆alkylSR⁵, OC₂₋₆alkylSR⁵, (CO)R⁵, O(CO)R⁵, OC₂₋₆alkylcyano, OC₁₋₆alkylCO₂R⁵, O(CO)OR⁵, OC₁₋₆alkyl(CO)R⁵, C₁₋₆alkyl(CO)R⁵, NR⁵OR⁶, C₀₋₆NR⁵R⁶, OC₂₋₆alkylNR⁵R⁶, C₀₋₆alkyl(CO)NR⁵R⁶, OC₁₋₆alkyl(CO)NR⁵R⁶, OC₂₋₆alkylNR⁵(CO)R⁶, C₀₋₆alkylNR⁵(CO)R⁶, C₀₋₆alkylNR⁵(CO)NR⁵R⁶, O(CO)NR⁵R⁶, C₀₋₆alkyl(SO₂)NR⁵R⁶, OC₂₋₆alkyl(SO₂)NR⁵R⁶, C₀₋₆alkylNR⁵(SO₂)R⁶, OC₂₋₆alkylNR⁵(SO₂)R⁶, SO₃R⁵, C₁₋₆alkylNR⁵(SO₂)NR⁵R⁶, OC₂₋₆alkyl(SO₂)R⁵, C₀₋₆alkyl(SO₂)R⁵, C₀₋₆alkyl(SO)R⁵, OC₂₋₆alkyl(SO)R⁵ and a 5- or 6-membered ring containing atoms independently selected from the group consisting of C, N, O and S;
R⁵ and R⁶ are independently selected from, H, C₁₋₆alkyl, C₃₋₇cycloalkyl and aryl;
m is selected from 0, 1, 2, 3 or 4;
n is selected from 0, 1, 2, 3 or 4;
p is selected from 0, 1, 2, 3 or 4; and
a salt or hydrate thereof,
with the proviso that the compound is not:
4,4′-(1,2-piperazinediyl)di-antipyrine;
4,4′-(1,2-piperazinediyl)di-antipyrine dihydrochloride; or
4,4′-(1,2-piperazinediyl)di-antipyrine dipicrate;

2. A compound according to claim 1 wherein m is selected from 1, 2, 3 or 4

3. A compound according to claim 1 wherein X⁷ is C.

4. A compound according to claim 1 wherein X⁵ is selected from the group consisting of CR⁴R^{4′}, NR⁴, O, S, SO and SO₂.

5. A compound according to claim 1 wherein X³ is selected from the group consisting of N and O.

6. A compound according to claim 1 wherein P is aryl.

7. A compound according to claim 6 wherein P is phenyl.

8. A compound according to claim 7 wherein m is selected from the group consisting of 1 and 2.

9. A compound according to claim 1 wherein R¹ is selected from the group consisting of: halo, C₁₋₆alkylhalo, OC₁₋₆alkylhalo, C₁₋₆alkyl, OC₁₋₆alkyl, C₁₋₆alkylOR⁵, C₀₋₆alkylcyano, C₀₋₆alkylNR⁵R⁶.

10. A compound according to claim 9 wherein R¹ is selected from the group consisting of: Cl, F, Me, OMe, CF₃, OCF₃, and CN.

11. A compound according to claim 1 wherein X² is C.

12. A compound according to claim 11 wherein X¹ is N or CR⁴.

13. A compound according to claim 12 wherein when X³ is O, X⁴ is N and when X³ is N, X⁴ is O.

14. A compound according to claim 1 wherein X² is N.

15. A compound according to claim 14 wherein X¹ is N.

16. A compound according to claim 15 wherein X³ is N and X⁴ is N or CR⁴.

17. A compound according to claim 1 wherein X⁶ is N.

18. A compound according to claim 12 wherein X⁵ is selected from the group consisting of a bond, CR⁴R^{4′}, NR⁴ and O.

19. A compound according to claim 13 wherein X⁵ is selected from the group consisting of a bond, O and NR⁴.

20. A compound according to claim 16 wherein X⁵ is selected from the group consisting of O and CR⁴.

21. A compound according to claim 1 wherein R⁴ is selected from the group consisting of: hydrogen, C₁₋₆alkyl, C₁₋₆alkylhalo and halo.

22. A compound according to claim 1 wherein Q is heteroaryl.

23. A compound according to claim 1 wherein Q is selected from the group consisting of:

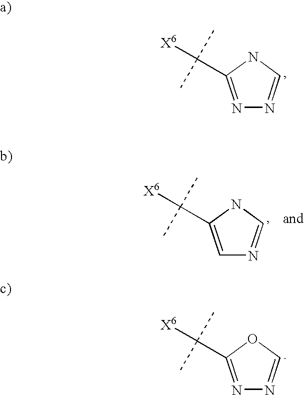

24. A compound according to claim 23 wherein Q is

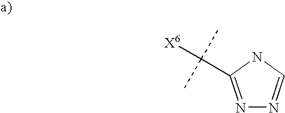

25. A compound according to claim 1 wherein R² and R³ are independently selected from the group consisting of: C₁₋₄alkylhalo, C₁₋₆alkyl, C₃₋₆cycloalkyl, C₀₋₆alkylaryl and C₀₋₆alkylheteroaryl.

26. A compound according to claim 1 wherein A is selected from the group consisting of: hydrogen, hydroxyl, halo, C₀₋₆alkylcyano, C₁₋₆alkyl, —OC₁₋₆alkyl, C₁₋₆alkylhalo, OC₁₋₆alkylhalo.

27. A compound according to claim 1 selected from:
4-(5-{2-[5-(3-Chloro-phenyl)-isoxazol-3-yl]-piperidin-1-yl}-4-methyl-4H [1,2,4]triazol-3-yl)-pyridine
3-[5-(3-Chloro-phenyl)-isoxazol-3-yl]-4-(4-methyl-5-pyridin-4-yl-4H-[1,2,4]triazol-3-yl)-morpholine
3-[5-(3-Chloro-phenyl)-isoxazol-3-yl]-4-[5-(4-difluoromethoxy-phenyl)-4-methyl-4H-[1,2,4]triazol-3-yl]-morpholine
3-[3-(3-Chloro-phenyl)-[1,2,4]oxadiazol-5-yl]-4-(4-methyl-5-pyridin-4-yl-4H-[1,2,4]triazol-3-yl)-morpholine
3-[3-(3-Chloro-phenyl)-[1,2,4]oxadiazol-5-yl]-4-[5-(4-difluoromethoxy-phenyl)-4-methyl-4H-[1,2,4]triazol-3-yl]-morpholine
3-[3-(3-Chloro-phenyl)-[1,2,4]oxadiazol-5-yl]-4-(4-methyl-5-pyridin-4-yl-4H-[1,2,4]triazol-3-yl)-piperazine-1-carboxylic acid tert-butyl ester
2-[3-(3-Chloro-phenyl)-[1,2,4]oxadiazol-5-yl]-1-(4-methyl-5-pyridin-4-yl-4H-1,2,4]triazol-3-yl)-piperazine
2-[3-(3-Chloro-phenyl)-[1,2,4]oxadiazol-5-yl]-4-methyl-1-(4-methyl-5-pyridin-4-yl-4H-[1,2,4]triazol-3-yl)-piperazine
3-[3-(3-Chloro-phenyl)-[1,2,4]oxadiazol-5-yl]-4-[5-(4-difluoromethoxy-phenyl)-4-methyl-4H-[1,2,4]triazol-3-yl]-piperazine-1-carboxylic acid tert-butyl ester
2-[3-(3-Chloro-phenyl)-[1,2,4]oxadiazol-5-yl]-1-[5-(4-difluoromethoxy-phenyl)-4-methyl-4H-[1,2,4]triazol-3-yl]-piperazine
2-[3-(3-Chloro-phenyl)-[1,2,4]oxadiazol-5-yl]-1-[5-(4-difluoromethoxy-phenyl)-4-methyl-4H-[1,2,4]triazol-3-yl]-4-methyl-piperazine
2-[2-(3-Chlorophenyl)-2H-tetrazol-5-yl]-1-{5-[4-(difluoromethoxy)phenyl]-4-methyl-4H-1,2,4-triazol-3-yl}piperidine
4-(5-{2-[2-(3-chlorophenyl)-2H-tetrazol-5-yl]piperidin-1-yl}-4-methyl-4H-1,2,4-triazol-3-yl)pyridine
2-[2-(3-Chlorophenyl)-2H-tetrazol-5-yl]-1-[5-(4-methoxyphenyl)-4-methyl-4H-1,2,4-triazol-3-yl]piperidine
[4-(5-{2-[2-(3-chlorophenyl)-2H-tetrazol-5-yl]piperidin-1-yl}-4-methyl-4H-1,2,4-triazol-3-yl)phenyl]dimethylamine
[4-(5-{2-[2-(3-Chloro-phenyl)-2H-tetrazol-5-yl]-piperidin-1-yl}-4-methyl-4H-[1,2,4]triazol-3-yl)-benzyl]-dimethyl-amine
{2-[4-(5-{2-[2-(3-Chloro-phenyl)-2H-tetrazol-5-yl]-piperidin-1-yl}-4-methyl-4H-[1,2,4]triazol-3-yl)-phenoxy]-ethyl}-dimethyl-amine
(R)-3-[3-(3-Chloro-phenyl)-[1,2,4]oxadiazol-5-yl]-4-(4-methyl-5-pyridin-4-yl-4H-[1,2,4]triazol-3-yl)-morpholine
(S) 3-[3-(3-Chloro-phenyl)-[1,2,4]oxadiazol-5-yl]-4-(4-methyl-5-pyridin-4-yl-4H-[1,2,4]triazol-3-yl)-morpholine
(R)-2-[2-(3-Chlorophenyl)-2H-tetrazol-5-yl]-1-{5-[4-(difluoromethoxy)phenyl]-4-methyl-4H-1,2,4-triazol-3-yl}piperidine
(S)-2-[2-(3-Chlorophenyl)-2H-tetrazol-5-yl]-1-{5-[4-(difluoromethoxy)phenyl]-4-methyl-4H-1,2,4-triazol-3-yl}piperidine
(R)-4-(5-{2-[2-(3-Chlorophenyl)-2H-tetrazol-5-yl]piperidin-1-yl}-4-methyl-4H-1,2,4-triazol-3-yl)pyridine
(S)-4-(5-{2-[2-(3-Chlorophenyl)-2H-tetrazol-5-yl]piperidin-1-yl}-4-methyl-4H-1,2,4-triazol-3-yl)pyridine
4-[5-(5-{2-[5-(3-Chloro-phenyl)-isoxazol-3-yl]-pyrrolidin-1-yl}-4-cyclopropyl-4H-[1,2,4]triazol-3-yl)-pyridin-2-yl]-morpholine,
4-[5-(5-{2-[5-(3-Chloro-phenyl)-isoxazol-3-yl]-pyrrolidin-1-yl}-4-methyl-4H-[1,2,4]triazol-3-yl)-pyridin-2-yl]-morpholine,
3-(5-{2-[5-(3-Chloro-phenyl)-isoxazol-3-yl]-pyrrolidin-1-yl}-4-methyl-4H-[1,2,4]triazol-3-yl)-pyridine,
4-(5-{2-[5-(3-Chloro-phenyl)-isoxazol-3-yl]-pyrrolidin-1-yl}-4-cyclopropyl-4H-[1,2,4]triazol-3-yl)-pyridine,
3-[5-(3-Chloro-phenyl)-[1,2,4]oxadioazol-3-yl]-4-(5-pyridin-4-yl-4H-[1,2,4]triazol-3-yl)-morpholine,
3-[5-(3-chlorophenyl)isoxazol-3-yl]-4-(4-cyclopropyl-5-pyridin-3-yl-4H-1,2,4-triazol-3-yl)morpholine,
3-[5-(3-chlorophenyl)isoxazol-3-yl]-4-(4-cyclopropyl-5-pyridin-4-yl-4H-1,2,4-triazol-3-yl)morpholine,
3-[5-(3-chlorophenyl)isoxazol-3-yl]-4-(4-methyl-5-pyridin-3-yl-4H-1,2,4-triazol-3-yl)morpholine,
3-[5-(3-Chloro-phenyl)-isoxazol-3-yl]-4-[5-(6-methoxy-pyridin-3-yl)-4-methyl-4H-[1,2,4]triazol-3-yl]-morpholine,
3-[3-(3-chlorophenyl)-1,2,4-oxadiazol-5-yl]-4-[5-(2-methoxypyridin-4-yl)-4-methyl-4H-1,2,4-triazol-3-yl]morpholine,
3-[3-(3-chlorophenyl)-1,2,4-oxadiazol-5-yl]-4-[5-(2-methylpyridin-4-yl)-4-methyl-4H-1,2,4-triazol-3-yl]morpholine,
3-[3-(3-chlorophenyl)-1,2,4-oxadiazol-5-yl]-4-[5-(5-fluoropyridin-3-yl)-4-methyl-4H-1,2,4-triazol-3-yl]morpholine,
3-[5-(3-chlorophenyl)isoxazol-3-yl]-4-[5-(5-fluoropyridin-3-yl)-4-methyl-4H-1,2,4-triazol-3-yl]morpholine,
3-[3-(3-chlorophenyl)-1,2,4-oxadiazol-5-yl]-4-(4-methyl-5-pyridin-2-yl-4H-1,2,4-triazol-3-yl)morpholine,
4-[5-(5-fluoropyridin-3-yl)-4-methyl-4H-1,2,4-triazol-3-yl]-3-[3-(3-iodophenyl)-1,2,4-oxadiazol-5-yl]morpholine,
3-[3-(3-iodophenyl)-1,2,4-oxadiazol-5-yl]-4-(4-methyl-5-pyridin-4-yl-4H-1,2,4-triazol-3-yl)morpholine,
3-[5-(3-chlorophenyl)isoxazol-3-yl]-4-[5-(2-methylpyridin-4-yl)-4-methyl-4H-1,2,4-triazol-3-yl]morpholine,
3-[2-(3-chlorophenyl)-2H-tetrazol-5-yl]-4-(4-methyl-5-pyridin-3-yl-4H-1,2,4-triazol-3-yl)morpholine,
3-[2-(3-chlorophenyl)-2H-tetrazol-5-yl]-4-[5-(3,5-difluorophenyl)-4-methyl-4H-1,2,4-triazol-3-yl]morpholine,
3-(5-{2-[5-(3-chlorophenyl)isoxazol-3-yl]pyrrolidin-1-yl}-4-cyclopropyl-4H-1,2,4-triazol-3-yl)pyridine, and
4-(5-{2-[5-(3-chlorophenyl)isoxazol-3-yl]pyrrolidin-1-yl}-4-methyl-4H-1,2,4-triazol-3-yl)pyridine.

28. A pharmaceutical composition comprising as active ingredient a therapeutically effective amount of the compound according to any one of claims 1 to 26, in association with one or more pharmaceutically acceptable diluent, excipients and/or inert carrier.

29. (canceled)

30. The compound according to claim 1, for use in therapy.

31. The compound according to claim 1, for use in treatment of mGluR 5 mediated disorders.

32. Use of the compound according to claim 1, in the manufacture of a medicament for the treatment of mGluR 5 mediated disorders.

33. A method of treatment of mGluR 5 mediated disorders, comprising administering to a mammal, including man in need of such treatment, a therapeutically effective amount of the compound according to claim 1.

34. The method according to claim 33, for use in treatment of neurological disorders.

35. The method according to claim 33, for use in treatment of psychiatric disorders.

36. The method according to claim 33, for use in treatment of chronic and acute pain disorders.

37. The method according to claim 33, for use in treatment of gastrointestinal disorders.

38. A method for inhibiting activation of mGluR 5 receptors, comprising treating a cell containing said receptor with an effective amount of the compound according to claim 1.
